(19) United States
(12) Patent Application Publication
(10) Pub. No.: US 2023/0062248 A1
(43) Pub. Date: Mar. 2, 2023

(54) METHODS FOR PREDICTING IXABEPILONE RESPONSIVENESS IN CANCER PATIENTS

(71) Applicant: Allarity Therapeutics Europe ApS, Hørsholm (DK)

(72) Inventor: Steen KNUDSEN, Melby (DK)

(21) Appl. No.: 17/795,428

(22) PCT Filed: Jan. 29, 2021
(86) PCT No.: PCT/EP2021/052132

§ 371 Date: Jul. 26, 2022

Related U.S. Application Data
(60) Provisional application No. 62/968,710, filed on Jan. 31, 2020.

(51) Int. Cl. A61K 31/427; C12Q 1/6886; A61K 31/7068; A61P 35/00

(52) U.S. Cl. CPC A61K 31/427; C12Q 1/6886; A61K 31/7068; A61P 35/00; C12Q 2600/106; C12Q 2600/158; C12Q 1/6851

(57) ABSTRACT

Featured are methods, devices, and kits for detecting gene expression in a patient with a cancer and methods for determining responsiveness of a patient with a cancer to a treatment, such as treatment with ixabepilone or a pharmaceutically acceptable salt thereof. Also disclosed are methods of treating a patient with a cancer by administering a treatment, e.g., treatment with ixabepilone or a pharmaceutically acceptable salt thereof, in particular when the patient is determined to be responsive to the treatment based on the expression of the biomarkers described herein.

### SEQUENCE LISTING

The instant application contains a Sequence Listing which has been submitted electronically in ASCII format and is hereby incorporated by reference in its entirety. Said ASCII copy, created on Jan. 20, 2021 is named “51167-019WO2_Sequence_Listing_1.20.21_ST25.txt” and is 37,433 bytes in size.

### FIELD OF THE INVENTION

The use of biomarkers to predict the responsiveness of a cancer in a subject to a cancer therapy.

### BACKGROUND

DNA microarrays have been used to measure gene expression in tumor samples from patients and to facilitate diagnosis. Gene expression can reveal the presence of cancer in a patient in addition to the type, stage, and origin. Gene expression may even have a role in predicting the efficacy of cancer therapies. In recent decades, the National Cancer Institute (NCI) has tested cancer therapeutics for their effect in limiting the growth of 60 human cancer cell lines. The NCI has also measured gene expression in those 60 cancer cell lines using DNA microarrays. Various studies have explored the relationship between gene expression and therapeutic effect using the NCI datasets.

During cancer treatment, critical time is often lost due to a trial and error approach to finding an effective therapy. In addition, cancer cells often develop resistance to a previously effective therapy. In such situations, patient outcome would be greatly improved by early detection of such resistance.

Thus, there exists a need in the art for methods and devices that can predict the responsiveness of cancer patients to a medical treatment.

### SUMMARY OF THE INVENTION

Featured are methods for detecting gene expression of a biomarker (e.g., one, two, three, four, five, ten, fifteen, twenty, twenty five, or all of the biomarkers shown in Tables 1 and/or 2, such as HLA-DRA (SEQ ID NO: 1) and/or PLK2 (SEQ ID NO: 47), respectively, in a patient, such as a patient with a cancer (e.g., a patient with breast cancer or a recurrence thereof) to treatment with ixabepilone or a pharmaceutically acceptable salt thereof. Also featured are methods of treating cancer in a patient in need thereof (e.g., a patient with breast cancer or a recurrence thereof) that include administering ixabepilone or a pharmaceutically acceptable salt thereof to the patient, in which the patient is or has been determined to be responsive to ixabepilone or a pharmaceutically acceptable salt thereof according to the diagnostic methods described herein.

Exemplary types of cancer that can be diagnosed or treated with the methods include, e.g., breast cancer (e.g., estrogen receptor-positive (ER pos) breast cancer, metastatic form of breast cancer, or medullary carcinoma), ER-positive cancer, endometrial cancer (e.g., FGFR2-mutated or FGFR2-non-mutated advanced or metastatic endometrial cancer), renal cell carcinoma (RCC), hepatocellular carcinoma (HCC), gastrointestinal stromal tumor (GIST), lung cancer (e.g., non-small cell lung carcinoma, large cell carcinoma, bronchogenic carcinoma, and papillary adenocarcinoma), myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, prostate cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), ovarian cancer (e.g., ovarian adenocarcinoma or embryonal carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), bladder cancer, head and neck cancer (e.g., squamous cell carcinoma of the head and neck), metastatic cancer, oral cavity cancer, uterine cancer, testicular cancer (e.g., seminoma and embryonal carcinoma), skin cancer (e.g., squamous cell carcinoma, and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, liver cancer (e.g., hepatocellular carcinoma or hepatoma), larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, or neoplasms of the central nervous system. For example, the cancer may be a solid tumor (e.g., breast cancer) or a hematological cancer.

A first aspect features a method of determining responsiveness of a patient with a cancer (e.g., one of the cancers noted above, such as breast cancer) to ixabepilone or a pharmaceutically acceptable salt thereof. In particular, the patient may have recurrence of cancer, such as recurrence of breast cancer. The method includes: (a) contacting a sample (e.g., a tumor sample) from the patient including one or more nucleic acid molecules with a device (e.g., a microarray, such as a deoxyribonucleic acid (DNA)-based platform) including: (i) one or more single-stranded nucleic acid molecules capable of specifically hybridizing with the nucleotides of one or more biomarkers of sensitivity selected from the biomarkers of Table 1 (e.g., HLA-DRA (SEQ ID NO: 1)); and/or (ii) one or more single-stranded nucleic acid molecules capable of specifically hybridizing with the nucleotides of one or more biomarkers of resistance selected from the biomarkers of Table 2 (e.g., PLK2 (SEQ ID NO: 47)); and (b) measuring hybridization between the one or more nucleic acid molecules from the patient and the single-stranded nucleic acid molecules of the device to detect a level of expression of the one or more biomarkers of sensitivity and/or the one or more biomarkers of resistance. The patient is determined to be responsive to ixabepilone or a pharmaceutically acceptable salt thereof if: (i) the level of expression of the biomarker(s) of sensitivity (e.g., HLA-DRA (SEQ ID NO: 1)) is substantially similar to the level of expression of the biomarker(s) of sensitivity in a cell (e.g., a cancer cell) or tissue (e.g., a tumor tissue) known to be sensitive to ixabepilone or a pharmaceutically acceptable salt thereof (e.g., a tumor sample from a reference subject having the same diagnosis as the patient and that has been determined to be responsive to ixabepilone or a pharmaceutically acceptable salt thereof); (ii) the level of expression of the biomarker(s) of resistance (e.g., PLK2 (SEQ ID NO: 47)) is substantially similar to the level of expression of the biomarker(s) of resistance in a cell (e.g., a cancer cell) or tissue (e.g., a tumor tissue) known to be sensitive to ixabepilone or a pharmaceutically acceptable salt thereof (e.g., a tumor sample from a reference subject having the same diagnosis as the patient and that has been determined to be responsive to ixabepilone or a pharmaceutically acceptable salt thereof); (iii) the level of expression of the biomarker(s) of sensitivity (e.g., HLA-DRA (SEQ ID NO: 1)) is substantially dissimilar to the level of expression of the biomarker(s) of sensitivity in a cell (e.g., a cancer cell) or tissue (e.g., a tumor tissue) known to be resistant to ixabepilone or a pharmaceutically acceptable salt thereof (e.g., a tumor sample from a reference subject having the same diagnosis as the patient and that has been determined to be resistant to ixabepilone or a pharmaceutically acceptable salt thereof); and/or (iv) the level of expression of the biomarker(s) of resistance (e.g., PLK2 (SEQ ID NO: 47)) is substantially dissimilar to the level of expression of the biomarker(s) of resistance in a cell (e.g., a cancer cell) or tissue (e.g., a tumor tissue) known to be resistant to ixabepilone or a pharmaceutically acceptable salt thereof (e.g., a tumor sample from a reference subject having the same diagnosis as the patient and that has been determined to be resistant to ixabepilone or a pharmaceutically acceptable salt thereof). For example, a patient may be deemed sensitive to treatment with ixabepilone or a pharmaceutically acceptable salt thereof if the expression level of the biomarker(s) of sensitivity in a sample is above a cutoff value of the 50^{th} percentile in a cell or tissue obtained from a reference subject in a reference population known to be responsive to ixabepilone and having the same diagnosis as the patient, or greater (e.g., 60^{th} percentile, 70^{th} percentile, 80^{th} percentile, or 90^{th} percentile, or greater). As another example, a patient may be deemed sensitive to treatment with ixabepilone or a pharmaceutically acceptable salt thereof if the expression level of the biomarker(s) of resistance in a cell is above a cutoff value of the 50^{th} percentile in a cell or tissue obtained from a reference subject in a reference population known to be resistant to ixabepilone and having the same diagnosis as the patient, or greater (e.g., 60^{th} percentile, 70^{th} percentile, 80^{th} percentile, or 90^{th} percentile, or greater). As yet another example, a patient may be deemed sensitive to treatment with ixabepilone or a pharmaceutically acceptable salt thereof if the expression level of the biomarker(s) of sensitivity in a cell is below a cutoff value of the 50^{th} percentile in a cell or tissue obtained from a reference subject in a reference population resistant to ixabepilone treatment and having the same diagnosis as the patient, or less (e.g., 40^{th} percentile, 30^{th} percentile, 20^{th} percentile, or 10^{th} percentile, or less). In an additional example, a patient may be deemed sensitive to treatment with ixabepilone or a pharmaceutically acceptable salt thereof if the expression level of the biomarker(s) of resistance in a cell is below a cutoff value of the 50^{th} percentile in a cell or tissue obtained from a reference subject in a reference population known to be resistant to ixabepilone and having the same diagnosis as the patient, or less (e.g., 40^{th} percentile, 300^{th} percentile, 20^{th} percentile, or 100^{th} percentile, or less). Responsiveness of the patient to ixabepilone or a pharmaceutically acceptable salt thereof can also be assessed by calculating a difference score for the patient (mean of expression of the biomarkers of sensitivity noted above minus the mean of expression of the biomarkers of resistance noted above).

The method of the first aspect can further include administering ixabepilone or a pharmaceutically acceptable salt thereof to the patient having: (i) a level of expression of the biomarker(s) of sensitivity (e.g., HLA-DRA (SEQ ID NO: 1)) that is substantially similar to the level of expression of the biomarker(s) of sensitivity in a cell (e.g., a cancer cell) or tissue (e.g., a tumor tissue) known to be sensitive to ixabepilone or a pharmaceutically acceptable salt thereof; (ii) a level of expression of the biomarker(s) of resistance (e.g., PLK2 (SEQ ID NO: 47)) that is substantially similar to the level of expression of the biomarker(s) of resistance in a cell (e.g., a cancer cell) or tissue (e.g., a tumor tissue) known to be sensitive to ixabepilone or a pharmaceutically acceptable salt thereof; (iii) a level of expression of the biomarker(s) of sensitivity (e.g., HLA-DRA (SEQ ID NO: 1)) that is substantially dissimilar to the level of expression of the biomarker(s) of sensitivity in a cell (e.g., a cancer cell) or tissue (e.g., a tumor tissue) known to be resistant to ixabepilone or a pharmaceutically acceptable salt thereof; and/or (iv) a level of expression of the biomarker(s) of resistance (e.g., PLK2 (SEQ ID NO: 47)) that is substantially dissimilar to the level of expression of the biomarker(s) of resistance in a cell (e.g., a cancer cell) or tissue (e.g., a tumor tissue) known to be resistant to ixabepilone or a pharmaceutically acceptable salt thereof. The method can further include administering one or more cancer therapies other than ixabepilone or a pharmaceutically acceptable salt thereof to the patient having: (i) a level of expression of the biomarker(s) of sensitivity (e.g., HLA-DRA (SEQ ID NO: 1)) that is substantially dissimilar to the level of expression of the biomarker(s) of sensitivity in a cell (e.g., a cancer cell) or tissue (e.g., a tumor tissue) known to be sensitive to ixabepilone or a pharmaceutically acceptable salt thereof; (ii) a level of expression of the biomarker(s) of resistance (e.g., PLK2 (SEQ ID NO: 47)) that is substantially dissimilar to the level of expression of the biomarker(s) of resistance in a cell (e.g., a cancer cell) or tissue (e.g., a tumor tissue) known to be sensitive to ixabepilone or a pharmaceutically acceptable salt thereof; (iii) a level of expression of the biomarker(s) of sensitivity (e.g., HLA-DRA (SEQ ID NO: 1)) that is substantially similar to the level of expression of the biomarker(s) of sensitivity in a cell (e.g., a cancer cell) or tissue (e.g., a tumor tissue) known to be resistant to ixabepilone or a pharmaceutically acceptable salt thereof; and/or (iv) a level of expression of the biomarker(s) of resistance (e.g., PLK2 (SEQ ID NO: 47)) that is substantially similar to the level of expression of the biomarker(s) of resistance in a cell (e.g., a cancer cell) or tissue (e.g., a tumor tissue) known to be resistant to ixabepilone or a pharmaceutically acceptable salt thereof. In particular, the one or more cancer therapies can include surgery, radiation, or a therapeutic agent, such as capecitabine, a histone deacetylase (HDAC) inhibitor, an immune checkpoint inhibitor (e.g., a PD1 inhibitor, a PD-L1 inhibitor, or a CTLA-4 inhibitor), ipilimumab, a cyclin-dependent kinase inhibitor (e.g., a CDK inhibitor selective for CDK4 and CDK6, such as palbociclib (IBRANCE®) and abemaciclib (VERZENIO®, VERZENIOS®)), venetoclax (VENCLEXTA®, VENCLYXTO®), ibrutinib (IMBRUVICA®), bortezomib, carfilzomib, thalidomide, lenalidomide, pomalidomide, prednisone, dexamethasone, cyclophosphamide, vincristine, doxorubicin, melphalan, tegafur, irinotecan, oxaliplatin, cetuximab, leucovorin, SN-38, everolimus, temsirolimus, bleomycin, lomustine, depsipeptide, carboplatin, erlotinib, gemcitabine, mitoxantrone, cisplatin, busulfan, epirubicin, arsenic trioxide, bendamustine, fulvestrant, teniposide, adriamycin, decitabine, estramustine, etoposide, azaguanine, aclarubicin, mitoxantrone, mitomycin, paclitaxel, taxotere, Irofulven, 5-FU, ara-c, methylprednisolone, methotrexate, methyl-gag, belinostat, carboplatin, idarubicin, IL4-PR38, valproic acid, all-trans retinoic acid (ATRA), cytoxan, topotecan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, tamoxifen, floxuridine, thioguanine, PSC 833, herceptin, bevacizumab, celecoxib, iressa, anastrozole, letrozole, or rituximab. In particular embodiments, the additional therapeutic agent is capecitabine.

Also featured is a method of treating cancer in a patient in need thereof (e.g., a patient with one of the cancers noted above, such as breast cancer, that includes administering ixabepilone or a pharmaceutically acceptable salt thereof to the patient, in which the patient has been determined to be responsive to ixabepilone or a pharmaceutically acceptable salt thereof according to the method of the first aspect of the invention. In particular, the patient may have recurrence of cancer, such as recurrence of breast cancer.

A second aspect features a method of treating a patient having cancer (e.g., one of the cancers noted above, such as breast cancer). In particular, the patient may have recurrence of cancer, such as recurrence of breast cancer. The method includes: (a) contacting a sample (e.g., a tumor sample) from the patient including one or more nucleic acid molecules with a device including: (i) one or more single-stranded nucleic acid molecules capable of specifically hybridizing with the nucleotides of one or more biomarkers of sensitivity selected from the biomarkers of Table 1 (e.g., HLA-DRA (SEQ ID NO: 1)); and/or (ii) one or more single-stranded nucleic acid molecules capable of specifically hybridizing with the nucleotides of one or more biomarkers of resistance selected from the biomarkers of Table 2 (e.g., PLK2 (SEQ ID NO: 47)); (b) measuring hybridization between the one or more nucleic acid molecules from the patient and the single-stranded nucleic acid molecules of the device to detect a level of expression of the one or more biomarkers of sensitivity and/or the one or more biomarkers of resistance; and (c) administering ixabepilone or a pharmaceutically acceptable salt thereof to the patient. Ixabepilone or a pharmaceutically acceptable salt thereof can be administered to the patient if: (i) the level of expression of the biomarker(s) of sensitivity (e.g., HLA-DRA (SEQ ID NO: 1)) is substantially similar to the level of expression of the biomarker(s) of sensitivity in a cell (e.g., a cancer cell) or tissue (e.g., a tumor tissue) known to be sensitive to ixabepilone or a pharmaceutically acceptable salt thereof; (ii) the level of expression of the biomarker(s) of resistance (e.g., PLK2 (SEQ ID NO: 47)) is substantially similar to the level of expression of the biomarker(s) of resistance in a cell (e.g., a cancer cell) or tissue (e.g., a tumor tissue) known to be sensitive to ixabepilone or a pharmaceutically acceptable salt thereof; (iii) the level of expression of the biomarker(s) of sensitivity (e.g., HLA-DRA (SEQ ID NO: 1)) is substantially dissimilar to the level of expression of the biomarker(s) of sensitivity in a cell (e.g., a cancer cell) or tissue (e.g., a tumor tissue) known to be resistant to ixabepilone or a pharmaceutically acceptable salt thereof; and/or (iv) the level of expression of the biomarker(s) of resistance (e.g., PLK2 (SEQ ID NO: 47)) is substantially dissimilar to the level of expression of the biomarker(s) of resistance in a cell (e.g., a cancer cell) or tissue (e.g., a tumor tissue) known to be resistant to ixabepilone or a pharmaceutically acceptable salt thereof.

The method of the foregoing aspects may further include administering one or more additional therapies (e.g., surgery, radiation, or a therapeutic agent) to the patient prior to, concurrently with, or after administration of ixabepilone or a pharmaceutically acceptable salt thereof. In some embodiments, a therapeutic agent that is administered to the patient prior to, concurrently with, or after administration of ixabepilone or a pharmaceutically acceptable salt thereof is capecitabine. In some embodiments, a therapeutic agent that is administered to the patient prior to, concurrently with, or after administration of ixabepilone or a pharmaceutically acceptable salt thereof is capecitabine. In particular embodiments, a therapeutic agent that is administered to the patient prior to, concurrently with, or after administration of ixabepilone or a pharmaceutically acceptable salt thereof is one or more of capecitabine, an HDAC inhibitor, an immune checkpoint inhibitor (e.g., a PD1 inhibitor (e.g., Pembrolizumab, Nivolumab, and Cemiplimab), a PD-L1 inhibitor (e.g., Atezolizumab, Avelumab, and Durvalumab), and a CTLA-4 inhibitor (e.g., Ipilimumab, and Tremelimumab)), an aromatase inhibitor (e.g., a non-selective aromatase inhibitor, such as Aminoglutethimide and Testolactone; a selective aromatase inhibitor, such as anastrozole, letrozole, exemestane, vorozole, formestane, and fadrozole; and other aromatase inhibitors, such as 1,4,6-Androstatrien-3,17-dione (ATD) and 4-Androstene-3,6,17-trione (6-OXO)), an antiestrogen (e.g., a selective estrogen receptor modulator (SERM) (e.g., tamoxifen, clomifene, and raloxifene), an estrogen receptor silent antagonist, and selective estrogen receptor degrader (SERD) (e.g., fulvestrant)), an antigonadotropin (e.g., gonadotropin-releasing hormone (GnRH) analogue, compounds acting on sex steroid hormone receptors (e.g., progestogens, androgens, and estrogens), and steroid synthesis inhibitors (e.g., danazol and gestrinone)), a cyclin-dependent kinase inhibitor (e.g., a CDK inhibitor selective for CDK4 and CDK6, such as palbociclib (IBRANCE®) and abemaciclib (VERZENIO®, VERZENIOS®)), venetoclax (VENCLEXTA®, VENCLYXTO®), ibrutinib (IMBRUVICA®), bortezomib, carfilzomib, thalidomide, lenalidomide, pomalidomide, prednisone, dexamethasone, cyclophosphamide, vincristine, doxorubicin, melphalan, tegafur, irinotecan, oxaliplatin, cetuximab, leucovorin, SN-38, everolimus, temsirolimus, bleomycin, lomustine, depsipeptide, carboplatin, erlotinib, gemcitabine, mitoxantrone, cisplatin, busulfan, epirubicin, arsenic trioxide, bendamustine, teniposide, adriamycin, decitabine, estramustine, etoposide, azaguanine, aclarubicin, mitoxantrone, mitomycin, paclitaxel, taxotere, Irofulven, 5-FU, ara-c, methylprednisolone, methotrexate, methyl-gag, belinostat, carboplatin, idarubicin, IL4-PR38, valproic acid, all-trans retinoic acid (ATRA), cytoxan, topotecan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, floxuridine, thioguanine, PSC 833, herceptin, bevacizumab, celecoxib, iressa, anastrozole, letrozole, or rituximab. The therapeutic agent can be administered parenterally (e.g. intravenously, intramuscularly, transdermally, intradermally, intra-arterially, intracranially, subcutaneously, intraorbitally, intraventricularly, intraspinally, intraperitoneally, or intranasally), enterally, or topically. In a particular embodiment, the method of any of the foregoing aspects includes administering capecitabine to the subject two or more times. In some embodiments, the method of any of the foregoing aspects includes administering capecitabine to the subject one or more times daily, weekly, every two weeks, every three weeks, or monthly. In some embodiments, the capecitabine is administered two times per day. In some embodiments, the method includes administering capecitabine to the subject one or more times every three weeks. In some embodiments, the capecitabine is administered to the subject two times per day on days 1-14 of a three-week treatment cycle. In some embodiments, the capecitabine is administered to the subject at a dose of 1000 mg/m². In some embodiments, the capecitabine is administered to the subject at a dose of about 2000-2500 mg (e.g., 2000 mg, 2100 mg, 2200 mg, 2300 mg, 2400 mg, 2500 mg). In some embodiments, the capecitabine is administered to the subject at a dose of 2000 mg. In some embodiments, the capecitabine is administered as an intravenous infusion or injection. In a particular embodiment, the capecitabine is administered to the subject as an intravenous infusion containing capecitabine at a dose of 1000 mg/m² two times per day on days 1-14 of a three-week treatment cycle.

According to the first or second aspect, ixabepilone or a pharmaceutically acceptable salt thereof may be administered parenterally (e.g. intravenously, intramuscularly, transdermally, intradermally, intra-arterially, intracranially, subcutaneously, intraorbitally, intraventricularly, intraspinally, intraperitoneally, or intranasally), enterally (e.g., orally), or topically. Preferably, ixabepilone or a pharmaceutically acceptable salt thereof is administered as an intravenous infusion or injection.

Ixabepilone or a pharmaceutically acceptable salt thereof may be administered to the patient one or more times, such as one or more times daily (e.g., once daily for up to six days), weekly, every two weeks, every three weeks, or monthly. Preferably, ixabepilone or a pharmaceutically acceptable salt thereof is administered one or more times every three weeks. In particular, ixabepilone or a pharmaceutically acceptable salt thereof is administered once every three weeks. In some embodiments, the ixabepilone or a pharmaceutically acceptable salt thereof is administered on day one of a three-week treatment cycle. The method may further include administering a second dose of ixabepilone or a pharmaceutically acceptable salt thereof to the subject (e.g., patient) two days, four days, six days, one week, two weeks, three weeks, four weeks, or five weeks after administration of a first dose of ixabepilone or a pharmaceutically acceptable salt thereof. Ixabepilone or a pharmaceutically acceptable salt thereof may be administered in a particular dosage form (e.g., intravenous infusion or injection). The dose administered may be about 40 mg/m² (e.g., 40 mg/m²). In particular, ixabepilone or a pharmaceutically acceptable salt thereof may be administered at a dose(s) of about 40-120 mg (e.g., about 40 mg, 50 mg, 60 mg, 70 mg, 80 mg, 90 mg, 100 mg, 110 mg, or 120 mg). In particular embodiments, ixabepilone or a pharmaceutically acceptable salt thereof may be administered at a dose of 80 mg. In some embodiments, ixabepilone or a pharmaceutically acceptable salt thereof is administered to the subject on day one of a three-week treatment cycle at a dose at or about 40 mg/m² delivered as an intravenous infusion or injection. In some embodiments, the ixabepilone or a pharmaceutically acceptable salt thereof is formulated for administration as a solution comprising ixabepilone at a concentration of at or about 0.2 mg/mL to 0.6 mg/mL.

In particular embodiments of the first and second aspects, the contacting step (a) and the measuring step (b) may occur prior to, concurrent to, or after administration of ixabepilone or a pharmaceutically acceptable salt thereof to the patient. Each of the contacting step (a) and the measuring step (b) may occur multiple times.

In any of the above aspects, the device (e.g., a microarray, such as a DNA-based platform) can include at least two, at least three, at least four, at least five, at least six, at least seven, at least eight, at least nine, at least ten, or more single-stranded nucleic acid molecules capable of specifically hybridizing with the nucleotides of one or more biomarkers of sensitivity selected from the biomarkers of Table 1 (e.g., HLA-DRA (SEQ ID NO: 1)); and/or at least two, at least three, at least four, at least five, at least six, at least seven, at least eight, at least nine, at least ten, or more single-stranded nucleic acid molecules capable of specifically hybridizing with the nucleotides of one or more biomarkers of resistance selected from the biomarkers of Table 2 (e.g., PLK2 (SEQ ID NO: 47)). For example, the device may have single-stranded nucleic acid molecule(s) having the sequence of or complementary to each of the biomarkers of sensitivity selected from the biomarkers of Table 1 and for each of the biomarkers of resistance selected from the biomarkers of Table 2 that are affixed to the device and can be used to detect the level of expression of the biomarkers, e.g., by hybridization. In particular, one or more of the single-stranded nucleic acid molecules of the device have a length in the range of 10 to 100 nucleotides in length (e.g., a length in the range of 20 to 60 nucleotides).

In any of the above aspects, the method may include converting the level of expression of one or more of the biomarkers of sensitivity (e.g., one, two, three, four, five, ten, fifteen, twenty, twenty five, or all of the biomarkers shown in Table 1 (e.g., the top one biomarker, the top two biomarkers, the top three biomarkers, the top four biomarkers, the top five biomarkers, the top ten biomarkers, the top fifteen biomarkers, the top twenty biomarkers, the top twenty five biomarkers, or all of the biomarkers shown in Table 1), such as HLA-DRA (SEQ ID NO: 1)) and/or one or more of the biomarkers of resistance (e.g., one, two, three, four, five, ten, fifteen, twenty, twenty five, or all of the biomarkers shown in Table 2 (e.g., the top one biomarker, the top two biomarkers, the top three biomarkers, the top four biomarkers, the top five biomarkers, the top ten biomarkers, the top fifteen biomarkers, the top twenty biomarkers, the top twenty five biomarkers, or all of the biomarkers shown in Table 2), such as PLK2 (SEQ ID NO: 47)) into a mean score, in which the mean score indicates the responsiveness of the patient to ixabepilone or a pharmaceutically acceptable salt thereof. The method can further include subtracting the mean score for one or more of the biomarkers of resistance (e.g., one, two, three, four, five, ten, fifteen, twenty, twenty five, or all of the biomarkers shown in Table 2 (e.g., the top one biomarker, the top two biomarkers, the top three biomarkers, the top four biomarkers, the top five biomarkers, the top ten biomarkers, the top fifteen biomarkers, the top twenty biomarkers, the top twenty five biomarkers, or all of the biomarkers shown in Table 2), such as PLK2 (SEQ ID NO: 47)) from the mean score for one or more of the biomarkers of sensitivity (e.g., one, two, three, four, five, ten, fifteen, twenty, twenty five, or all of the biomarkers shown in Table 1 (e.g., the top one biomarker, the top two biomarkers, the top three biomarkers, the top four biomarkers, the top five biomarkers, the top ten biomarkers, the top fifteen biomarkers, the top twenty biomarkers, the top twenty five biomarkers, or all of the biomarkers shown in Table 1), such as HLA-DRA (SEQ ID NO: 1)) to obtain a difference score, in which the difference score indicates the responsiveness of the patient to ixabepilone. In particular, the mean score and/or the difference score of the biomarkers from a subject can be compared to the mean scores or difference scores of the biomarkers obtained from a reference population of tumor samples of the same type (e.g., from subjects diagnosed with the same type of tumor), in which the mean score or difference score of the biomarkers obtained from the subject falling at or above the 50^{th} percentile of the reference population, or the 60^{th} percentile, or the 70^{th} percentile, or the 80^{th} percentile, or the 90^{th} percentile, or greater, can be used to predict the likelihood that a tumor (or a subject from whom the tumor sample is taken) will be responsive to a treatment (e.g., treatment with ixabepilone or a pharmaceutically acceptable salt thereof). Alternatively, a mean score or difference score of the biomarkers obtained from the subject that falls below the 50^{th} percentile of the reference population can be used to predict the likelihood that a tumor (or a subject from whom the tumor sample is taken) will be non-responsive to a treatment (e.g., treatment with ixabepilone or a pharmaceutically acceptable salt thereof), For example, an expression level (or the mean score and/or the difference score thereof) of a sample (e.g., a tumor sample from a subject) at the 50^{th} percentile of the reference population, or the 60^{th} percentile, or the 70^{th} percentile, or the 80^{th} percentile, or the 90^{th} percentile, or greater, indicates that the sample (or the subject from whom the sample was taken) is predicted to be responsive to treatment with ixabepilone or a pharmaceutically acceptable salt thereof. The confidence of the prediction increases as the percentile level increases (e.g., an expression level above the 90^{th} percentile of a reference population indicates a greater likelihood of treatment responsiveness than an expression level at the 50^{th} percentile). Conversely, an expression level in the tested sample of below the 50^{th} percentile of the reference population indicates that the sample (or the subject from whom the sample was taken) is predicted to be non-responsive to treatment with ixabepilone or a pharmaceutically acceptable salt thereof.

In any of the above aspects, the device can be a microarray, such as a DNA-based platform. The expression level of the biomarkers of sensitivity (e.g., one, two, three, four, five, ten, fifteen, twenty, twenty five, or all of the biomarkers shown in Table 1 (e.g., the top one biomarker, the top two biomarkers, the top three biomarkers, the top four biomarkers, the top five biomarkers, the top ten biomarkers, the top fifteen biomarkers, the top twenty biomarkers, the top twenty five biomarkers, or all of the biomarkers shown in Table 1), such as HLA-DRA (SEQ ID NO: 1)) and/or the biomarkers of resistance (e.g., one, two, three, four, five, ten, fifteen, twenty, twenty five, or all of the biomarkers shown in Table 2 (e.g., the top one biomarker, the top two biomarkers, the top three biomarkers, the top four biomarkers, the top five biomarkers, the top ten biomarkers, the top fifteen biomarkers, the top twenty biomarkers, the top twenty five biomarkers, or all of the biomarkers shown in Table 2), such as PLK2 (SEQ ID NO: 47)) can be measured using microarray analysis or nucleic acid amplification methods (e.g., reverse transcription quantitative real-time polymerase chain reaction (RT-qPCR)). In particular, the level of expression of the biomarkers of sensitivity and/or the biomarkers of resistance is determined by detecting the level of mRNA transcribed from a gene coding one or more of the biomarkers of Table(s) 1 and/or 2.

In any of the above aspects, the biomarker of sensitivity may be selected from one or more of HLA-DRA (SEQ ID NOs: 1 and 2), ICAM3 (SEQ ID NO: 3), ITGB7 (SEQ ID NO: 4), CD8B (SEQ ID NO: 5), CD74 (SEQ ID NO: 6), HLA-DRB1 HLA-DRB4 HLA-DRB5 LOC100507709 LOC100507714 (SEQ ID NO: 7), IGJ (SEQ ID NO: 8), HLA-DRB1 HLA-DRB3 HLA-DRB4 LOC100507709 LOC100507714 (SEQ ID NO: 9), HLA-DPA1 (SEQ ID NOs: 10 and 12), HLA-DRB1 LOC100507709 LOC100507714 (SEQ ID NO: 11), CD28 (SEQ ID NO: 13), CD37 (SEQ ID NO: 14), NHP2 (SEQ ID NO: 15), MZB1 (SEQ ID NO: 16), ADCK3 (SEQ ID NO: 17), MYC (SEQ ID NO: 18), MEF2C (SEQ ID NOs: 19 and 24), RNASE6 (SEQ ID NO: 20), SRM (SEQ ID NO: 21), HAPLN1 (SEQ ID NO: 22), CYTIP (SEQ ID NO: 23), EIF3C EIF3CL (SEQ ID NO: 25), IQGAP2 (SEQ ID NO: 26), WBSCR22 (SEQ ID NO: 27), PIM2 (SEQ ID NO: 28), NCKAP1L (SEQ ID NO: 29), HCLS1 (SEQ ID NO: 30), MAGEA9 MAGEA9B (SEQ ID NO: 31), CTSS (SEQ ID NO: 32), GTF3A (SEQ ID NOs: 33 and 37), LCP1 (SEQ ID NO: 34), FAIM3 (SEQ ID NO: 35), HLA-DMB (SEQ ID NO: 36), RTP4 (SEQ ID NO: 38), MXI1 (SEQ ID NO: 39), SELPLG (SEQ ID NO: 40), MORA (SEQ ID NO: 41), POLR2H (SEQ ID NO: 42), BZW2 (SEQ ID NO: 43), CCDC88C (SEQ ID NO: 44), CORO1A (SEQ ID NO: 45), and AKAP1 (SEQ ID NO: 46).

In any of the above aspects, the biomarker of resistance may be selected from one or more of PLK2 (SEQ ID NO: 47), PLXNB2 (SEQ ID NO: 48), RRAS2 (SEQ ID NO: 49 and 50), PTPLA (SEQ ID NO: 51), P2RX5-TAX1 BP3 TAX1BP3 (SEQ ID NO: 52), STAT3 (SEQ ID NO: 53), PPIC (SEQ ID NO: 54), PTRF (SEQ ID NO: 55), RCN1 (SEQ ID NO: 56), ZFP36L1 (SEQ ID NO: 57), GFPT1 (SEQ ID NO: 58 AND 112), ACTN1 (SEQ ID NOs: 59 and 61), SEPT10 (SEQ ID NO: 60), COL4A1 (SEQ ID NO: 62), ERBB2IP (SEQ ID NO: 63), NNMT (SEQ ID NOs: 64 and 66), ADAM9 (SEQ ID NO: 65), TOR1AIP1 (SEQ ID NO: 67), ATP1B1 (SEQ ID NO: 68), CEBPD (SEQ ID NO: 69), FLII (SEQ ID NO: 70), FHL2 (SEQ ID NO: 71), SHC1 (SEQ ID NO: 72), SPTAN1 (SEQ ID NO: 73), CD81 (SEQ ID NO: 74), IQGAP1 (SEQ ID NO: 75), TGIF1 (SEQ ID NO: 76), PHACTR2 (SEQ ID NO: 77), COL4A2 (SEQ ID NO: 78), CEP55 (SEQ ID NO: 79), ABCC1 (SEQ ID NO: 80), PRSS23 (SEQ ID NO: 81), PTRF (SEQ ID NO: 82), TJP1 (SEQ ID NO: 83), CRK (SEQ ID NO: 84), LASP1 (SEQ ID NO: 85), PRC1 (SEQ ID NO: 86), TMEM189 TMEM189-UBE2V1 UBE2V1 (SEQ ID NO: 87), JAK1 (SEQ ID NO: 88), ACTN4 (SEQ ID NO: 89), FAM45A FAM45B (SEQ ID NO: 90), BIN1 (SEQ ID NOs: 91, 99, and 132), PPP2CB (SEQ ID NO: 92), EGFR (SEQ ID NO: 93), CNN3 (SEQ ID NO: 94), ARL6IP1 (SEQ ID NO: 95), FAIM (SEQ ID NO: 96), CDC20 (SEQ ID NO: 97), KIF23 (SEQ ID NO: 98), SDC4 (SEQ ID NO: 100), EZR (SEQ ID NO: 101), FGFR1 (SEQ ID NO: 102), LIMA1 (SEQ ID NO: 103), ITGA3 (SEQ ID NO: 104), TUBB6 (SEQ ID NO: 105), DYNLT1 (SEQ ID NO: 106), KRT7 (SEQ ID NO: 107), ANXA3 (SEQ ID NO: 108), MAPK1 (SEQ ID NO: 109), DOCK9 (SEQ ID NO: 110), ZMYM6 ZMYM6NB (SEQ ID NO: 111), MAP7D1 (SEQ ID NO: 113), BID (SEQ ID NO: 114), NMT1 (SEQ ID NO: 115), TRAM1 (SEQ ID NOs: 116 and 130), GPRC5A (SEQ ID NOS: 117 AND 166), MICALL1 (SEQ ID NO: 118), WDR1 (SEQ ID NO: 119), TRAF4 (SEQ ID NO: 120), AMOTL2 (SEQ ID NO: 121), AGRN (SEQ ID NO: 122), OBSL1 (SEQ ID NO: 123), LAPTM4B (SEQ ID NOS: 124 AND 159), MGAT4B (SEQ ID NO: 125), IQGAP1 (SEQ ID NO: 126), CTBP2 (SEQ ID NO: 127), AKR1B1 (SEQ ID NO: 128), CAPN2 (SEQ ID NO: 129), LACTB2 (SEQ ID NO: 131), PTPRK (SEQ ID NO: 133), CD24 (SEQ ID NOs: 134, 135, 144, and 157), PDLIM7 (SEQ ID NO: 136), ZNF238 (SEQ ID NO: 137), FAM129A (SEQ ID NO: 138), FAT1 (SEQ ID NO: 139), PPP4R1 (SEQ ID NO: 140), TPX2 (SEQ ID NO: 141), SOGA2 (SEQ ID NO: 142), FNTA (SEQ ID NO: 143), FNDC3B (SEQ ID NO: 145), SLC3A2 (SEQ ID NO: 146), S100A10 (SEQ ID NO: 147), RPA1 (SEQ ID NO: 148), HOXB2 (SEQ ID NO: 149), PI4KA PI4KAP1 PI4KAP2 (SEQ ID NO: 150), TRAF3 (SEQ ID NO: 151), BHLHE40 (SEQ ID NO: 152), CAV2 (SEQ ID NOs: 153 and 187), EPB41L2 (SEQ ID NO: 154), HDGFRP3 (SEQ ID NO: 155), AMIGO2 (SEQ ID NO: 156), CARD10 (SEQ ID NO: 158), LAPTM4B (SEQ ID NO: 159), MICA (SEQ ID NO: 160), TNFRSF10B (SEQ ID NO: 161), ZDHHC7 (SEQ ID NO: 162), SLC25A1 (SEQ ID NO: 163), ARPC5 (SEQ ID NO: 164), NF1 (SEQ ID NO: 165), GPRC5A (SEQ ID NO: 166), ACTB ACTB LOC100505829 (SEQ ID NO: 167), KRT8 (SEQ ID NO: 168), MKI67 (SEQ ID NO: 169), ANXA4 (SEQ ID NO: 170), COTL1 (SEQ ID NO: 171), LGALS3BP (SEQ ID NO: 172), PLOD2 (SEQ ID NO: 173), MPZL1 (SEQ ID NO: 174), RND3 (SEQ ID NO: 175), FZD6 (SEQ ID NO: 176), LIF (SEQ ID NO: 177), TNFRSF1A (SEQ ID NO: 178), AURKA (SEQ ID NO: 179), NR2F2 (SEQ ID NOs: 180 and 186), COPB1 (SEQ ID NO: 181), CD44 (SEQ ID NOs: 182 and 193), SMAD3 (SEQ ID NO: 183), CLPTM1 (SEQ ID NO: 184), LPHN2 (SEQ ID NO: 185), SGCE (SEQ ID NO: 188), FAM134B (SEQ ID NO: 189), IL6 (SEQ ID NO: 190), RAB32 (SEQ ID NO: 191), and FLNB (SEQ ID NO: 192).

In particular embodiments, the biomarkers of sensitivity may include one or more of: (a) SEQ ID NOs: 1-15. In more specific embodiments, the biomarker of sensitivity may be HLA-DRA (SEQ ID NO: 1).

In particular embodiments, the biomarkers of resistance may include one or more of: (a) SEQ ID NOs: 47-62. In more specific embodiments, the biomarker of resistance may be PLK2 (SEQ ID NO: 47).

In particular embodiments, the biomarker of sensitivity may be HLA-DRA (e.g., SEQ ID NO: 1) and the biomarker of resistance may be PLK2 (e.g., SEQ ID NO: 47).

In particular embodiments, the biomarkers of sensitivity may be selected from at least 5, at least 10, at least 15, at least 20, at least 25, or at least 27 of the biomarkers of Table 1 (e.g., at least the top 5 biomarkers, at least the top 10 biomarkers, at least the top 15 biomarkers, at least the top 20 biomarkers, at least the top 25 biomarkers, or at least the top 27 biomarkers of Table 1). The biomarkers of resistance may be selected from at least 5, at least 10, at least 15, at least 20, at least 25, or at least 27 of the biomarkers of Table 2 (e.g., at least the top 5 biomarkers, at least the top 10 biomarkers, at least the top 15 biomarkers, at least the top 20 biomarkers, at least the top 25 biomarkers, or at least the top 27 biomarkers of Table 2).

In any of the above aspects, the cancer is selected from a solid tumor cancer and a hematological cancer. For example, the cancer is, e.g., breast cancer, multiple myeloma, acute myelogenous leukemia (AML), acute lympho-blastic leukemia (ALL), chronic lymphocytic leukemia (CLL), myelodysplastic syndrome (MDS), chronic myelogenous leukemia-chronic phase (CMLCP), diffuse large B-cell lymphoma (DLBCL), cutaneous T-cell lymphoma (CTCL), peripheral T-cell lymphoma (PTCL), Hodgkin's lymphoma, hepatocellular carcinoma (HCC), cervical cancer, prostate cancer, kidney cancer, renal cell carcinoma (RCC), esophageal cancer, melanoma, glioma, pancreatic cancer, ovarian cancer, gastrointestinal stromal tumors (GIST), sarcoma, breast cancer, estrogen receptor-positive (ERpos) breast cancer, metastatic breast cancer, endometrial cancer, lung cancer, non-small cell lung carcinoma (NSCLC), mesothelioma, intestinal cancer, colon cancer, bladder cancer, adrenal cancer, gallbladder cancer, or squamous cell carcinoma of the head and neck (SCCHN). In some embodiments, the cancer is breast cancer (e.g., estrogen receptor-positive (ER pos) breast cancer, a metastatic form of breast cancer, or medullary carcinoma).

### Definitions

As used herein, “a” or “an” means “at least one” or “one or more” unless otherwise indicated. In addition, the singular forms “a”, “an”, and “the” include plural referents unless the context clearly dictates otherwise.

As used herein, “about” refers to an amount ±10% of the recited value.

As used herein, the terms “ixabepilone,” “azaepothilone B,” and “BMS-247550” refer to (1S,3S,7S,10R,11S,12S,16R)-7,11 dihydroxy-8,8,10,12,16-pentamethyl-3-[(1E)-1-methyl-2-(2-methyl-4-thiazolyl)ethenyl] 17-oxa-4-azabicyclo[14.1.0] heptadecane-5,9-dione or a tautomer thereof, or a mixture of tautomers thereof. Ixabepilone is a semi-synthetic analog of epothilone B that directly binds to β-tubulin monomers of microtubules and reduces microtubule disassembly. This microtubule-stabilizing property of ixabepilone renders it a potent anti-tumor agent by creating defects in mitotic spindle assembly, chromosome segregation, and cell division, thereby inhibiting the progression of mitosis and ultimately triggering apoptosis or reversion to the GO-phase of the cell cycle without cell division. As used herein, ixabepilone may refer to a pharmaceutically acceptable salt thereof. Ixabepilone has the following structure:

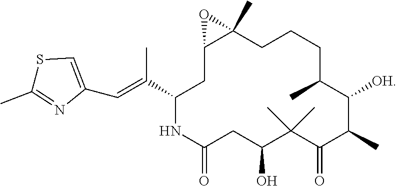

Exemplary methods of preparing ixabepilone are described in detail in US 2002/0188014 and US 2003/0004338, the disclosures of which are incorporated herein in their entirety.
The ixabepilone or a pharmaceutically acceptable salt disclosed herein can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbent or eluant). That is, ixabepilone or a pharmaceutically acceptable salt thereof may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of ixabepilone or a pharmaceutically acceptable salt can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on 25 opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The ixabepilone or a pharmaceutically acceptable salt thereof herein may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of ixabepilone or a pharmaceutically acceptable salt thereof is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When ixabepilone or a pharmaceutically acceptable salt thereof is named or depicted by structure without indicating the stereochemistry, and the ixabepilone or a pharmaceutically acceptable salt thereof has at least one chiral center, it is to be understood that the name or structure encompasses either enantiomer of the ixabepilone or a pharmaceutically acceptable salt thereof free from the corresponding optical isomer, a racemic mixture of the ixabepilone or a pharmaceutically acceptable salt thereof, or mixtures enriched in one enantiomer relative to its corresponding optical isomer. When ixabepilone or a pharmaceutically acceptable salt thereof is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

By “biomarker” is meant a nucleic acid molecule (e.g., an mRNA or its complement, for example, a cDNA) or a protein encoded by the nucleic acid molecule present in, or from, a cell or tissue (e.g., tumor tissue). The expression of the biomarker correlates to the responsiveness (e.g., sensitivity or resistance) of the cell or tissue (and thus, the patient containing the cell or tissue or the patient from which the cell or tissue was obtained) to a cancer treatment (e.g., ixabepilone or a pharmaceutically acceptable salt thereof). In particular, a biomarker of sensitivity is a nucleic acid molecule (e.g., a mRNA or its complement) expressed from any one of the genes shown in Table 1, or the protein encoded by the nucleic acid molecule, and a biomarker of resistance is a nucleic acid molecule (e.g., a mRNA or its complement) expressed from any one of the genes shown in Table 2, or the protein encoded by the nucleic acid molecule.

The terms “cancer” and “cancerous” refer to or describe the physiological condition in mammals (e.g., humans) that is typically characterized by unregulated cell proliferation. Examples of cancer include, but are not limited to, breast cancer (e.g., medullary carcinoma), myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, prostate cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), ovarian cancer (e.g., ovarian adenocarcinoma or embryonal carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), ER-positive cancer, bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), metastatic cancer, oral cavity cancer, uterine cancer, testicular cancer (e.g., seminoma and embryonal carcinoma), skin cancer (e.g., squamous cell carcinoma and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, liver cancer (e.g., hepatocellular carcinoma or hepatoma), larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, and neoplasms of the central nervous system. The term cancer includes hematological cancers (e.g., cancer of the blood, such as multiple myeloma) and solid tumors (e.g., breast cancer).

The terms “expression level” and “level of expression,” as used herein interchangeably, refer to the amount of a gene product in a cell, tissue, biological sample, organism, or patient, e.g., amounts of DNA, RNA (e.g. messenger RNA (mRNA) of), or protein encoded by a given gene.

“Gene” as used herein indicates a coding or noncoding gene whose activity can be determined by measuring the produced RNA. Examples include protein coding genes, microRNAs, small nuclear RNAs and other RNAs with catalytic, regulatory or coding properties.

To “inhibit growth” as used herein means causing a reduction in cell growth (e.g., cancer cell growth, e.g., as compared to the growth inhibition of the NCI60 cancer cell lines as a reference) in vivo or in vitro by, e.g., 10%, 20%, 30%, 40%, 50%, 60%, 70%, 80%, 90%, 95%, or 99% or more, as evident by a reduction in the proliferation of cells exposed to a treatment (e.g., ixabepilone or a pharmaceutically acceptable salt thereof), relative to the proliferation of cells in the absence of the treatment. Growth inhibition may be the result of a treatment (e.g., ixabepilone or a pharmaceutically acceptable salt thereof) that induces apoptosis in a cell, induces necrosis in a cell, slows cell cycle progression, disrupts cellular metabolism, induces cell lysis, or induces some other mechanism that reduces the proliferation of cells.

“Microarray” as used herein means a device employed by any method that quantifies one or more subject oligonucleotides, e.g., RNA, DNA, cDNA, or analogues thereof, at a time. For example, many DNA microarrays, including those made by Affymetrix (e.g., an Affymetrix HG-U133A array), use several probes for determining the expression of a single gene. The DNA microarray may contain oligonucleotide probes that may be, e.g., full-length cDNAs complementary to an RNA or cDNA fragments that hybridize to part of an RNA. The DNA microarray may also contain modified versions of DNA or RNA, such as locked nucleic acids or LNA. Exemplary RNAs include mRNA, miRNA, and miRNA precursors.

As used herein, the term “percent (%) sequence identity” refers to the percentage of nucleic acid residues of a candidate sequence, e.g., a probe or primer of the invention, that are identical to the nucleic acid residues of a reference sequence, e.g., a biomarker sequence of the invention, after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using computer software, such as BLAST, BLAST-2, BLAST-P, BLAST-N, BLAST-X, WU-BLAST-2, ALIGN, ALIGN-2, CLUSTAL, Megalign (DNASTAR). In addition, those skilled in the art can determine appropriate parameters for measuring alignment, including any algorithms needed to achieve optimal alignment over the length of the sequences being compared.

“NCI60” as used herein means a panel of 60 cancer cell lines from lung, colon, breast, ovarian, leukemia, renal, melanoma, prostate and brain cancers including the following cancer cell lines: NSCLC_NCIH23, NSCLC_NCIH522, NSCLC_A549ATCC, NSCLC_EKVX, NSCLC_NCIH226, NSCLC_NCIH332M, NSCLC_H460, NSCLC_HOP62, NSCLC_HOP92, COLON_HT29, COLON_HCC-2998, COLON_HCT116, COLON_SW620, COLON_COLO205, COLON_HCT15, COLON_KM12, BREAST_MCF7, BREAST_MCF7ADRr, BREAST_MDAMB231, BREAST_HS578T, BREAST_MDAMB435, BREAST_MDN, BREAST_BT549, BREAST_T47D, OVAR_OVCAR3, OVAR_OVCAR4, OVAR_OVCAR5, OVAR_OVCAR8, OVAR_IGROV1, OVAR_SKOV3, LEUK_CCRFCEM, LEUK_K562, LEUK_MOLT4, LEUK_HL60, LEUK_RPM18266, LEUK_SR, RENAL_UO31, RENAL_SN12C, RENAL_A498, RENAL_CAKI1, RENAL_RXF393, RENAL_7860, RENAL_ACHN, RENAL_TK10, MELAN_LOXIMVI, MELAN_MALME3M, MELAN_SKMEL2, MELAN_SKMEL5, MELAN_SKMEL28, MELAN_M14, MELAN_UACC62, MELAN_UACC257, PROSTATE_PC3, PROSTATE_DU145, CNS_SNB19, CNS_SNB75, CNS_U251, CNS_SF268, CNS_SF295, and CNS_SF539.

The terms “patient” and “subject,” as used interchangeably herein, refer to any animal (e.g., a mammal, such as a human). A patient to be treated or tested for responsiveness to a treatment (e.g., ixabepilone or a pharmaceutically acceptable salt thereof) according to the methods described herein may be one who has been diagnosed with a cancer, such as breast cancer. Diagnosis may be performed by any method or techniques known in the art, such as x-ray, MRI, or biopsy, and confirmed by a physician. To minimize exposure of a patient to drug treatments that may not be therapeutic, the patient may be determined to be either responsive or non-responsive to a cancer treatment, such as ixabepilone or a pharmaceutically acceptable salt thereof, according to the methods described herein.

As used herein, the term “pharmaceutically acceptable salt” means any pharmaceutically acceptable salt of the compound of any of the compounds described herein. For example, pharmaceutically acceptable salts of ixabepilone or a pharmaceutically acceptable salt thereof described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The ixabepilone described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

“Resistance” as used herein means that a cell (e.g., a cancer cell) or a tissue (e.g., a tumor) in vitro or in vivo (e.g., in a subject with a cancer, such as a human) is tolerant to treatment with an anti-cancer agent (e.g., ixabepilone or a pharmaceutically acceptable salt thereof), e.g., the cell or tissue (e.g., tumor tissue) is able to survive and grow despite exposure to (e.g., treatment with) an anti-cancer agent (e.g., ixabepilone or a pharmaceutically acceptable salt thereof). Resistance may arise via exploitation by a cell or tissue (e.g., a tumor tissue) of one or more of drug inactivation, drug target alteration, drug efflux, DNA damage repair, cell death inhibition, cell cycle regulation, epithelial-mesenchymal transition (EMT), epigenetics, and other mechanisms. A “resistant” cell or tissue refers to a cell (e.g., a cancer cell) or a tissue (e.g., a tumor), respectively, in vitro or in vivo (e.g., in a subject with a cancer, such as a human) that has acquired and/or exhibits resistance to a treatment (e.g., ixabepilone or a pharmaceutically acceptable salt thereof). For example, a resistant cell or tissue (e.g., a tumor tissue) is one that, upon exposure of the cell (e.g., a cancer cell) or the tissue (e.g., a tumor), respectively, to a cancer therapeutic (e.g., ixabepilone or a pharmaceutically acceptable salt thereof) exhibits an inhibition in growth of the cell or tumor of less than 30%, 25%, 20%, 15%, 10%, 5%, or 1% relative to the growth of a cell or tissue not exposed to the treatment. Resistance to treatment may be determined by a cell proliferation assay, e.g., a cell-based assay, which measures the growth of treated cells as a function of the absorbance of the cells of an incident light beam, such as the NC160 assays described herein. In this assay, greater absorbance indicates greater cell growth, and thus, resistance to the treatment.

The terms “sensitivity” and “responsiveness,” as used herein, refer to the likelihood that a cancer treatment (e.g., ixabepilone or a pharmaceutically acceptable salt thereof) has (e.g., induces) a desired effect, or alternatively refers to the strength of a desired effect caused or induced by the treatment in a cell (e.g., a cancer cell) or a tissue (e.g., a tumor) in vitro or in vivo (e.g., in a subject with a cancer, such as a human). For example, the desired effect can include inhibition of the growth of a cell (e.g., a cancer cell) in vitro by more than 30%, 35%, 40%, 45%, 50%, 55%, 60%, 65%, 70%, 75%, 80%, 85%, 90%, 95%, or 100% relative to the growth of a cell (e.g., a cancer cell) not exposed to the treatment. The desired effect can also include reduction in tumor mass by, e.g., about 30%, 35%, 40%, 45%, 50%, 55%, 60%, 65%, 70%, 75%, 80%, 85%, 90%, 95%, or 100%. “Sensitive” and “responsive” as used herein refer to a cell (e.g., a cancer cell) or a tissue (e.g., a tumor) in vitro or in vivo (e.g., in a subject with a cancer, such as a human) that is responsive to exposure to a therapeutic (e.g., ixabepilone or a pharmaceutically acceptable salt thereof). Responsiveness to treatment may be determined by a cell proliferation assay, e.g., a cell-based assay, which measures the growth of treated cells as a function of the absorbance of the cells of an incident light beam, such as the NC160 assays described herein. In this assay, lesser absorbance indicates lesser cell growth, and thus, sensitivity or responsiveness to the treatment. A greater reduction in growth indicates more sensitivity or responsiveness to the treatment.

The term “sample,” as used herein, refers to any specimen (such as cells, tissue (e.g., a tissue sample obtained by biopsy), blood, serum, plasma, urine, cerebrospinal fluid, or pancreatic fluid) taken from a subject. Preferably, the sample is taken from a portion of the body affected by a cancer (e.g., a biopsy of the cancer tissue). Biopsy may involve fine needle aspiration biopsy, core needle biopsy (e.g., stereotactic core needle biopsy, vacuum-assisted core biopsy, or magnetic resonance imaging (MRI) guided biopsy), or surgical biopsy (e.g., incisional biopsy or excisional biopsy). The sample may undergo additional purification and processing, for example, to remove cell debris and other unwanted molecules. Additional processing may further involve amplification, e.g., using PCR (e.g., RT-PCR). The standard methods of sample purification, such as removal of unwanted molecules, are known in the art.

“Substantially similar” or “corresponds,” as used herein with respect to a numerical value of a parameter of one or more of the biomarker(s) of sensitivity and/or resistance (e.g., biomarker expression level, difference score, or mean score), e.g., as determined in a test sample (e.g., a tumor biopsy) from a cancer patient, means that the numerical value of the parameter in the test sample is ±0-30% of the numerical value of the parameter in a reference sample (e.g., a cell (e.g., a cancer cell) or tissue (e.g., a tumor), such as a cell or a tissue obtained from a subject having the same diagnosis as a subject from whom the test sample was obtained) known to be sensitive or resistant to ixabepilone or a pharmaceutically acceptable salt thereof). For example, a numerical value of a parameter in a test sample may be substantially similar to, or may correspond to, the numerical value of the parameter in a reference sample if the parameter values of the test and reference samples differ by, e.g., less than 30%, less than 29%, less than 28%, less than 27%, less than 26%, less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1%.

“Substantially dissimilar,” as used herein with respect to a numerical value of a parameter of one or more of the biomarker(s) of sensitivity and/or resistance (e.g., biomarker expression level, difference score, or mean score), e.g., as determined in a test sample (e.g., a tumor biopsy) from a cancer patient, means that the numerical value of the parameter in the test sample deviates by greater than 30% from the numerical value of the parameter in a reference sample (e.g., a cell (e.g., a cancer cell) or tissue (e.g., a tumor), such as a cell or a tissue obtained from a subject having the same diagnosis as a subject from whom the test sample was obtained) known to be sensitive or resistant to ixabepilone or a pharmaceutically acceptable salt thereof). For example, a numerical value of a parameter in a test sample may be substantially dissimilar to the numerical value of the parameter in a reference sample if the parameter values of the test and reference samples differ by, e.g., greater than 30%, 35%, 40%, 45%, 50%, 55%, 60%, 65%, 70%, 75%, 80%, 85%, 90%, 95%, 100% or more.

“Treatment,” “medical treatment,” to “treat,” and “therapy,” as used interchangeably herein, refer to administering or exposing a patient (e.g., a human) with a cancer (e.g., breast cancer), a cancer cell, or a tumor to an anti-cancer agent (e.g., a drug, a protein, an antibody, a nucleic acid, a chemotherapeutic agent, or a radioactive agent), or to some other form of medical intervention used to treat or prevent a disease, disorder, or condition (e.g., surgery, cryotherapy, radiation therapy, or combinations thereof). In particular, a medical treatment can include ixabepilone or a pharmaceutically acceptable salt thereof. For example, the cancer to be treated is a hematological cancer or a solid tumor. Examples of cancer include, e.g., breast cancer (e.g., medullary carcinoma), myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, prostate cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), ovarian cancer (e.g., ovarian adenocarcinoma or embryonal carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), ER-positive cancer, endometrial cancer, bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), metastatic cancer, oral cavity cancer, uterine cancer, testicular cancer (e.g., seminoma and embryonal carcinoma), skin cancer (e.g., squamous cell carcinoma, and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, liver cancer (e.g., hepatocellular carcinoma or hepatoma), larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, or neoplasms of the central nervous system. Radiation therapy includes the administration of a radioactive agent to a patient or exposure of a patient to radiation. The radiation may be generated from sources such as particle accelerators and related medical devices or agents that emit, e.g., X-radiation, gamma radiation, or electron (Beta radiation) beams. A treatment may be or further include surgery, e.g., to remove a tumor from a subject or living organism.

Other features and advantages of the invention will be apparent from the following Detailed Description, the drawings, and the claims.

### BRIEF DESCRIPTION OF THE DRAWINGS

FIG. 1 is a graph showing predicted ixabepilone sensitivity in pre-treatment biopsy samples from breast cancer patients later treated neoadjuvantly with ixabepilone as part of a clinical trial. Patients with pathological complete remission (pCR) are predicted to be more sensitive to ixabepilone than patients with no pCR (No_pCR). Pearson correlation between outcome (pCR=1; no pCR=0) and prediction score is 0.29 (p=0.0003, one-sided). A cutoff of, e.g., 50, separates most responders from non-responders.

FIG. 2 is a graph showing ixabepilone response rate (% pCR) as a function of DRP score cutoff.

### DETAILED DESCRIPTION OF THE INVENTION

We have discovered that the expression levels of the biomarkers shown in Tables 1 and/or 2 can be used to determine whether a subject with a cancer (e.g., a subject diagnosed with breast cancer) will likely be responsive to ixabepilone or a pharmaceutically acceptable salt thereof. A device, such as a microarray, with one or more single-stranded oligonucleotide probes that have substantial identity (e.g., at least 85%, 90%, 95%, 99%, or 100% sequence identity) to a sequence that is complementary or identical to the nucleic acid sequence of one or more (e.g., all) biomarkers shown in Tables 1 and/or 2 can be used according to the method described herein to assess the responsiveness of a cancer patient to treatment with ixabepilone or a pharmaceutically acceptable salt thereof. For example, the probes can be used to detect one or more (e.g., one, two, three, four, five, ten, fifteen, twenty, twenty five, or all) of the biomarkers of sensitivity listed in Table 1, such as HLA-DRA (SEQ ID NO: 1), in a sample (e.g., a tumor sample) from a patient having cancer. Additionally, the probes can be used to detect one or more (e.g., one, two, three, four, five, ten, fifteen, twenty, twenty five, or all) of the biomarkers of resistance listed in Table 2, such as PLK2 (SEQ ID NO: 47), in a sample (e.g., a tumor sample) from a patient having cancer. Accordingly, the invention features individual biomarkers (e.g., HLA-DRA (SEQ ID NO: 1) or PLK2 (SEQ ID NO: 47)) and sets of biomarkers shown in Tables 1 and/or 2 that can be used to determine the responsiveness of a cancer patient to ixabepilone or a pharmaceutically acceptable salt thereof at various stages of disease progression (e.g., patients diagnosed with cancer or patients after cancer recurrence) and at different times during the treatment process (e.g., prior to administration of any cancer treatment, after administration of one or more cancer treatments other than ixabepilone or a pharmaceutically acceptable salt thereof, prior to administration of ixabepilone or a pharmaceutically acceptable salt thereof, or during administration of ixabepilone or a pharmaceutically acceptable salt thereof).

The biomarkers of sensitivity and resistance to ixabepilone are described in detail in Tables 1 and 2, respectively, below.

TABLE 1
mRNA biomarkers of sensitivity to ixabepilone.
Affymetrix IDs refer to the array type HG-U133_Plus_2. Sequences are representative probes from a
probeset of typically 11 probe sequences. More than one probeset can target the same gene.

| | Affymetrix | | | SEQ |
|---|---|---|---|---|
| Gene | ID | Correlation | Representative Probe Sequence | ID NO: |
| HLA-DRA | 208894_at | 0.502 | CGATCACCAATGTACCTCCAGAGGT | 1 |
| HLA-DRA | 210982_s_at | 0.46 | TGGGAGTTTGATGCTCCAAGCCCTC | 2 |
| ICAM3 | 204949_at | 0.435 | ACCAACGGAGCGGCAGTTACCATGT | 3 |
| ITGB7 | 205718_at | 0.411 | TATGACCGCCGGGAATACAGTCGCT | 4 |
| CD8B | 207979_s_at | 0.399 | GGTCTTTCATGGGGTGATGCTGGGC | 5 |
| CD74 | 209619_at | 0.399 | ACACAAGGCTCCAAGACCTAGGCTC | 6 |
| HLA-DRB1 HLA- | 209312_x_at | 0.398 | GAGTGGTTTCTATCCAGGCAGCATT | 7 |
| DRB4 HLA-DRB5 | | | | |
| LOC100507709 | | | | |
| LOC100507714 | | | | |
| IGJ | 212592_at | 0.378 | GTATCAAAATCTTCCAATTATCATG | 8 |
| HLA-DRB1 HLA- | 215193_x_at | 0.372 | TCTGTGAGTGGTTTCTATCCAGGCA | 9 |
| DRB3 HLA-DRB4 | | | | |
| LOC100507709 | | | | |
| LOC100507714 | | | | |
| HLA-DPA1 | 211991_s_at | 0.366 | CCAGATGCCTGAGACAACGGAGACT | 10 |
| HLA-DRB1 | 208306_x_at | 0.356 | ACCTTCCAGACCCTGGTGATGCTGG | 11 |
| LOC100507709 | | | | |
| LOC100507714 | | | | |
| HLA-DPA1 | 211990_at | 0.355 | TCATTGAGCCTTTTATCCTCTGTTC | 12 |
| CD28 | 206545_at | 0.338 | GATGAATCACACTTGAGATGTTTCT | 13 |
| CD37 | 204192_at | 0.337 | AGCTCAGCAGGCTTGGACACCTGGC | 14 |
| NHP2 | 209104_s_at | 0.333 | GAAATTTGCCCTATGTCTATATCCC | 15 |
| MZB1 | 221286_s_at | 0.325 | GGGGGCCTGCTCAGAGAAGGTGTCA | 16 |
| ADCK3 | 218168_s_at | 0.323 | AGGCTCCTCACAGGCGCTGTGAATT | 17 |
| MYC | 202431_s_at | 0.316 | GCTAAAACGGAGCTTTTTTGCCCTG | 18 |
| MEF2C | 209200_at | 0.313 | GGAGCAATCCAAGCCACATATCTTC | 19 |
| RNASE6 | 213566_at | 0.313 | ATTTCTAACCCTGCAACTTTTGCCA | 20 |
| SRM | 201516_at | 0.31 | TCGCCCACCAACCAAGTGTTACAAG | 21 |
| HAPLN1 | 205523_at | 0.307 | GCTACTCAGAGCAACAGGTTCCACA | 22 |
| CYTIP | 209606_at | 0.305 | GTTGTGTTATAGTTTATGCTTCTTA | 23 |
| MEF2C | 209199_s_at | 0.302 | TGAACAATCCCGGTGTGTCAGGACC | 24 |
| EIF3C EIF3CL | 210949_s_at | 0.302 | ACTGTGCACTCCATCATCAGCAAAA | 25 |
| IQGAP2 | 203474_at | 0.3 | ACTGTGATATAGGTACTCTGATTTA | 26 |
| WBSCR22 | 207628_s_at | 0.296 | GTCACCACGCGGTTCTGGAAAGGCA | 27 |
| PIM2 | 204269_at | 0.295 | TATTGGGTCAAGCTGCTTACCTGCC | 28 |
| NCKAP1L | 209734_at | 0.289 | CCTGCTTCGAAATGCCTATCGGGAG | 29 |
| HCLS1 | 202957_at | 0.288 | GAGTGACTAGAGCTCACTGTCTACT | 30 |
| MAGEA9 | 210437_at | 0.285 | CATGTATACCTGGATTTGCTTGGCT | 31 |
| MAGEA9B | | | | |
| CTSS | 202902_s_at | 0.284 | TATGAAGCACTTTCTCTTAACTTAA | 32 |
| GTF3A | 201338_x_at | 0.283 | GATGTATGTCGCTGTCCAAGAGAAG | 33 |
| LCP1 | 208885_at | 0.282 | TGTGCCAATCAATAGCACCCCTACT | 34 |
| FAIM3 | 221602_s_at | 0.28 | GAAAGGAGGAAAGCCCTCTCCAGGC | 35 |
| HLA-DMB | 203932_at | 0.279 | GATCCTATGGTTTCTCCATCCAATT | 36 |
| GTF3A | 215091_s_at | 0.276 | GTCTCACTAGGCATGCTGTTGTACA | 37 |
| RTP4 | 219684_at | 0.276 | AAATAGGCTTGCCACTTTCTCTTAT | 38 |
| MXI1 | 202364_at | 0.275 | AGTTCAGCACTTGTCTCATTTTAAT | 39 |
| SELPLG | 209879_at | 0.273 | GTGACGGACTTCTGAGGCTGTTTCC | 40 |
| IL10RA | 204912_at | 0.269 | AGTACTCTTAGGTGCCAGTCTGGTA | 41 |
| POLR2H | 209302_at | 0.264 | CCTTCCAGGGCTGACCAGTTTGAGT | 42 |
| BZW2 | 217809_at | 0.263 | TTCAGGTACTGTGTGGTGACCGCCC | 43 |
| CCDC88C | 215343_at | 0.257 | GCAACTACAATGACTGTACTCTCTA | 44 |
| CORO1A | 209083_at | 0.256 | GTCGGACCTGTTCCAGGAGGACCTG | 45 |
| AKAP1 | 201675_at | 0.253 | TGAACTGACTAATTGGTATCCACTA | 46 |

Note that HLA-DRA is targeted by two different probesets (SEQ ID NOs: 1 and 2), HLA-DPA1 is targeted by two different probesets (SEQ ID NOs: 10 and 12), MEF2C is targeted by two different probesets (SEQ ID NOs: 19 and 24), and GTF3A is targeted by two different probesets (SEQ ID NOs: 33 and 37).

TABLE 2
mRNA biomarkers of resistance to ixabepilone.
Affymetrix IDs refer to the array type HG-U133_Plus_2. Sequences are representative probes
from a probeset of typically 11 probe sequences. More than one probeset can target the same gene.

| | Affymetrix | | | SEQ ID |
|---|---|---|---|---|
| Gene ID | ID | Correlation | Probe sequence | NO: |
| PLK2 | 201939_at | −0.455 | TCAGCCAGAGGACTTTGGAACTGTG | 47 |
| PLXNB2 | 208890_s_at | −0.443 | AGAAAAAGCGGTACGATGCCTTCCT | 48 |
| RRAS2 | 212589_at | −0.432 | GGTATTGCACAGTTGTCACTTTATC | 49 |
| RRAS2 | 212590_at | −0.431 | AATGATCACCATGTTAGCCTTAGAC | 50 |
| PTPLA | 219654_at | −0.427 | ATTTTCTTCTTATAACCATGGCATC | 51 |
| P2RX5-TAX1BP3 | 215464_s_at | −0.407 | CTGCTGTGACCACAGGCTCAGGTCC | 52 |
| TAX1BP3 | | | | |
| STAT3 | 208991_at | −0.406 | AGCTACATACTCCTGGCATTGCACT | 53 |
| PPIC | 204517_at | −0.405 | GAACTTAAATATATCCCCTTCCTCA | 54 |
| PTRF | 208789_at | −0.392 | GAAGGGAGTGTTGCTCCCAGTCCAG | 55 |
| RCN1 | 201063_at | −0.385 | CCCCGCAACAGCTTATACCATGGAA | 56 |
| ZFP36L1 | 211962_s_at | −0.381 | TCTTTAGGCCTTTCACAACTAGGAC | 57 |
| GFPT1 | 202722_s_at | −0.375 | GTTGGGTATCCTACTACTTTGTGTT | 58 |
| ACTN1 | 208637_x_at | −0.372 | AGGTGCTCTGGACTACATGTCCTTC | 59 |
| SEPT10 | 212698_s_at | −0.367 | ATAAAGTACCTTTGAGCATGAGTGT | 60 |
| ACTN1 | 208636_at | −0.363 | GACTAGTCTCTTTATCAGCACACAC | 61 |
| COL4A1 | 211980_at | −0.363 | CATTAGTATTCCTCATTCTGCATCC | 62 |
| ERBB2IP | 217941_s_at | −0.362 | ACACCAACCAACATTATTTTTGCAA | 63 |
| NNMT | 202238_s_at | −0.361 | GACTACTCAGACCAGAACCTGCAGG | 64 |
| ADAM9 | 202381_at | −0.359 | AAGCATGACATTCGTTCACAATAGC | 65 |
| NNMT | 202237_at | −0.357 | CTTTTCTCCCTGGTGGCGAGGAAGC | 66 |
| TOR1AIP1 | 212408_at | −0.355 | CTAGATCTATCCACCTTGTTTTTTT | 67 |
| ATP1B1 | 201242_s_at | −0.354 | AACCTACTAGTCTTGAACAAACTGT | 68 |
| CEBPD | 203973_s_at | −0.353 | GGACAGCAGACTGCCGGTAACGCGC | 69 |
| FLII | 212025_s_at | −0.352 | GACACCTCCTACAGCAAGCAGGTTA | 70 |
| FHL2 | 202949_s_at | −0.351 | CAGAGAGGGACGACATCCTGTGCCC | 71 |
| SHC1 | 214853_s_at | −0.351 | TGCAGTTCCTGAGTACCTTCTACAG | 72 |
| SPTAN1 | 215235_at | −0.351 | GATCTGTGTCTTGAAGCAGCTGCCC | 73 |
| CD81 | 200675_at | −0.349 | TTGTTCTGAACTTTCCTGTTACCTT | 74 |
| IQGAP1 | 210840_s_at | −0.349 | TGTGCCTTTATTTTATGAGCCCCAG | 75 |
| TGIF1 | 203313_s_at | −0.348 | GGACCTCAACCAGGACTTCAGTGGA | 76 |
| PHACTR2 | 204048_s_at | −0.348 | TTTTGATCTTTCCCTATCTTGATTA | 77 |
| COL4A2 | 211966_at | −0.346 | CCATCGCGGTCCACAGTCAGGATGT | 78 |
| CEP55 | 218542_at | −0.346 | TGAAACTTAACCCAGCTGTGTTCCC | 79 |
| ABCC1 | 202804_at | −0.345 | AATAGTGAGAAGCTCGCCCTGTGTT | 80 |
| PRSS23 | 202458_at | −0.34 | GTTCCCCACAGGATTTCAACGTGGC | 81 |
| PTRF | 208790_s_at | −0.338 | TTGCAGTGATTCCACGGTTAGGCCC | 82 |
| TJP1 | 202011_at | −0.337 | AAGCAGCGTTTCTATGACACATGCA | 83 |
| CRK | 202225_at | −0.336 | TCAGTGATCCAGGGCCGTTCATGAA | 84 |
| LASP1 | 200618_at | −0.334 | CTGGCCTGTTATGATTCTGAACATT | 85 |
| PRC1 | 218009_s_at | −0.334 | GTGTTCAGTTCTGTTACACAGTGCA | 86 |
| TMEM189 | 201003_x_at | −0.332 | GAAGTATACACTTCCGCTGTACCAC | 87 |
| TMEM189- | | | | |
| UBE2V1 | | | | |
| UBE2V1 | | | | |
| JAK1 | 201648_at | −0.325 | ATCCCTGTTTTTACCATCAATCATC | 88 |
| ACTN4 | 200601_at | −0.323 | GAGGAGCTGAGTTGGCAGACCGGGC | 89 |
| FAM45A | 221804_s_at | −0.323 | GCTGCCTGAAACGTTGCTTTGTATT | 90 |
| FAM45B | | | | |
| BIN1 | 214439_x_at | −0.319 | CGCAGACGGTCTGTGTGCTGTTTGA | 91 |
| PPP2CB | 201375_s_at | −0.316 | GGTCAGTCTAACAGTTTGCCTGCTG | 92 |
| EGFR | 201983_s_at | −0.316 | CATTAGCTCTTAGACCCACAGACTG | 93 |
| CNN3 | 201445_at | −0.315 | GAAAAATTGCCTTACGTACATTCCT | 94 |
| ARL6IP1 | 211935_at | −0.315 | GGCCTCTGGTCTACAAAGATTTCAT | 95 |
| FAIM | 220643_s_at | −0.315 | TGAAACTAACATTCCAAGGGTCAGG | 96 |
| CDC20 | 202870_s_at | −0.314 | AGTACCCAACCATGGCCAAGGTGGC | 97 |
| KIF23 | 204709_s_at | −0.314 | TGCAAGAATGCACTTTAGAACTATT | 98 |
| BIN1 | 210201_x_at | −0.314 | CGCAGACGGTCTGTGTGCTGTTTGA | 99 |
| SDC4 | 202071_at | −0.312 | CAGCGAGAGCATGTCCATCTGTTGG | 100 |
| EZR | 208623_s_at | −0.312 | TGGACTATGCACACTTTTAATTTTG | 101 |
| FGFR1 | 211535_s_at | −0.312 | CTTGTCCTCAGGGCTACAGCAGTAG | 102 |
| LIMA1 | 217892_s_at | −0.312 | ACTTGAGCTCAGACCTCTAAACCCT | 103 |
| ITGA3 | 201474_s_at | −0.31 | ATGGCGGGATCCTCCACAGAGAGGA | 104 |
| TUBB6 | 209191_at | −0.31 | CAACACGCAAGTTCCTTCTTGAACC | 105 |
| DYNLT1 | 201999_s_at | −0.308 | GGCACCGAAGTCAGATGAGTATCCC | 106 |
| KRT7 | 209016_s_at | −0.307 | GAGCTGGCGCTCAAGGATGCTCGTG | 107 |
| ANXA3 | 209369_at | −0.306 | AACAGCAACTTGTGTTCCTAACAGG | 108 |
| MAPK1 | 212271_at | −0.306 | GGAGCTCTATCCATATTTTACTGAT | 109 |
| DOCK9 | 212538_at | −0.306 | AGCTGTCACCATGTTATATTTTCTT | 110 |
| ZMYM6 | 213698_at | −0.306 | GGAAGTAGAGCATCTCTGTCTCTTT | 111 |
| ZMYM6NB | | | | |
| GFPT1 | 202721_s_at | −0.302 | GAGGCTATGATGTTGATTTCCCACG | 112 |
| MAP7D1 | 217943_s_at | −0.301 | TCTGCACCTTATAGACTGATGTCTC | 113 |
| BID | 211725_s_at | −0.299 | GCATGTCAACAGCGTTCCTAGAGAA | 114 |
| NMT1 | 201157_s_at | −0.296 | GCAGAACTGAACCGGCTTTACCAAA | 115 |
| TRAM1 | 201398_s_at | −0.296 | CAGTGGAACCAAATTTTTGCCATTA | 116 |
| GPRC5A | 212444_at | −0.296 | TTTAGCCCTCATGACTGTATTTTCT | 117 |
| MICALL1 | 55081_at | −0.295 | CCACCGAAGGCCTGATGGCTACTCA | 118 |
| WDR1 | 200611_s_at | −0.294 | ATGATGCCTCTGTCAAGGAGTGGAC | 119 |
| TRAF4 | 202871_at | −0.294 | TGACCTCAGTCAGGCACTGGCTGAA | 120 |
| AMOTL2 | 203002_at | −0.294 | CTGGTCTTAAAGAGTCCCTCACTTC | 121 |
| AGRN | 212285_s_at | −0.294 | GTATGTTTCTGTGTCAATCGCTGTG | 122 |
| OBSL1 | 212775_at | −0.294 | TCCGGAGTTATGCGTCCTCTTGAAA | 123 |
| LAPTM4B | 208029_s_at | −0.292 | ACATGGGGTGACATGCCTCGTATGT | 124 |
| MGAT4B | 220189_s_at | −0.291 | CGCAGTGGGAACATCGAGCACCCGG | 125 |
| IQGAP1 | 200791_s_at | −0.29 | GAGGATGCCCCAACAAACTCATGGC | 126 |
| CTBP2 | 201220_x_at | −0.29 | AAGTCTACAGGGGCTGGTGACCTCT | 127 |
| AKR1B1 | 201272_at | −0.29 | GCAGCCAGGATATGACCACCTTACT | 128 |
| CAPN2 | 208683_at | −0.29 | ATCCTGCGCTTGATCAACTGAACCA | 129 |
| TRAM1 | 201399_s_at | −0.289 | GTTAGAATCGCTGTTCTGGCATCCA | 130 |
| LACTB2 | 218701_at | −0.289 | TATTTTCTTATTCTGGTATACCACA | 131 |
| BIN1 | 202931_x_at | −0.288 | GCAGCCTCCGGGCGTGTGAAGAACA | 132 |
| PTPRK | 203038_at | −0.286 | CTCTTTCTAGATTGCCAGCTCATGA | 133 |
| CD24 | 208650_s_at | −0.286 | TACTAATTCCACACCTTTTATTGAC | 134 |
| CD24 | 209771_x_at | −0.286 | GCCTCGACACACATAAACCTTTTTA | 135 |
| PDLIM7 | 203370_s_at | −0.285 | GTGCCCTATTGCGAGCGAGACTATG | 136 |
| ZNF238 | 212774_at | −0.285 | AAACATAGCCTATTTTTGIGCTTAA | 137 |
| FAM129A | 217967_s_at | −0.285 | GATTCCAACTTGTCCAAAAGCTTTC | 138 |
| FAT1 | 201579_at | −0.284 | GGACCTTCTGCATACCTGTTTAGAA | 139 |
| PPP4R1 | 201594_s_at | −0.283 | AAGGACCTGATGCCAACACAAGTAG | 140 |
| TPX2 | 210052_s_at | −0.282 | GCCTTTTATATGGGTCTTCACTCTG | 141 |
| SOGA2 | 213358_at | −0.282 | GCTTGCACGTCTTCGGGTGCATGTA | 142 |
| FNTA | 200090_at | −0.281 | TCCCTTTGCCTGTGGTGTAAAAGTG | 143 |
| CD24 | 216379_x_at | −0.281 | GCCTCGACACACATAAACCTTTTTA | 144 |
| FNDC3B | 218618_s_at | −0.281 | TACAGGTTCCGCGTATGTGCGTGTC | 145 |
| SLC3A2 | 200924_s_at | −0.28 | GGCTCCAGTCATGCTGTGGGATGAG | 146 |
| S100A10 | 200872_at | −0.279 | CGCTCCTCCCTGATAAGAGTTGTCC | 147 |
| RPA1 | 201529_s_at | −0.279 | GGCCGAAGGCTGGTCATGAGCATCA | 148 |
| HOXB2 | 205453_at | −0.279 | TTCCGTTTGGTAGACTCCTTCCAAT | 149 |
| PI4KA PI4KAP1 | 213408_s_at | −0.279 | GCGAGGCTGCAAATTTCATCATGAA | 150 |
| PI4KAP2 | | | | |
| TRAF3 | 221571_at | −0.279 | AAGAAGGCCAATCTCTCATTTGACC | 151 |
| BHLHE40 | 201170_s_at | −0.274 | GATCCTTTCTGTAGGCTAATTCCTC | 152 |
| CAV2 | 203323_at | −0.273 | GATGGGACGCATAATCATTACCTTA | 153 |
| EPB41L2 | 201719_s_at | −0.271 | AAAGCTGGCACCAGAGACCCGATAG | 154 |
| HDGFRP3 | 209526_s_at | −0.271 | CCCACCATCATCAAACACTCAGTTA | 155 |
| AMIGO2 | 222108_at | −0.271 | AATAGACCCTACTGTACTGTGCTTT | 156 |
| CD24 | 266_s_at | −0.271 | TATTCCAGAATGCTGTACATCTATT | 157 |
| CARD10 | 210026_s_at | −0.27 | GGCGGCTGCTCTGTCATGAGAATGT | 158 |
| LAPTM4B | 214039_s_at | −0.27 | CCAAGTATGTCTAGTCACCTTTTAA | 159 |
| MICA | 205904_at | −0.269 | AGGCAGCTGGGATTCAATTCCCTGC | 160 |
| TNFRSF10B | 209295_at | −0.269 | TTTGCTATATCCCCAGGCCAAATAG | 161 |
| ZDHHC7 | 218606_at | −0.269 | GTCTGAGGGTCTTCCTTTATGCTTG | 162 |
| SLC25A1 | 210010_s_at | −0.268 | CTAGAGAGGCCGCAAGGGGACCGCC | 163 |
| ARPC5 | 211963_s_at | −0.268 | CATGGCCCCTCAATTTATTTGTGGT | 164 |
| NF1 | 212676_at | −0.268 | GTAGGGGGGCTGTTAGAATTGCTGC | 165 |
| GPRC5A | 203108_at | −0.267 | TCCCCAAACTTGCTGTCAATTCCGA | 166 |
| ACTB ACTB | AFFX- | −0.265 | TACCACTGGCATCGTGATGGACTCC | 167 |
| LOC100505829 | HSAC07/ | | | |
| | X00351_5_at | | | |
| KRT8 | 209008_x_at | −0.264 | GCGGCTATGCAGGTGGTCTGAGCTC | 168 |
| MKI67 | 212021_s_at | −0.264 | GACACTTGCCACACTGTGTCGTCGT | 169 |
| ANXA4 | 201302_at | −0.263 | GTTAATGTCTTAATCCAGCTTGCAA | 170 |
| COTL1 | 221059_s_at | −0.262 | CTTGGGCCGAAGGGTTGCTCTGCCC | 171 |
| LGALS3BP | 200923_at | −0.261 | TCACCAAGTCTGGCGGCTCAGATCG | 172 |
| PLOD2 | 202620_s_at | −0.26 | TTATCAAGTGTCAAGATCAGCAAGT | 173 |
| MPZL1 | 210087_s_at | −0.26 | GATTACACTGGCTGCAGTACATCAG | 174 |
| RND3 | 212724_at | −0.26 | GCAGCCAAGGTCTGTGTTCAGCACT | 175 |
| FZD6 | 203987_at | −0.259 | GTGTGATTTTTATAGTCTCGTTTTA | 176 |
| LIF | 205266_at | −0.257 | TACCCTGTGGGGATGTTTCATACTG | 177 |
| TNFRSF1A | 207643_s_at | −0.257 | GAACTGTCCTAAGGCAGGGGCGAGC | 178 |
| AURKA | 208079_s_at | −0.257 | AAATAGGAACACGTGCTCTACCTCC | 179 |
| NR2F2 | 209121_x_at | −0.257 | GTCGCCTTTATGGACCACATACGGA | 180 |
| COPB1 | 201358_s_at | −0.256 | AGTTCTGAATGCTGTCCTCAAAGTA | 181 |
| CD44 | 204490_s_at | −0.256 | GAATCAGATGGACACTCACATGGGA | 182 |
| SMAD3 | 205398_s_at | −0.256 | TCGATGAGCGCCACCTCTTTAAAAA | 183 |
| CLPTM1 | 211136_s_at | −0.256 | CCTGTTCGCCTTTGTCATCAAGATG | 184 |
| LPHN2 | 206953_s_at | −0.255 | CAAGCCACAGGCCTTATTTCATATG | 185 |
| NR2F2 | 209120_at | −0.255 | TAAGTGCAACATTTCTGTATACTGT | 186 |
| CAV2 | 203324_s_at | −0.254 | AAAGCACACAACGATTATAGTAACT | 187 |
| SGCE | 204688_at | −0.253 | AACATGCCATTGATGCAAACGCAGC | 188 |
| FAM134B | 218532_s_at | −0.253 | ACTGTGTTTGATGTCCTTTATTGAT | 189 |
| IL6 | 205207_at | −0.252 | GGGCACCTCAGATTGTTGTTGTTAA | 190 |
| RAB32 | 204214_s_at | −0.251 | ATCTTCCAAATGGCAGCCCTATCCC | 191 |
| FLNB | 208613_s_at | −0.251 | GTGGGCCAGAAGAGTTCCTTCCTGG | 192 |
| CD44 | 210916_s_at | −0.251 | GCTGACCTCTGCAAGGCTTTCAATA | 193 |

Note that RRAS2 is targeted by two different probesets (SEQ ID NOs: 49 and 50), PTRF is targeted by two different probesets (SEQ ID NOs: 55 and 82), ACTN1 is targeted by two different probesets (SEQ ID NOs: 59 and 61), NNMT is targeted by two different probesets (SEQ ID NOs: 64 and 66), BIN1 is targeted by three different probesets (SEQ ID NOs: 91, 99, and 132), GPRC5A is targeted by two different probesets (SEQ ID NOs: 117 and 166), LAPTM4B is targeted by two different probesets (SEQ ID NOs: 124 and 159), CD24 is targeted by four different probesets (SEQ ID NOs: 134, 135, 144, and 157), CAV2 is targeted by two different probesets (SEQ ID NOs: 153 and 187), NR2F2 is targeted by two different probesets (SEQ ID NOs: 180 and 186), and CD44 is targeted by two probesets (SEQ ID NOs: 182 and 193).

In particular, featured are methods for determining whether a patient may be responsive to ixabepilone or a pharmaceutically acceptable salt thereof by, e.g., detecting the expression level (e.g., mRNA or protein expression level) of one or more of the biomarkers shown in Table(s) 1 and/or 2 (e.g., HLA-DRA (SEQ ID NO: 1) and/or PLK2 (SEQ ID NO: 47)) in a biological sample (e.g., a tumor biopsy, such as, e.g., a breast cancer tumor biopsy) obtained from the subject using a device (e.g., a microarray). The expression level of one or more of the biomarkers of sensitivity may then be compared to the expression level of the biomarker(s) in a cell or tissue (e.g., a tumor tissue) known to be sensitive or resistant to ixabepilone or a pharmaceutically acceptable salt thereof (e.g., a tumor sample from a reference subject having the same diagnosis as the patient and that has been determined to be sensitive or resistant to ixabepilone or a pharmaceutically acceptable salt thereof) to determine the patient's responsiveness to ixabepilone or a pharmaceutically acceptable salt thereof.

For example, the patient may be deemed responsive to ixabepilone or a pharmaceutically acceptable salt thereof if the expression level of the one or more biomarkers of sensitivity (e.g., one or more of HLA-DRA (SEQ ID NOs: 1 and 2), ICAM3 (SEQ ID NO: 3), ITGB7 (SEQ ID NO: 4), CD8B (SEQ ID NO: 5), CD74 (SEQ ID NO: 6), HLA-DRB1 HLA-DRB4 HLA-DRB5 LOC100507709 LOC100507714 (SEQ ID NO: 7), IGJ (SEQ ID NO: 8), HLA-DRB1 HLA-DRB3 HLA-DRB4 LOC100507709 LOC100507714 (SEQ ID NO: 9), HLA-DPA1 (SEQ ID NOs: 10 and 12), HLA-DRB1 LOC100507709 LOC100507714 (SEQ ID NO: 11), CD28 (SEQ ID NO: 13), CD37 (SEQ ID NO: 14), NHP2 (SEQ ID NO: 15), MZB1 (SEQ ID NO: 16), ADCK3 (SEQ ID NO: 17), MYC (SEQ ID NO: 18), MEF2C (SEQ ID NOs: 19 and 24), RNASE6 (SEQ ID NO: 20), SRM (SEQ ID NO: 21), HAPLN1 (SEQ ID NO: 22), CYTIP (SEQ ID NO: 23), EIF3C EIF3CL (SEQ ID NO: 25), IQGAP2 (SEQ ID NO: 26), WBSCR22 (SEQ ID NO: 27), PIM2 (SEQ ID NO: 28), NCKAP1L (SEQ ID NO: 29), HCLS1 (SEQ ID NO: 30), MAGEA9 MAGEA9B (SEQ ID NO: 31), CTSS (SEQ ID NO: 32), GTF3A (SEQ ID NOs: 33 and 37), LCP1 (SEQ ID NO: 34), FAIM3 (SEQ ID NO: 35), HLA-DMB (SEQ ID NO: 36), RTP4 (SEQ ID NO: 38), MXI1 (SEQ ID NO: 39), SELPLG (SEQ ID NO: 40), IL10RA (SEQ ID NO: 41), POLR2H (SEQ ID NO: 42), BZW2 (SEQ ID NO: 43), CCDC88C (SEQ ID NO: 44), CORO1A (SEQ ID NO: 45), and AKAP1 (SEQ ID NO: 46)) is substantially similar to the expression level of the biomarker(s) of sensitivity in a cell (e.g., a cancer cell) or tissue (e.g., a tumor) known to be sensitive to ixabepilone or a pharmaceutically acceptable salt thereof (e.g., a tumor sample from a reference subject having the same diagnosis as the patient and that has been determined to be responsive to ixabepilone or a pharmaceutically acceptable salt thereof). For example, a patient may be deemed sensitive to treatment with ixabepilone or a pharmaceutically acceptable salt thereof if the expression level of the biomarker(s) of sensitivity in a sample is above a cutoff value of the 50^{th} percentile or greater (e.g., 60^{th} percentile, 70^{th} percentile, 80^{th} percentile, or 90^{th} percentile, or greater) in a cell or tissue (e.g., a tumor tissue) obtained from a reference subject in a reference population known to be responsive to ixabepilone and having the same diagnosis as the patient. The patient may also be deemed responsive to ixabepilone or a pharmaceutically acceptable salt thereof if the level of expression of one or more of the biomarkers of resistance (e.g., one or more of PLK2 (SEQ ID NO: 47), PLXNB2 (SEQ ID NO: 48), RRAS2 (SEQ ID NO: 49 and 50), PTPLA (SEQ ID NO: 51), P2RX5-TAX1BP3 TAX1BP3 (SEQ ID NO: 52), STAT3 (SEQ ID NO: 53), PPIC (SEQ ID NO: 54), PTRF (SEQ ID NO: 55), RCN1 (SEQ ID NO: 56), ZFP36L1 (SEQ ID NO: 57), GFPT1 (SEQ ID NO: 58 and 112), ACTN1 (SEQ ID NOs: 59 and 61), SEPT10 (SEQ ID NO: 60), COL4A1 (SEQ ID NO: 62), ERBB2IP (SEQ ID NO: 63), NNMT (SEQ ID NOs: 64 and 66), ADAM9 (SEQ ID NO: 65), TOR1AIP1 (SEQ ID NO: 67), ATP1B1 (SEQ ID NO: 68), CEBPD (SEQ ID NO: 69), FLII (SEQ ID NO: 70), FHL2 (SEQ ID NO: 71), SHC1 (SEQ ID NO: 72), SPTAN1 (SEQ ID NO: 73), CD81 (SEQ ID NO: 74), IQGAP1 (SEQ ID NO: 75), TGIF1 (SEQ ID NO: 76), PHACTR2 (SEQ ID NO: 77), COL4A2 (SEQ ID NO: 78), CEP55 (SEQ ID NO: 79), ABCC1 (SEQ ID NO: 80), PRSS23 (SEQ ID NO: 81), PTRF (SEQ ID NO: 82), TJP1 (SEQ ID NO: 83), CRK (SEQ ID NO: 84), LASP1 (SEQ ID NO: 85), PRC1 (SEQ ID NO: 86), TMEM189 TMEM189-UBE2V1 UBE2V1 (SEQ ID NO: 87), JAK1 (SEQ ID NO: 88), ACTN4 (SEQ ID NO: 89), FAM45A FAM45B (SEQ ID NO: 90), BIN1 (SEQ ID NOs: 91, 99, and 132), PPP2CB (SEQ ID NO: 92), EGFR (SEQ ID NO: 93), CNN3 (SEQ ID NO: 94), ARL6IP1 (SEQ ID NO: 95), FAIM (SEQ ID NO: 96), CDC20 (SEQ ID NO: 97), KIF23 (SEQ ID NO: 98), SDC4 (SEQ ID NO: 100), EZR (SEQ ID NO: 101), FGFR1 (SEQ ID NO: 102), LIMA1 (SEQ ID NO: 103), ITGA3 (SEQ ID NO: 104), TUBB6 (SEQ ID NO: 105), DYNLT1 (SEQ ID NO: 106), KRT7 (SEQ ID NO: 107), ANXA3 (SEQ ID NO: 108), MAPK1 (SEQ ID NO: 109), DOCK9 (SEQ ID NO: 110), ZMYM6 ZMYM6NB (SEQ ID NO: 111), MAP7D1 (SEQ ID NO: 113), BID (SEQ ID NO: 114), NMT1 (SEQ ID NO: 115), TRAM1 (SEQ ID NOs: 116 and 130), GPRC5A (SEQ ID NOs: 117 and 166), MICALL1 (SEQ ID NO: 118), WDR1 (SEQ ID NO: 119), TRAF4 (SEQ ID NO: 120), AMOTL2 (SEQ ID NO: 121), AGRN (SEQ ID NO: 122), OBSL1 (SEQ ID NO: 123), LAPTM4B (SEQ ID NOs: 124 and 159), MGAT4B (SEQ ID NO: 125), IQGAP1 (SEQ ID NO: 126), CTBP2 (SEQ ID NO: 127), AKR1B1 (SEQ ID NO: 128), CAPN2 (SEQ ID NO: 129), LACTB2 (SEQ ID NO: 131), PTPRK (SEQ ID NO: 133), CD24 (SEQ ID NOs: 134, 135, 144, and 157), PDLIM7 (SEQ ID NO: 136), ZNF238 (SEQ ID NO: 137), FAM129A (SEQ ID NO: 138), FAT1 (SEQ ID NO: 139), PPP4R1 (SEQ ID NO: 140), TPX2 (SEQ ID NO: 141), SOGA2 (SEQ ID NO: 142), FNTA (SEQ ID NO: 143), FNDC3B (SEQ ID NO: 145), SLC3A2 (SEQ ID NO: 146), S100A10 (SEQ ID NO: 147), RPA1 (SEQ ID NO: 148), HOXB2 (SEQ ID NO: 149), PI4KA P14KAP1 PI4KAP2 (SEQ ID NO: 150), TRAF3 (SEQ ID NO: 151), BHLHE40 (SEQ ID NO: 152), CAV2 (SEQ ID NOs: 153 and 187), EPB41L2 (SEQ ID NO: 154), HDGFRP3 (SEQ ID NO: 155), AMIGO2 (SEQ ID NO: 156), CARD10 (SEQ ID NO: 158), LAPTM4B (SEQ ID NO: 159), MICA (SEQ ID NO: 160), TNFRSF10B (SEQ ID NO: 161), ZDHHC7 (SEQ ID NO: 162), SLC25A1 (SEQ ID NO: 163), ARPC5 (SEQ ID NO: 164), NF1 (SEQ ID NO: 165), GPRC5A (SEQ ID NO: 166), ACTB ACTB LOC100505829 (SEQ ID NO: 167), KRT8 (SEQ ID NO: 168), MKI67 (SEQ ID NO: 169), ANXA4 (SEQ ID NO: 170), COTL1 (SEQ ID NO: 171), LGALS3BP (SEQ ID NO: 172), PLOD2 (SEQ ID NO: 173), MPZL1 (SEQ ID NO: 174), RND3 (SEQ ID NO: 175), FZD6 (SEQ ID NO: 176), LIF (SEQ ID NO: 177), TNFRSF1A (SEQ ID NO: 178), AURKA (SEQ ID NO: 179), NR2F2 (SEQ ID NOs: 180 and 186), COPB1 (SEQ ID NO: 181), CD44 (SEQ ID NOs: 182 and 193), SMAD3 (SEQ ID NO: 183), CLPTM1 (SEQ ID NO: 184), LPHN2 (SEQ ID NO: 185), SGCE (SEQ ID NO: 188), FAM134B (SEQ ID NO: 189), IL6 (SEQ ID NO: 190), RAB32 (SEQ ID NO: 191), and FLNB (SEQ ID NO: 192)) is substantially similar to the expression level of the biomarker(s) of resistance in a cell or tissue (e.g., a tumor tissue) known to be sensitive to ixabepilone or a pharmaceutically acceptable salt thereof (e.g., a tumor sample from a reference subject having the same diagnosis as the patient and that has been determined to be responsive to ixabepilone or a pharmaceutically acceptable salt thereof). The patient may also be deemed responsive to ixabepilone or a pharmaceutically acceptable salt thereof if the level of expression of one or more of the biomarkers of sensitivity is substantially dissimilar to the expression level of the biomarker(s) of sensitivity in a cell (e.g., a cancer cell) or tissue (e.g., a tumor) known to be resistant to ixabepilone or a pharmaceutically acceptable salt thereof (e.g., a tumor sample from a reference subject having the same diagnosis as the patient and that has been determined to be resistant to ixabepilone or a pharmaceutically acceptable salt thereof). Also, the patient may be deemed responsive to ixabepilone or a pharmaceutically acceptable salt thereof if the level of expression of one or more of the biomarkers of resistance is substantially dissimilar to the expression level of the biomarker(s) of resistance in a cell or tissue (e.g., a tumor tissue) known to be resistant to ixabepilone or a pharmaceutically acceptable salt thereof (e.g., a tumor sample from a reference subject having the same diagnosis as the patient and that has been determined to be resistant to ixabepilone or a pharmaceutically acceptable salt thereof). For example, a patient may be deemed sensitive to treatment with ixabepilone or a pharmaceutically acceptable salt thereof if the expression level of the biomarker(s) of resistance in a cell is below a cutoff value of the 50^{th} percentile or less (e.g., 40^{th} percentile, 30^{th} percentile, 20^{th} percentile, or 100^{th} percentile, or less) in a cell or tissue (e.g., a tumor tissue) obtained from a reference subject in a reference population known to be resistant to ixabepilone and having the same diagnosis as the patient,

Also featured are methods of treating a patient with a cancer (e.g., a patient diagnosed as having breast cancer), such as a patient having recurrence of cancer, by detecting the expression levels of one or more of the biomarkers shown in Tables 1 and/or 2 (e.g., HLA-DRA (SEQ ID NO: 1) or PLK2 (SEQ ID NO: 47)) in a sample (e.g., a tumor sample) from the patient, and then administering ixabepilone or a pharmaceutically acceptable salt thereof based on the expression levels of the biomarker(s). In particular, a patient with a cancer may be administered ixabepilone or a pharmaceutically acceptable salt thereof if the expression level of one or more biomarkers of sensitivity is substantially similar to the expression level of the biomarker(s) of sensitivity in a cell or tissue (e.g., a tumor tissue) known to be sensitive to ixabepilone or a pharmaceutically acceptable salt thereof. Moreover, a patient with a cancer may be administered ixabepilone or a pharmaceutically acceptable salt thereof if the expression level of one or more biomarkers of resistance is substantially similar to the expression level of the biomarker(s) of resistance in a cell or tissue (e.g., a tumor tissue) known to be sensitive to ixabepilone or a pharmaceutically acceptable salt thereof. Additionally, a patient with a cancer may be administered ixabepilone or a pharmaceutically acceptable salt thereof if the expression level of one or more biomarkers of sensitivity is substantially dissimilar to the expression level of the biomarker(s) of sensitivity in a cell or tissue (e.g., a tumor tissue) known to be resistant to ixabepilone or a pharmaceutically acceptable salt thereof. Also, a patient with a cancer may be administered ixabepilone or a pharmaceutically acceptable salt thereof if the expression level of one or more biomarkers of resistance is substantially dissimilar to the expression level of the biomarker(s) of resistance in a cell or tissue (e.g., a tumor tissue) known to be resistant to ixabepilone or a pharmaceutically acceptable salt thereof. Thus, the methods can be used to treat a cancer patient predicted to be responsive to ixabepilone or a pharmaceutically acceptable salt thereof, such as a patient with, e.g., breast cancer, estrogen receptor-positive (ERpos) breast cancer, metastatic breast cancer, multiple myeloma, acute myelogenous leukemia (AML), acute lympho-blastic leukemia (ALL), chronic lymphocytic leukemia (CLL), myelodysplastic syndrome (MDS), chronic myelogenous leukemia-chronic phase (CMLCP), diffuse large B-cell lymphoma (DLBCL), cutaneous T-cell lymphoma (CTCL), peripheral T-cell lymphoma (PTCL), Hodgkin's lymphoma, hepatocellular carcinoma (HCC), cervical cancer, prostate cancer, kidney cancer, renal cell carcinoma (RCC), esophageal cancer, melanoma, glioma, pancreatic cancer, ovarian cancer, gastrointestinal stromal tumors (GIST), sarcoma, endometrial cancer, lung cancer, non-small cell lung carcinoma (NSCLC), mesothelioma, intestinal cancer, colon cancer, bladder cancer, adrenal cancer, gallbladder cancer, or squamous cell carcinoma of the head and neck (SCCHN). In preferred embodiments, the method is used to treat a breast cancer patient predicted to be responsive to ixabepilone or a pharmaceutically acceptable salt thereof.

Methods are described herein for identifying biomarkers of drug responsiveness, detecting biomarker gene expression in a cancer patient, determining the responsiveness of a cancer patient to ixabepilone or a pharmaceutically acceptable salt thereof, and treating cancer in a patient with ixabepilone or a pharmaceutically acceptable salt thereof. Also described are devices and kits for use in these methods.

### Cancer Types

The methods, devices, and kits of the invention can be used for diagnosing, prognosing, monitoring, treating, and/or reducing cancer in a subject suffering from, diagnosed with, or susceptible to cancer. Non-limiting examples of cancers that can be diagnosed, prognosed, monitored, treated, or reduced using the methods include hematological and solid tumors. In particular, cancers include, e.g., breast cancer (e.g., medullary carcinoma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myeloma (e.g., multiple myeloma), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, prostate cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), ovarian cancer (e.g., ovarian adenocarcinoma or embryonal carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), ER-positive cancer, bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), metastatic cancer, oral cavity cancer, uterine cancer, testicular cancer (e.g., seminoma and embryonal carcinoma), skin cancer (e.g., squamous cell carcinoma and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, liver cancer (e.g., hepatocellular carcinoma or hepatoma), larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, and neoplasms of the central nervous system.

In particular, the methods are useful for diagnosing, prognosing, monitoring, treating, or preventing, e.g., breast cancer, multiple myeloma, acute myelogenous leukemia (AML), acute lympho-blastic leukemia (ALL), chronic lymphocytic leukemia (CLL), myelodysplastic syndrome (MDS), chronic myelogenous leukemia-chronic phase (CMLCP), diffuse large B-cell lymphoma (DLBCL), cutaneous T-cell lymphoma (CTCL), peripheral T-cell lymphoma (PTCL), Hodgkin's lymphoma, hepatocellular carcinoma (HCC), cervical cancer, prostate cancer, kidney cancer, renal cell carcinoma (RCC), esophageal cancer, melanoma, glioma, pancreatic cancer, ovarian cancer, gastrointestinal stromal tumors (GIST), sarcoma, estrogen receptor-positive (ERpos) breast cancer, metastatic breast cancer, endometrial cancer, lung cancer, non-small cell lung carcinoma (NSCLC), mesothelioma, intestinal cancer, colon cancer, bladder cancer, adrenal cancer, gallbladder cancer, and/or squamous cell carcinoma of the head and neck (SCCHN). For example, the cancer can be a breast cancer, such as medullary carcinoma. The cancer can be estrogen receptor-positive (ER pos) breast cancer. The cancer can be a metastatic form of breast cancer. The breast cancer can be, for example, a Stage 0, Stage I, Stage II, Stage III, or Stage IV breast cancer.

### Methods for Detecting Biomarker Gene Expression in a Cancer Patient

A cancer patient can be assessed for sensitivity or resistance to ixabepilone or a pharmaceutically acceptable salt thereof by detecting gene expression of a biomarker (e.g., one or more of the biomarkers of Tables 1 and/or 2) in a biological sample obtained from the cancer patient (e.g., a patient with the cancer, such as, e.g., breast cancer, or a recurrence thereof). The biological sample can include, for example, cells, tissue (e.g., a tissue sample obtained by biopsy), blood, serum, plasma, urine, sputum, cerebrospinal fluid, lymph tissue or fluid, or pancreatic fluid. For example, the biological sample can be fresh frozen or formalin-fixed paraffin embedded (FFPE) tissue obtained from the subject, such as a tumor sample (e.g., a biopsy) from the tissue of interest (e.g., breast, lymph nodes, thymus, spleen, bone marrow, colorectal, pancreatic, cervical, prostate, bladder, lung, gastrointestinal, head, neck, or ovarian tissue).

### RNA Extraction and Biomarker Expression Measurement

Cell samples or tissue samples may be snap frozen in liquid nitrogen until processing or fixed in formalin and embedded in paraffin. RNA may be extracted using, e.g., Trizol Reagent from Invitrogen following manufacturer's instructions, and detected directly or converted to cDNA for detection. RNA may be amplified using, e.g., MessageAmp kit from Ambion following manufacturer's instructions. Amplified RNA may be quantified using, e.g., HG-U133A or HG-U133_Plus2 GeneChip from Affymetrix Inc. and compatible apparatus e.g. GCS3000Dx from Affymetrix, using the manufacturer's instructions. The Affymetrix array typically contains 11 probes (also known as a probe set) specific to each gene. In general, confidence in a prediction of responsiveness or non-responsiveness increases with an increase in the number of probes used in the analysis. In Tables 1 and 2, a representative probe of the typical 11 probes is shown for each gene. The resulting biomarker expression measurements may be further analyzed as described herein. The procedures described can be implemented using, e.g., R software available from R-Project and supplemented with packages available from Bioconductor.

One or more of the biomarkers shown in Tables 1 and/or 2 (e.g., HLA-DRA (SEQ ID NO: 1) or PLK2 (SEQ ID NO: 47)) may be measured in a biological sample (e.g., a tumor sample) obtained from the cancer patient (e.g., a patient with any of the cancer types described herein, such as a patient with recurrence of cancer) using, e.g., polymerase chain reaction (PCR), reverse transcriptase PCR (RT-PCR), quantitative real-time PCR (qPCR), an array (e.g., a microarray), a genechip, pyrosequencing, nanopore sequencing, sequencing by synthesis, sequencing by expansion, single molecule real time technology, sequencing by ligation, microfluidics, infrared fluorescence, next generation sequencing (e.g., RNA-Seq techniques), Northern blots, Western blots, Southern blots, NanoString nCounter technologies (e.g., those described in U.S. Patent Application Nos. US 2011/0201515, US 2011/0229888, and US 2013/0017971, each of which is incorporated by reference in its entirety), proteomic techniques (e.g., mass spectrometry or protein arrays), and combinations thereof.

### Devices

Devices of the invention can be used for detecting the level of expression of one or more biomarkers shown in Table(s) 1 and/or 2. The device may include at least one single-stranded nucleic acid (e.g., a probe) having at least 85% sequence identity (e.g., 85%, 90%, 95%, 97%, 98%, 99%, or 100% sequence identity) to a nucleic acid sequence that is complementary or identical to at least 5 (e.g., at least 10, at least 15, at least 20, or more) consecutive nucleotides of one or more biomarkers shown in Table(s) 1 and/or 2 (e.g., HLA-DRA (SEQ ID NO: 1) or PLK2 (SEQ ID NO: 47)), in which the at least one single-stranded nucleic acid is sufficient for the detection of the level of expression of the one or more biomarkers. The device may be used to detect the expression level of a given biomarker by specific hybridization between the single-stranded nucleic acid and the biomarker (e.g., an mRNA, genomic DNA, or non-coding RNA), a nucleic acid encoding the biomarker (e.g., an mRNA), or a complementary nucleic acid thereof. The device may be, or may include, a microarray. The device may also include or be used with reagents and materials for next generation sequencing (e.g., sequencing by synthesis). The device may also include or be used with NanoString reagents and at least one nCounter cartridge. The device may be, or include a protein array, which contains one or more protein binding moieties (e.g., proteins, antibodies, nucleic acids, aptamers, affibodies, lipids, phospholipids, small molecules, labeled variants of any of the above, and any other moieties useful for protein detection as well known in the art) capable of detectably binding to the polypeptide product(s) of one or more biomarkers shown in Table(s) 1 and/or 2. For example, the device may have single-stranded nucleic acid molecule(s) having the sequence of or complementary to each of the biomarkers of sensitivity selected from the biomarkers of Table 1 and/or for each of the biomarkers of resistance selected from the biomarkers of Table 2 that are affixed to the device and can be used to detect the level of expression of the biomarkers, e.g., by hybridization.

### Microarrays

The level of expression of the biomarkers (e.g., the biomarkers listed in Table(s) 1 and/or 2 (e.g., HLA-DRA (SEQ ID NO: 1) or PLK2 (SEQ ID NO: 47)) may be determined using high-throughput expression profiling platforms, such as microarrays. In particular, a microarray for use in the methods for assessing the responsiveness of a subject with cancer (e.g., a patient with recurrence of cancer) to ixabepilone or a pharmaceutically acceptable salt thereof contains or is produced by generating oligonucleotide probes (e.g., DNA, cDNA, or RNA probes) capable of hybridizing to one or more biomarkers of interest (e.g., one or more of the biomarkers of Table(s) 1 and/or 2) or the complement sequences thereof. Each probe can have, e.g., at least 10, 15, 20, 25, 30, or more contiguous nucleic acid residues (e.g., at least 15) that are complementary or identical to a nucleic acid sequence of a selected biomarker. The probe nucleic acid sequence can also have at least 85% (e.g., 90%, 95%, 99%, or 100%) sequence identity to the nucleic acid sequence of the gene coding the biomarker (e.g., HLA-DRA (SEQ ID NO: 1) or PLK2 (SEQ ID NO: 47)) or the complement sequence thereof. In particular, the probe sequences can be complementary to all or a portion of the nucleic acid sequence of the biomarker(s).

For example, microarrays of the invention for determining ixabepilone (e.g., ixabepilone or a pharmaceutically acceptable salt thereof) responsiveness can include probes for one or more (e.g., at least 1, 5, 10, 15, or 20 (e.g., at least the top 1, 5, 10, 15, or 20), or more (e.g., all)) biomarkers of sensitivity shown in Table 1, such as HLA-DRA (SEQ ID NOs: 1 and 2), ICAM3 (SEQ ID NO: 3), ITGB7 (SEQ ID NO: 4), CD8B (SEQ ID NO: 5), CD74 (SEQ ID NO: 6), HLA-DRB1 HLA-DRB4 HLA-DRB5 LOC100507709 LOC100507714 (SEQ ID NO: 7), IGJ (SEQ ID NO: 8), HLA-DRB1 HLA-DRB3 HLA-DRB4 LOC100507709 LOC100507714 (SEQ ID NO: 9), HLA-DPA1 (SEQ ID NOs: 10 and 12), HLA-DRB1 LOC100507709 LOC100507714 (SEQ ID NO: 11), CD28 (SEQ ID NO: 13), CD37 (SEQ ID NO: 14), NHP2 (SEQ ID NO: 15), MZB1 (SEQ ID NO: 16), ADCK3 (SEQ ID NO: 17), MYC (SEQ ID NO: 18), MEF2C (SEQ ID NOs: 19 and 24), RNASE6 (SEQ ID NO: 20), SRM (SEQ ID NO: 21), HAPLN1 (SEQ ID NO: 22), CYTIP (SEQ ID NO: 23), EIF3C EIF3CL (SEQ ID NO: 25), IQGAP2 (SEQ ID NO: 26), WBSCR22 (SEQ ID NO: 27), PIM2 (SEQ ID NO: 28), NCKAP1L (SEQ ID NO: 29), HCLS1 (SEQ ID NO: 30), MAGEA9 MAGEA9B (SEQ ID NO: 31), CTSS (SEQ ID NO: 32), GTF3A (SEQ ID NOs: 33 and 37), LCP1 (SEQ ID NO: 34), FAIM3 (SEQ ID NO: 35), HLA-DMB (SEQ ID NO: 36), RTP4 (SEQ ID NO: 38), MXI1 (SEQ ID NO: 39), SELPLG (SEQ ID NO: 40), MORA (SEQ ID NO: 41), POLR2H (SEQ ID NO: 42), BZW2 (SEQ ID NO: 43), CCDC88C (SEQ ID NO: 44), CORO1A (SEQ ID NO: 45), and AKAP1 (SEQ ID NO: 46).

Microarrays of the invention for determining ixabepilone (e.g., ixabepilone or a pharmaceutically acceptable salt thereof) responsiveness can also include probes for one or more (e.g., at least 1, 5, 10, 15, or 20 (e.g., at least the top 1, 5, 10, 15, or 20), or more (e.g., all)) biomarkers of resistance listed in Table 2, such as PLK2 (SEQ ID NO: 47), PLXNB2 (SEQ ID NO: 48), RRAS2 (SEQ ID NO: 49 and 50), PTPLA (SEQ ID NO: 51), P2RX5-TAX1BP3 TAX1BP3 (SEQ ID NO: 52), STAT3 (SEQ ID NO: 53), PPIC (SEQ ID NO: 54), PTRF (SEQ ID NO: 55), RCN1 (SEQ ID NO: 56), ZFP36L1 (SEQ ID NO: 57), GFPT1 (SEQ ID NO: 58 AND 112), ACTN1 (SEQ ID NOs: 59 and 61), SEPT10 (SEQ ID NO: 60), COL4A1 (SEQ ID NO: 62), ERBB2IP (SEQ ID NO: 63), NNMT (SEQ ID NOs: 64 and 66), ADAM9 (SEQ ID NO: 65), TOR1AIP1 (SEQ ID NO: 67), ATP1B1 (SEQ ID NO: 68), CEBPD (SEQ ID NO: 69), FLII (SEQ ID NO: 70), FHL2 (SEQ ID NO: 71), SHC1 (SEQ ID NO: 72), SPTAN1 (SEQ ID NO: 73), CD81 (SEQ ID NO: 74), IQGAP1 (SEQ ID NO: 75), TGIF1 (SEQ ID NO: 76), PHACTR2 (SEQ ID NO: 77), COL4A2 (SEQ ID NO: 78), CEP55 (SEQ ID NO: 79), ABCC1 (SEQ ID NO: 80), PRSS23 (SEQ ID NO: 81), PTRF (SEQ ID NO: 82), TJP1 (SEQ ID NO: 83), CRK (SEQ ID NO: 84), LASP1 (SEQ ID NO: 85), PRC1 (SEQ ID NO: 86), TMEM189 TMEM189-UBE2V1 UBE2V1 (SEQ ID NO: 87), JAK1 (SEQ ID NO: 88), ACTN4 (SEQ ID NO: 89), FAM45A FAM45B (SEQ ID NO: 90), BIN1 (SEQ ID NOs: 91, 99, and 132), PPP2CB (SEQ ID NO: 92), EGFR (SEQ ID NO: 93), CNN3 (SEQ ID NO: 94), ARL6IP1 (SEQ ID NO: 95), FAIM (SEQ ID NO: 96), CDC20 (SEQ ID NO: 97), KIF23 (SEQ ID NO: 98), SDC4 (SEQ ID NO: 100), EZR (SEQ ID NO: 101), FGFR1 (SEQ ID NO: 102), LIMA1 (SEQ ID NO: 103), ITGA3 (SEQ ID NO: 104), TUBB6 (SEQ ID NO: 105), DYNLT1 (SEQ ID NO: 106), KRT7 (SEQ ID NO: 107), ANXA3 (SEQ ID NO: 108), MAPK1 (SEQ ID NO: 109), DOCK9 (SEQ ID NO: 110), ZMYM6 ZMYM6NB (SEQ ID NO: 111), MAP7D1 (SEQ ID NO: 113), BID (SEQ ID NO: 114), NMT1 (SEQ ID NO: 115), TRAM1 (SEQ ID NOs: 116 and 130), GPRC5A (SEQ ID NOS: 117 AND 166), MICALL1 (SEQ ID NO: 118), WDR1 (SEQ ID NO: 119), TRAF4 (SEQ ID NO: 120), AMOTL2 (SEQ ID NO: 121), AGRN (SEQ ID NO: 122), OBSL1 (SEQ ID NO: 123), LAPTM4B (SEQ ID NOS: 124 AND 159), MGAT4B (SEQ ID NO: 125), IQGAP1 (SEQ ID NO: 126), CTBP2 (SEQ ID NO: 127), AKR1B1 (SEQ ID NO: 128), CAPN2 (SEQ ID NO: 129), LACTB2 (SEQ ID NO: 131), PTPRK (SEQ ID NO: 133), CD24 (SEQ ID NOs: 134, 135, 144, and 157), PDLIM7 (SEQ ID NO: 136), ZNF238 (SEQ ID NO: 137), FAM129A (SEQ ID NO: 138), FAT1 (SEQ ID NO: 139), PPP4R1 (SEQ ID NO: 140), TPX2 (SEQ ID NO: 141), SOGA2 (SEQ ID NO: 142), FNTA (SEQ ID NO: 143), FNDC3B (SEQ ID NO: 145), SLC3A2 (SEQ ID NO: 146), S100A10 (SEQ ID NO: 147), RPA1 (SEQ ID NO: 148), HOXB2 (SEQ ID NO: 149), PI4KA P14KAP1 PI4KAP2 (SEQ ID NO: 150), TRAF3 (SEQ ID NO: 151), BHLHE40 (SEQ ID NO: 152), CAV2 (SEQ ID NOs: 153 and 187), EPB41L2 (SEQ ID NO: 154), HDGFRP3 (SEQ ID NO: 155), AMIGO2 (SEQ ID NO: 156), CARD10 (SEQ ID NO: 158), LAPTM4B (SEQ ID NO: 159), MICA (SEQ ID NO: 160), TNFRSF10B (SEQ ID NO: 161), ZDHHC7 (SEQ ID NO: 162), SLC25A1 (SEQ ID NO: 163), ARPC5 (SEQ ID NO: 164), NF1 (SEQ ID NO: 165), GPRC5A (SEQ ID NO: 166), ACTB ACTB LOC100505829 (SEQ ID NO: 167), KRT8 (SEQ ID NO: 168), MKI67 (SEQ ID NO: 169), ANXA4 (SEQ ID NO: 170), COTL1 (SEQ ID NO: 171), LGALS3BP (SEQ ID NO: 172), PLOD2 (SEQ ID NO: 173), MPZL1 (SEQ ID NO: 174), RND3 (SEQ ID NO: 175), FZD6 (SEQ ID NO: 176), LIF (SEQ ID NO: 177), TNFRSF1A (SEQ ID NO: 178), AURKA (SEQ ID NO: 179), NR2F2 (SEQ ID NOs: 180 and 186), COPB1 (SEQ ID NO: 181), CD44 (SEQ ID NOs: 182 and 193), SMAD3 (SEQ ID NO: 183), CLPTM1 (SEQ ID NO: 184), LPHN2 (SEQ ID NO: 185), SGCE (SEQ ID NO: 188), FAM134B (SEQ ID NO: 189), IL6 (SEQ ID NO: 190), RAB32 (SEQ ID NO: 191), and FLNB (SEQ ID NO: 192).

A microarray probe may be single-stranded or double-stranded. The probe may be labeled (e.g., detectably labeled with a fluorescent molecule, dye molecule, small molecule, epitope tag, barcode sequence, polypeptide, or any other detectable molecule). Probes can be detectably labeled and immobilized on a solid support to form the microarray. For example, probes can be either prefabricated and spotted to the surface or directly synthesized on to the surface (in situ) of the microarray. The microarray can also be configured such that the sequence and position of each member (e.g., probe) of the array is known. For example, a selection of biomarkers whose expression correlates with an increased likelihood of responsiveness to ixabepilone or a pharmaceutically acceptable salt thereof can be arrayed on a solid support. Hybridization of a labeled probe with a particular target nucleic acid (e.g., an mRNA corresponding to one or more biomarkers of Table(s) 1 and/or 2) indicates that the sample from which the mRNA was derived expresses that biomarker (e.g., the biomarker of sensitivity or resistance to ixabepilone or a pharmaceutically acceptable salt thereof).

### PCR-Based Techniques

As few as one and up to 25 or more of the biomarkers (e.g., 1 to 25 (e.g., the top 1 to 25) or 10 to 25 (e.g., the top 10 to 25), or at least the top 25 of the biomarkers listed in Table(s) 1 and/or 2) may be used to determine responsiveness of a cancer patient to ixabepilone or a pharmaceutically acceptable salt thereof using the methods described herein. Tissue or cell samples from a cancer patient (e.g., a patient having recurrence of cancer, such as, e.g., breast cancer) can be conveniently assayed for gene expression levels using nucleic acid amplification methods, such as polymerase chain reaction (PCR). Such PCR-based techniques may include reverse transcription PCR (RT-PCR), quantitative real-time PCR (qPCR), reverse transcription qPCR (RT-qPCR), or quantitative loop-mediated isothermal amplification (q-LAMP). For example, an mRNA corresponding to a biomarker of Table 1 or 2 can be detected in a biological sample by (a) producing cDNA from the sample by reverse transcription using at least one primer; (b) amplifying the cDNA so produced using a target polynucleotide as sense and antisense primers to amplify target cDNAs therein; and (c) detecting the presence of the amplified target cDNA using polynucleotide probes. The primers and probes including the target sequences shown in Table(s) 1 and/or 2, such as HLA-DRA (SEQ ID NO: 1) or PLK2 (SEQ ID NO: 47), may be used to detect the level of expression of one or more of the indicated biomarkers using PCR. The methods can include one or more steps that allow determination of the levels of target mRNA in a biological sample (e.g., by simultaneously examining the levels of a comparative control mRNA sequence or “housekeeping” gene, such as an actin family member or GAPDH). The primers for these PCR-based techniques may be labeled for detection according to methods known in the art.

### Sequencing

The level of expression of the biomarkers shown in Table(s) 1 and/or 2, such as HLA-DRA (SEQ ID NO: 1) or PLK2 (SEQ ID NO: 47), may be determined using sequencing technologies, such as next generation sequencing platforms (e.g., RNA-Seq), as described in Mortazavi et al., Nat. Methods 5: 621-628, 2008, hereby incorporated by reference. RNA-Seq is a robust technology for monitoring expression by direct sequencing of the RNA molecules in a sample. This methodology may include fragmentation of RNA to an average length of, e.g., 200 nucleotides, conversion to cDNA by random priming, and synthesis of double-stranded cDNA (e.g., using the Just cDNA DoubleStranded cDNA Synthesis Kit from Agilent Technology). The cDNA may then be converted into a molecular library for sequencing by addition of sequence adapters for each library (e.g., from Illumina®/Solexa), and the resulting 50 to 100 nucleotide reads are mapped onto the genome. Exemplary sequencing platforms suitable for use according to the methods include, e.g., 454 pyrosequencing, Illumina sequencing by synthesis, SOLiD sequencing, Ion Torrent sequencing, and PacBio RS sequencing.

### Methods of Determining the Responsiveness of a Patient to Ixabepilone

The invention features diagnostic methods for the detection and screening of cancer patients (e.g., patients with cancer or a recurrence thereof) that may be responsive to ixabepilone or a pharmaceutically acceptable salt thereof using one or more of the biomarkers shown in Table(s) 1 and/or 2 (e.g., HLA-DRA (SEQ ID NO: 1) or PLK2 (SEQ ID NO: 47)). The methods of the invention may be used for predicting a patient's responsiveness to ixabepilone or a pharmaceutically acceptable salt thereof, and optionally, treating the cancer patient throughout the progression of cancer and/or in cases of recurrence (e.g., after a first line treatment, a second line treatment, and/or a third line treatment).

The invention provides individual biomarkers (e.g., HLA-DRA (SEQ ID NO: 1) or PLK2 (SEQ ID NO: 47)) and sets of biomarkers (e.g., two or more of the biomarkers listed in Table(s) 1 and/or 2), the expression levels of which, as detected in a biological sample (e.g., a tumor sample, such as a biopsy) obtained from a cancer patient (e.g., a human with cancer), are indicative of responsiveness to ixabepilone or a pharmaceutically acceptable salt thereof. The biomarkers were identified using methods similar to those previously described in, e.g., Chen et al. (Mol. Cancer Ther. 11:34-33, 2012), Wang et al. (J. Nat. Cancer Inst. 105: 1284-1291, 2013), and Knudsen et al. (PLoS One, 9: e87415, 2014), Buhl et al (PLoS One 13(3):e0194609, 2018) each of which are incorporated by reference herein in their entirety. In particular, an algorithm based on growth inhibition values (GI50) of a cell line (e.g., NCI60 cells) is used. The cell line is subjected to treatment with ixabepilone or a pharmaceutically acceptable salt thereof and baseline gene expression is determined (e.g., by microarray analysis, RT-PCR, qPCR, or next generation sequencing). After normalization, genes with, e.g., a Pearson correlation coefficient greater than 0.25 or below −0.25 can be classified as biomarkers of sensitivity or resistance, respectively. In particular, a correlation coefficient of 0.25 or greater is a statistically significant cut-off known in the art for establishing whether the expression levels of, e.g., the genes shown in Table(s) 1 and/or 2, correlate with the likelihood of cancer treatment sensitivity, such as sensitivity to ixabepilone or a pharmaceutically acceptable salt thereof, as described in van't Veer et al. Nature 415(6871):530-536, 2002, hereby incorporated by reference.

Alternatively, after normalization, genes, or means of genes, or differences between means of genes (e.g., the difference between the mean expression level of one or more biomarkers of sensitivity and the mean expression level of one or more biomarkers of resistance), that have an expression level above a cutoff value of the 50^{th} percentile or greater (e.g., 60^{th} percentile, 70^{th} percentile, 80^{th} percentile, or 90^{th} percentile, or greater) in a reference population with the same diagnosis as the patient (e.g., the same type of cancer) indicates that the sample, such as, e.g., a tumor sample (or the subject from whom the sample was taken), is predicted to be responsive (e.g., based on the biomarkers of sensitivity) or non-responsive (e.g., based on the biomarkers of resistance), respectively, to the treatment, e.g., treatment with ixabepilone or a pharmaceutically acceptable salt thereof.

### Comparison of Biomarker Expression Levels

One or more biomarkers of sensitivity and/or resistance, identified as described herein, can be used to predict responsiveness to ixabepilone or a pharmaceutically acceptable salt thereof by measuring the level of expression of the biomarker(s) in a biological sample obtained from the cancer patient. A single biomarker (e.g., any of the biomarkers of Table 1 or 2, such as HLA-DRA (SEQ ID NO: 1) or PLK2 (SEQ ID NO: 47)) or a set of biomarkers (e.g., one, two, three, four, five, ten, fifteen, twenty, twenty five, or all of the biomarkers of Tables 1 and/or 2 (e.g., the top one, the top two, the top three, the top four, the top five, the top ten, the top fifteen, the top twenty, the top twenty five, or all of the biomarkers of Tables 1 and/or 2)) may be used to determine the responsiveness of a cancer patient (e.g., a patient with cancer recurrence) to ixabepilone or a pharmaceutically acceptable salt thereof. After determining the level of expression of the biomarker(s) in a sample (e.g., a tumor sample) from the cancer patient, the level of expression of the biomarker(s) in the sample may be compared to the level of expression of the biomarker(s) in a cell (e.g., a cancer cell) or tissue (e.g., a tumor tissue, such as the same type of tumor as that of the cancer patient) known to be sensitive to treatment with ixabepilone or a pharmaceutically acceptable salt thereof. If the level of expression of the biomarker(s) in the sample from the cancer patient is substantially similar to the expression level of the biomarker(s) in the cell or tissue (e.g., tumor tissue) known to be sensitive to ixabepilone or a pharmaceutically acceptable salt thereof, then the cancer patient is predicted to be responsive to treatment with ixabepilone or a pharmaceutically acceptable salt thereof. Alternatively, if the expression level of the biomarker(s) in the sample from the cancer patient is substantially dissimilar to the expression level of the biomarker(s) in the cell or tissue (e.g., tumor tissue) known to be sensitive to ixabepilone or a pharmaceutically acceptable salt thereof, then the cancer patient is predicted to be non-responsive to treatment with ixabepilone or a pharmaceutically acceptable salt thereof.

The expression level of the biomarker(s) (e.g., HLA-DRA (SEQ ID NO: 1) or PLK2 (SEQ ID NO: 47)) in a sample from the cancer patient may also be compared to the expression level of the biomarker(s) in a cell (e.g., a cancer cell) or tissue (e.g., a tumor tissue, such as the same type of tumor as that of the cancer patient) known to be resistant to treatment with ixabepilone or a pharmaceutically acceptable salt thereof. If the expression level of the biomarker(s) in the sample from the cancer patient is substantially similar to the expression level of the biomarker(s) in the cell or tissue (e.g., a tumor tissue) known to be resistant to ixabepilone or a pharmaceutically acceptable salt thereof, then the cancer patient is predicted to be non-responsive to treatment with ixabepilone or a pharmaceutically acceptable salt thereof. Alternatively, if the expression level of the biomarker(s) in the sample from the cancer patient is substantially dissimilar to the expression level of the biomarker(s) in the cell or tissue (e.g., a tumor tissue) known to be resistant to ixabepilone or a pharmaceutically acceptable salt thereof, then the cancer patient is predicted to be responsive to treatment with ixabepilone or a pharmaceutically acceptable salt thereof.

The responsiveness of a cancer patient (e.g., a patient with cancer recurrence) to ixabepilone or a pharmaceutically acceptable salt thereof can also be predicted by comparing the expression level of a biomarker (e.g., HLA-DRA (SEQ ID NO: 1) or PLK2 (SEQ ID NO: 47)) to the expression level of the biomarker in one or more cells or tissues (e.g., from a cancer patient population, e.g., with the same cancer as that of the subject patient) known to be sensitive to treatment with ixabepilone or a pharmaceutically acceptable salt thereof, and one or more cells or tissues (e.g., from a cancer patient population) known to be resistant to treatment with ixabepilone or a pharmaceutically acceptable salt thereof. In particular, the patient may be responsive to treatment with ixabepilone or a pharmaceutically acceptable salt thereof if the expression level of the biomarker(s) is substantially similar to the expression level of the biomarker(s) in a cell or tissue (e.g., a tumor tissue) known to be sensitive to treatment with ixabepilone or a pharmaceutically acceptable salt thereof (e.g., the same type of cell or tissue from the patient), and if the expression level of the biomarker(s) is substantially dissimilar to the expression level of the biomarker(s) in a cell or tissue (e.g., a tumor tissue) known to be resistant to treatment with ixabepilone or a pharmaceutically acceptable salt thereof. Alternatively, the patient may be non-responsive to treatment with ixabepilone or a pharmaceutically acceptable salt thereof if the expression level of the biomarker(s) is substantially similar to the expression level of the biomarker(s) in a cell or tissue (e.g., a tumor tissue) known to be resistant to treatment with ixabepilone or a pharmaceutically acceptable salt thereof, and if the expression level of the biomarker(s) is substantially dissimilar to the expression level of the biomarker(s) in a cell or tissue (e.g., a tumor tissue) known to be sensitive to treatment with ixabepilone or a pharmaceutically acceptable salt thereof.

Additionally, one or more biomarkers of sensitivity (e.g., one or more of HLA-DRA (SEQ ID NOs: 1 and 2), ICAM3 (SEQ ID NO: 3), ITGB7 (SEQ ID NO: 4), CD8B (SEQ ID NO: 5), CD74 (SEQ ID NO: 6), HLA-DRB1 HLA-DRB4 HLA-DRB5 LOC100507709 LOC100507714 (SEQ ID NO: 7), IGJ (SEQ ID NO: 8), HLA-DRB1 HLA-DRB3 HLA-DRB4 LOC100507709 LOC100507714 (SEQ ID NO: 9), HLA-DPA1 (SEQ ID NOs: 10 and 12), HLA-DRB1 LOC100507709 LOC100507714 (SEQ ID NO: 11), CD28 (SEQ ID NO: 13), CD37 (SEQ ID NO: 14), NHP2 (SEQ ID NO: 15), MZB1 (SEQ ID NO: 16), ADCK3 (SEQ ID NO: 17), MYC (SEQ ID NO: 18), MEF2C (SEQ ID NOs: 19 and 24), RNASE6 (SEQ ID NO: 20), SRM (SEQ ID NO: 21), HAPLN1 (SEQ ID NO: 22), CYTIP (SEQ ID NO: 23), EIF3C EIF3CL (SEQ ID NO: 25), IQGAP2 (SEQ ID NO: 26), WBSCR22 (SEQ ID NO: 27), PIM2 (SEQ ID NO: 28), NCKAP1L (SEQ ID NO: 29), HCLS1 (SEQ ID NO: 30), MAGEA9 MAGEA9B (SEQ ID NO: 31), CTSS (SEQ ID NO: 32), GTF3A (SEQ ID NOs: 33 and 37), LCP1 (SEQ ID NO: 34), FAIM3 (SEQ ID NO: 35), HLA-DMB (SEQ ID NO: 36), RTP4 (SEQ ID NO: 38), MXI1 (SEQ ID NO: 39), SELPLG (SEQ ID NO: 40), IL10RA (SEQ ID NO: 41), POLR2H (SEQ ID NO: 42), BZW2 (SEQ ID NO: 43), CCDC88C (SEQ ID NO: 44), CORO1A (SEQ ID NO: 45), and AKAP1 (SEQ ID NO: 46)) and one or more biomarkers of resistance (e.g., one or more of PLK2 (SEQ ID NO: 47), PLXNB2 (SEQ ID NO: 48), RRAS2 (SEQ ID NO: 49 and 50), PTPLA (SEQ ID NO: 51), P2RX5-TAX1BP3 TAX1BP3 (SEQ ID NO: 52), STAT3 (SEQ ID NO: 53), PPIC (SEQ ID NO: 54), PTRF (SEQ ID NO: 55), RCN1 (SEQ ID NO: 56), ZFP36L1 (SEQ ID NO: 57), GFPT1 (SEQ ID NO: 58 AND 112), ACTN1 (SEQ ID NOs: 59 and 61), SEPT10 (SEQ ID NO: 60), COL4A1 (SEQ ID NO: 62), ERBB2IP (SEQ ID NO: 63), NNMT (SEQ ID NOs: 64 and 66), ADAM9 (SEQ ID NO: 65), TOR1AIP1 (SEQ ID NO: 67), ATP1B1 (SEQ ID NO: 68), CEBPD (SEQ ID NO: 69), FLII (SEQ ID NO: 70), FHL2 (SEQ ID NO: 71), SHC1 (SEQ ID NO: 72), SPTAN1 (SEQ ID NO: 73), CD81 (SEQ ID NO: 74), IQGAP1 (SEQ ID NO: 75), TGIF1 (SEQ ID NO: 76), PHACTR2 (SEQ ID NO: 77), COL4A2 (SEQ ID NO: 78), CEP55 (SEQ ID NO: 79), ABCC1 (SEQ ID NO: 80), PRSS23 (SEQ ID NO: 81), PTRF (SEQ ID NO: 82), TJP1 (SEQ ID NO: 83), CRK (SEQ ID NO: 84), LASP1 (SEQ ID NO: 85), PRC1 (SEQ ID NO: 86), TMEM189 TMEM189-UBE2V1 UBE2V1 (SEQ ID NO: 87), JAK1 (SEQ ID NO: 88), ACTN4 (SEQ ID NO: 89), FAM45A FAM45B (SEQ ID NO: 90), BIN1 (SEQ ID NOs: 91, 99, and 132), PPP2CB (SEQ ID NO: 92), EGFR (SEQ ID NO: 93), CNN3 (SEQ ID NO: 94), ARL6IP1 (SEQ ID NO: 95), FAIM (SEQ ID NO: 96), CDC20 (SEQ ID NO: 97), KIF23 (SEQ ID NO: 98), SDC4 (SEQ ID NO: 100), EZR (SEQ ID NO: 101), FGFR1 (SEQ ID NO: 102), LIMA1 (SEQ ID NO: 103), ITGA3 (SEQ ID NO: 104), TUBB6 (SEQ ID NO: 105), DYNLT1 (SEQ ID NO: 106), KRT7 (SEQ ID NO: 107), ANXA3 (SEQ ID NO: 108), MAPK1 (SEQ ID NO: 109), DOCK9 (SEQ ID NO: 110), ZMYM6 ZMYM6NB (SEQ ID NO: 111), MAP7D1 (SEQ ID NO: 113), BID (SEQ ID NO: 114), NMT1 (SEQ ID NO: 115), TRAM1 (SEQ ID NOs: 116 and 130), GPRC5A (SEQ ID NOS: 117 AND 166), MICALL1 (SEQ ID NO: 118), WDR1 (SEQ ID NO: 119), TRAF4 (SEQ ID NO: 120), AMOTL2 (SEQ ID NO: 121), AGRN (SEQ ID NO: 122), OBSL1 (SEQ ID NO: 123), LAPTM4B (SEQ ID NOS: 124 AND 159), MGAT4B (SEQ ID NO: 125), IQGAP1 (SEQ ID NO: 126), CTBP2 (SEQ ID NO: 127), AKR1B1 (SEQ ID NO: 128), CAPN2 (SEQ ID NO: 129), LACTB2 (SEQ ID NO: 131), PTPRK (SEQ ID NO: 133), CD24 (SEQ ID NOs: 134, 135, 144, and 157), PDLIM7 (SEQ ID NO: 136), ZNF238 (SEQ ID NO: 137), FAM129A (SEQ ID NO: 138), FAT1 (SEQ ID NO: 139), PPP4R1 (SEQ ID NO: 140), TPX2 (SEQ ID NO: 141), SOGA2 (SEQ ID NO: 142), FNTA (SEQ ID NO: 143), FNDC3B (SEQ ID NO: 145), SLC3A2 (SEQ ID NO: 146), S100A10 (SEQ ID NO: 147), RPA1 (SEQ ID NO: 148), HOXB2 (SEQ ID NO: 149), PI4KA P14KAP1 P14KAP2 (SEQ ID NO: 150), TRAF3 (SEQ ID NO: 151), BHLHE40 (SEQ ID NO: 152), CAV2 (SEQ ID NOs: 153 and 187), EPB41L2 (SEQ ID NO: 154), HDGFRP3 (SEQ ID NO: 155), AMIGO2 (SEQ ID NO: 156), CARD10 (SEQ ID NO: 158), LAPTM4B (SEQ ID NO: 159), MICA (SEQ ID NO: 160), TNFRSF10B (SEQ ID NO: 161), ZDHHC7 (SEQ ID NO: 162), SLC25A1 (SEQ ID NO: 163), ARPC5 (SEQ ID NO: 164), NF1 (SEQ ID NO: 165), GPRC5A (SEQ ID NO: 166), ACTB ACTB LOC100505829 (SEQ ID NO: 167), KRT8 (SEQ ID NO: 168), MKI67 (SEQ ID NO: 169), ANXA4 (SEQ ID NO: 170), COTL1 (SEQ ID NO: 171), LGALS3BP (SEQ ID NO: 172), PLOD2 (SEQ ID NO: 173), MPZL1 (SEQ ID NO: 174), RND3 (SEQ ID NO: 175), FZD6 (SEQ ID NO: 176), LIF (SEQ ID NO: 177), TNFRSF1A (SEQ ID NO: 178), AURKA (SEQ ID NO: 179), NR2F2 (SEQ ID NOs: 180 and 186), COPB1 (SEQ ID NO: 181), CD44 (SEQ ID NOs: 182 and 193), SMAD3 (SEQ ID NO: 183), CLPTM1 (SEQ ID NO: 184), LPHN2 (SEQ ID NO: 185), SGCE (SEQ ID NO: 188), FAM134B (SEQ ID NO: 189), IL6 (SEQ ID NO: 190), RAB32 (SEQ ID NO: 191), and FLNB (SEQ ID NO: 192)) may be used in combination to determine the responsiveness of a cancer patient (e.g., a patient with cancer recurrence) to treatment with ixabepilone or a pharmaceutically acceptable salt thereof. For example, the predicted responsiveness of a cancer patient may be determined from, e.g., the difference score, which may be defined as the difference between the mean score (i.e., mean of the expression level) of the one or more biomarkers of sensitivity of Table 1 (e.g., HLA-DRA (SEQ ID NO: 1)) and the mean score (i.e., mean of the expression level) of the one or more biomarkers of resistance of Table 2 (e.g., PLK2 (SEQ ID NO: 47)).

The difference score of the cancer patient can then be compared to the difference score in a cell (e.g., a cancer cell) or tissue (e.g., a tumor tissue) known to be sensitive or resistant to treatment with ixabepilone or a pharmaceutically acceptable salt thereof. In particular, the patient may be responsive to treatment with ixabepilone or a pharmaceutically acceptable salt thereof if the difference score is substantially similar to the difference score in a cell or tissue (e.g., a tumor tissue) known to be sensitive to treatment with ixabepilone or a pharmaceutically acceptable salt thereof. Additionally, the patient may be responsive to treatment with ixabepilone or a pharmaceutically acceptable salt thereof if the difference score is substantially dissimilar to the difference score in a cell or tissue (e.g., a tumor tissue) known to be resistant to treatment with ixabepilone or a pharmaceutically acceptable salt thereof. Alternatively, the patient may be non-responsive to treatment with ixabepilone or a pharmaceutically acceptable salt thereof if the difference score is substantially similar to the difference score in a cell or tissue (e.g., a tumor tissue) known to be resistant to treatment with ixabepilone or a pharmaceutically acceptable salt thereof. Moreover, the patient may be non-responsive to treatment with ixabepilone or a pharmaceutically acceptable salt thereof if the difference score is substantially dissimilar to the difference score in a cell or tissue (e.g., a tumor tissue) known to be sensitive to treatment with ixabepilone or a pharmaceutically acceptable salt thereof.

Additionally, the patient may be responsive to treatment with ixabepilone or a pharmaceutically acceptable salt thereof if the difference score is substantially similar to the difference score in a cell or tissue (e.g., a tumor tissue) known to be sensitive to treatment with ixabepilone or a pharmaceutically acceptable salt thereof, relative to the difference score in a cell or tissue (e.g., a tumor tissue) known to be resistant to treatment with ixabepilone or a pharmaceutically acceptable salt thereof. Also, the patient may be responsive to treatment with ixabepilone or a pharmaceutically acceptable salt thereof if the difference score is substantially dissimilar to the difference score in a cell or tissue (e.g., a tumor tissue) known to be resistant to treatment with ixabepilone or a pharmaceutically acceptable salt thereof, relative to the difference score in a cell or tissue (e.g., a tumor tissue) known to be sensitive to treatment with ixabepilone or a pharmaceutically acceptable salt thereof. Alternatively, the patient may be non-responsive to treatment with ixabepilone or a pharmaceutically acceptable salt thereof if the difference score is substantially similar to the difference score in a cell or tissue (e.g., a tumor tissue) known to be resistant to treatment with ixabepilone or a pharmaceutically acceptable salt thereof, relative to the difference score in a cell or tissue (e.g., a tumor tissue) known to be sensitive to treatment with ixabepilone or a pharmaceutically acceptable salt thereof. Furthermore, the patient may be non-responsive to treatment with ixabepilone or a pharmaceutically acceptable salt thereof if the difference score is substantially dissimilar to the difference score in a cell or tissue (e.g., a tumor tissue) known to be sensitive to treatment with ixabepilone or a pharmaceutically acceptable salt thereof, relative to the difference score in a cell or tissue (e.g., a tumor tissue) known to be resistant to treatment with ixabepilone or a pharmaceutically acceptable salt thereof.

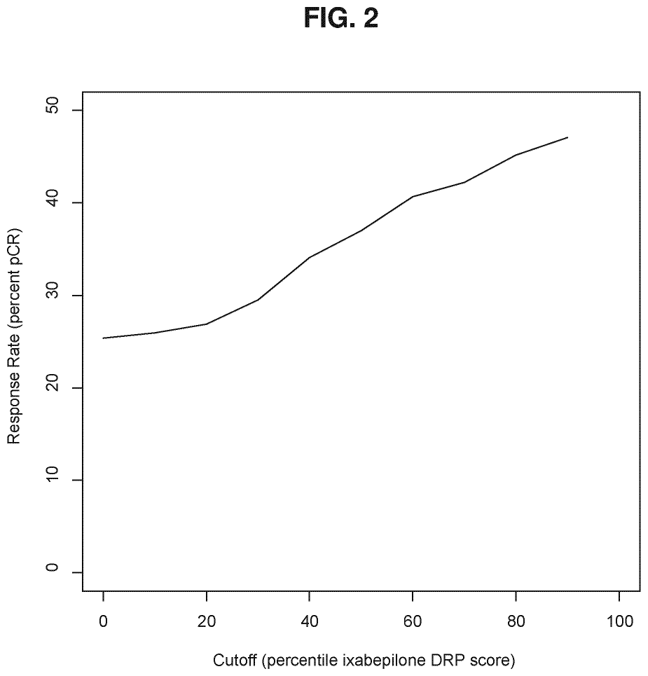

Additionally, the cancer patient (e.g., a patient with cancer recurrence) may be determined to be responsive to treatment with ixabepilone or a pharmaceutically acceptable salt thereof if the difference score is compared to a difference score from a reference population (e.g., a score determined using a tumor sample(s) from subjects diagnosed with the same type(s) of tumor as the subject) known to be sensitive to ixabepilone, in which an expression level at the 50^{th} percentile of the reference population, or the 60^{th} percentile, or the 70^{th} percentile, or the 80^{th} percentile, or the 90^{th} percentile, or greater, indicates that the sample (or the subject from whom the sample was taken) is predicted to be responsive to treatment with ixabepilone or a pharmaceutically acceptable salt thereof. The confidence of the prediction increases as the percentile level increases (e.g., an expression level above the 90^{th} percentile of a reference population indicates a greater likelihood of treatment responsiveness than an expression level at the 50^{th} percentile; see, e.g., FIG. 2). Conversely, an expression level in the tested sample of below the 50^{th} percentile of the reference population (e.g., 40^{th}, percentile, 30^{th} percentile, 20^{th} percentile, or 10^{th} percentile, or below) known to be responsive to ixabepilone indicates that the sample (or the subject from whom the sample was taken) is predicted to be non-responsive to treatment with ixabepilone or a pharmaceutically acceptable salt thereof.

Additionally, the mean score (i.e., mean of the expression level) of the one or more biomarkers of sensitivity of Table 1 and/or the mean score (i.e., mean of the expression level) of the one or more biomarkers of resistance of Table 2 can be used to predict responsiveness of a cancer patient (e.g., a patient with cancer recurrence) to ixabepilone or a pharmaceutically acceptable salt thereof. After determining the mean score of the biomarker(s) in a sample (e.g., a tumor sample) from the cancer patient, the mean score of the biomarker(s) in the sample may be compared to the mean score of the biomarker(s) in a cell (e.g., a cancer cell) or tissue (e.g., a tumor tissue) known to be sensitive to treatment with ixabepilone or a pharmaceutically acceptable salt thereof. If the mean score of the biomarker(s) in the sample from the cancer patient is substantially similar to the mean score of the biomarker(s) in the cell or tissue (e.g., a tumor tissue) known to be sensitive to ixabepilone or a pharmaceutically acceptable salt thereof, then the cancer patient is predicted to be responsive to treatment with ixabepilone or a pharmaceutically acceptable salt thereof. For example, a patient may be deemed sensitive to treatment with ixabepilone or a pharmaceutically acceptable salt thereof if the mean score of the biomarker(s) of sensitivity in a cell or tissue (e.g., a tumor tissue) is at or above a cutoff value of the 50^{th} percentile in a cell or tissue (e.g., the same type of cell or tissue as tested in the patient) obtained from a reference subject in a reference population known to be responsive to ixabepilone and having the same diagnosis as the patient, or greater (e.g., 50^{th} percentile, 60^{th} percentile, 70^{th} percentile, 80^{th} percentile, or 90^{th} percentile, or greater).

Alternatively, if the mean score of the biomarker(s) in the sample from the cancer patient is substantially dissimilar to the mean score of the biomarker(s) in the cell or tissue (e.g., a tumor tissue) known to be sensitive to ixabepilone or a pharmaceutically acceptable salt thereof, then the cancer patient is predicted to be non-responsive to treatment with ixabepilone or a pharmaceutically acceptable salt thereof. As an example, a patient may be deemed resistant to treatment with ixabepilone or a pharmaceutically acceptable salt thereof if the mean score of the biomarker(s) of sensitivity in a cell or tissue (e.g., a cancer cell or tumor tissue) is below a cutoff value of the 50^{th} percentile or less (e.g., 40^{th} percentile, 30^{th} percentile, 20^{th} percentile, or 10^{th} percentile, or less) in a cell or tissue (e.g., the same type of cell or tissue as tested in the patient) obtained from a reference subject in a reference population known to be sensitive to ixabepilone and having the same diagnosis as the patient.

The mean score (i.e., mean of the expression level) of the one or more biomarkers of sensitivity of Table 1 and/or the mean score (i.e., mean of the expression level) of the one or more biomarkers of resistance of Table 2 in a sample from the cancer patient may also be compared to the mean score of the biomarker(s) in a cell (e.g., a cancer cell) or tissue (e.g., a tumor tissue) known to be resistant to treatment with ixabepilone or a pharmaceutically acceptable salt thereof. If the mean score of the biomarker(s) in the sample from the cancer patient is substantially similar to the mean score of the biomarker(s) in the cell or tissue (e.g., a tumor tissue) known to be resistant to ixabepilone or a pharmaceutically acceptable salt thereof, then the cancer patient is predicted to be non-responsive to treatment with ixabepilone or a pharmaceutically acceptable salt thereof. Alternatively, if the mean score of the biomarker(s) in the sample from the cancer patient is substantially dissimilar to the mean score of the biomarker(s) in the cell or tissue (e.g., a tumor tissue) known to be resistant to ixabepilone or a pharmaceutically acceptable salt thereof, then the cancer patient is predicted to be responsive to treatment with ixabepilone or a pharmaceutically acceptable salt thereof.

The responsiveness of a cancer patient (e.g., a patient with cancer recurrence) to ixabepilone or a pharmaceutically acceptable salt thereof can also be predicted by comparing the mean score (i.e., mean of the expression level) of the one or more biomarkers of sensitivity of Table 1 and/or the mean score (i.e., mean of the expression level) of the one or more biomarkers of resistance of Table 2 in a sample from the cancer patient to the mean score of the biomarker(s) in one or more cells or tissues (e.g., from a cancer patient population) known to be sensitive to treatment with ixabepilone or a pharmaceutically acceptable salt thereof and one or more cells or tissues (e.g., from a cancer patient population) known to be resistant to treatment with ixabepilone or a pharmaceutically acceptable salt thereof. In particular, the patient may be responsive to treatment with ixabepilone or a pharmaceutically acceptable salt thereof if the mean score of the biomarker(s) is substantially similar to the mean score of the biomarker(s) in a cell or tissue (e.g., a tumor tissue) known to be sensitive to treatment with ixabepilone or a pharmaceutically acceptable salt thereof, relative to the mean score in a cell or tissue (e.g., a tumor tissue) known to be resistant to treatment with ixabepilone or a pharmaceutically acceptable salt thereof. Additionally, the patient may be responsive to treatment with ixabepilone or a pharmaceutically acceptable salt thereof if the mean score of the biomarker(s) is substantially dissimilar to the mean score of the biomarker(s) in a cell or tissue (e.g., a tumor tissue) known to be resistant to treatment with ixabepilone or a pharmaceutically acceptable salt thereof, relative to the mean score in a cell or tissue (e.g., a tumor tissue) known to be sensitive to treatment with ixabepilone or a pharmaceutically acceptable salt thereof. For example, a patient may be deemed sensitive to treatment with ixabepilone or a pharmaceutically acceptable salt thereof if the difference between the mean score of the biomarker(s) of sensitivity and the mean score of the biomarker(s) of resistance in the sample is below a cutoff value of the 50^{th} percentile in a cell or tissue (e.g., a tumor tissue) obtained from a reference subject in a reference population known to be resistant to ixabepilone and having the same diagnosis as the patient, or less (e.g., 40^{th} percentile, 30^{th} percentile, 20^{th} percentile, or 10^{th} percentile, or less). Alternatively, the patient may be non-responsive to treatment with ixabepilone or a pharmaceutically acceptable salt thereof if the mean score of the biomarker(s) is substantially similar to the mean score of the biomarker(s) in a cell or tissue (e.g., a tumor tissue) known to be resistant to treatment with ixabepilone or a pharmaceutically acceptable salt thereof, relative to the mean score in a cell or tissue (e.g., a tumor tissue) known to be sensitive to treatment with ixabepilone or a pharmaceutically acceptable salt thereof. Furthermore, the patient may be non-responsive to treatment with ixabepilone or a pharmaceutically acceptable salt thereof if the mean score of the biomarker(s) is substantially dissimilar to the mean score of the biomarker(s) in a cell or tissue (e.g., a tumor tissue) known to be sensitive to treatment with ixabepilone or a pharmaceutically acceptable salt thereof, relative to the mean score in a cell or tissue (e.g., a tumor tissue) known to be resistant to treatment with ixabepilone or a pharmaceutically acceptable salt thereof.

In addition, the cancer patient (e.g., a patient with cancer recurrence) may be determined to be responsive to treatment with ixabepilone or a pharmaceutically acceptable salt thereof if the mean score (i.e., mean of the expression level) of the one or more biomarkers of sensitivity of Table 1 is compared to a mean score from a reference population (e.g., a mean score determined using a tumor sample(s) from subjects diagnosed with the same type(s) of tumor as the subject), in which an expression level at the 50^{th} percentile of the reference population, or the 60^{th} percentile, or the 70^{th} percentile, or the 80^{th} percentile, or the 90^{th} percentile, or greater, indicates that the sample (or the subject from whom the sample was taken) is predicted to be responsive to treatment with ixabepilone or a pharmaceutically acceptable salt thereof. The confidence of the prediction increases as the percentile level increases (e.g., an expression level above the 90^{th} percentile of a reference population indicates a greater likelihood of treatment responsiveness than an expression level at the 50^{th} percentile; see FIG. 2). Conversely, an expression level in the tested sample of below the 50^{th} percentile of the reference population indicates that the sample (or the subject from whom the sample was taken) is predicted to be non-responsive to treatment with ixabepilone or a pharmaceutically acceptable salt thereof

Additionally, the cancer patient (e.g., a patient with cancer recurrence) may be determined to be non-responsive to treatment with ixabepilone or a pharmaceutically acceptable salt thereof if the mean score (i.e., mean of the expression level) of the one or more biomarkers of resistance of Table 2 is compared to a mean score from a reference population (e.g., a mean score determined using a tumor sample(s) from subjects diagnosed with the same type(s) of tumor as the subject), in which an expression level at the 50^{th} percentile of the reference population, or the 60^{th} percentile, or the 70^{th} percentile, or the 80^{th} percentile, or the 90^{th} percentile, or greater, indicates that the sample (or the subject from whom the sample was taken) is predicted to be non-responsive to treatment with ixabepilone or a pharmaceutically acceptable salt thereof. The confidence of the prediction increases as the percentile level increases (e.g., an expression level above the 90^{th} percentile of a reference population indicates a greater likelihood of treatment non-responsiveness than an expression level at the 50^{th} percentile).

One or more biomarkers of sensitivity and/or resistance, identified as described herein, can be used to predict responsiveness to ixabepilone or a pharmaceutically acceptable salt thereof by measuring the expression level of the biomarker(s) in a biological sample obtained from the cancer patient. A single biomarker (e.g., any of the biomarkers of Tables 1 and/or 2, such as (e.g., HLA-DRA (SEQ ID NO: 1) or PLK2 (SEQ ID NO: 47)) or a set of biomarkers (e.g., one, two, three, four, five, ten, fifteen, twenty, twenty five, or all of the biomarkers of Tables 1 and/or 2 (e.g., the top one, the top two, the top three, the top four, the top five, the top ten, the top fifteen, the top twenty, the top twenty five, or all of the biomarkers of Tables 1 and/or 2)) may be used to determine the responsiveness of a cancer patient (e.g., a patient with cancer recurrence) to ixabepilone or a pharmaceutically acceptable salt thereof. After determining the expression level of the biomarker(s) in a sample (e.g., a tumor sample) from the cancer patient, the expression level of the biomarker(s) in the sample may be compared to the expression level of the biomarker(s) in a cell (e.g., a cancer cell) or tissue (e.g., a tumor tissue) known to be sensitive to treatment with ixabepilone or a pharmaceutically acceptable salt thereof. If the expression level of the biomarker(s) in the sample from the cancer patient corresponds to the expression level of the biomarker(s) in the cell or tissue (e.g., a tumor tissue) known to be sensitive to ixabepilone or a pharmaceutically acceptable salt thereof, then the cancer patient is predicted to be responsive to treatment with ixabepilone or a pharmaceutically acceptable salt thereof. Alternatively, if the expression level of the biomarker(s) in the sample from the cancer patient is substantially dissimilar to the expression level of the biomarker(s) in the cell or tissue (e.g., a tumor tissue) known to be sensitive to ixabepilone or a pharmaceutically acceptable salt thereof, then the cancer patient is predicted to be non-responsive to treatment with ixabepilone or a pharmaceutically acceptable salt thereof.

The expression level of the biomarker(s) (e.g., HLA-DRA (SEQ ID NO: 1) or PLK2 (SEQ ID NO: 47)) in a sample from the cancer patient may also be compared to the expression level of the biomarker(s) in a cell (e.g., a cancer cell) or tissue (e.g., a tumor tissue) known to be resistant to treatment with ixabepilone or a pharmaceutically acceptable salt thereof. If the expression level of the biomarker(s) in the sample from the cancer patient corresponds to the expression level of the biomarker(s) in the cell or tissue (e.g., a tumor tissue) known to be resistant to ixabepilone or a pharmaceutically acceptable salt thereof, then the cancer patient is predicted to be non-responsive to treatment with ixabepilone or a pharmaceutically acceptable salt thereof. Alternatively, if the expression level of the biomarker(s) in the sample from the cancer patient is substantially dissimilar to the expression level of the biomarker(s) in the cell or tissue (e.g., a tumor tissue) known to be resistant to ixabepilone or a pharmaceutically acceptable salt thereof, then the cancer patient is predicted to be responsive to treatment with ixabepilone.

The responsiveness of a cancer patient (e.g., a patient with cancer recurrence) to ixabepilone or a pharmaceutically acceptable salt thereof can also be predicted by comparing the expression level of the biomarker(s) (e.g., HLA-DRA (SEQ ID NO: 1) or PLK2 (SEQ ID NO: 47)) to the expression level of the biomarker(s) in one or more cells or tissues (e.g., from a cancer patient population, such as a cancer patient population having the same diagnosis as the cancer patient) known to be sensitive to treatment with ixabepilone or a pharmaceutically acceptable salt thereof, and one or more cells or tissues (e.g., from a cancer patient population, such as a cancer patient population having the same diagnosis as the cancer patient) known to be resistant to treatment with ixabepilone or a pharmaceutically acceptable salt thereof. In particular, the patient may be responsive to treatment with ixabepilone or a pharmaceutically acceptable salt thereof if the expression level of the biomarker(s) corresponds to the expression level of the biomarker(s) in a cell or tissue (e.g., a tumor tissue) known to be sensitive to treatment with ixabepilone or a pharmaceutically acceptable salt thereof, relative to the expression level of the biomarker(s) in a cell or tissue (e.g., a tumor tissue) known to be resistant to treatment with ixabepilone or a pharmaceutically acceptable salt thereof. Moreover, the patient may be responsive to treatment with ixabepilone or a pharmaceutically acceptable salt thereof if the expression level of the biomarker(s) is substantially dissimilar to the expression level of the biomarker(s) in a cell or tissue (e.g., a tumor tissue) known to be resistant to treatment with ixabepilone or a pharmaceutically acceptable salt thereof, relative to the expression level of the biomarker(s) in a cell or tissue (e.g., a tumor tissue) known to be sensitive to treatment with ixabepilone or a pharmaceutically acceptable salt thereof. Alternatively, the patient may be non-responsive to treatment with ixabepilone or a pharmaceutically acceptable salt thereof if the expression level of the biomarker(s) corresponds to the expression level of the biomarker(s) in a cell or tissue (e.g., a tumor tissue) known to be resistant to treatment with ixabepilone or a pharmaceutically acceptable salt thereof, relative to the expression level of the biomarker(s) in a cell or tissue (e.g., a tumor tissue) known to be sensitive to treatment with ixabepilone or a pharmaceutically acceptable salt thereof. Also, the patient may be non-responsive to treatment with ixabepilone or a pharmaceutically acceptable salt thereof if the expression level of the biomarker(s) is substantially dissimilar to the expression level of the biomarker(s) in a cell or tissue (e.g., a tumor tissue) known to be sensitive to treatment with ixabepilone or a pharmaceutically acceptable salt thereof, relative to the expression level of the biomarker(s) in a cell or tissue (e.g., a tumor tissue) known to be resistant to treatment with ixabepilone or a pharmaceutically acceptable salt thereof.

Additionally, one or more biomarkers of sensitivity (e.g., one or more of HLA-DRA (SEQ ID NOs: 1 and 2), ICAM3 (SEQ ID NO: 3), ITGB7 (SEQ ID NO: 4), CD8B (SEQ ID NO: 5), CD74 (SEQ ID NO: 6), HLA-DRB1 HLA-DRB4 HLA-DRB5 LOC100507709 LOC100507714 (SEQ ID NO: 7), IGJ (SEQ ID NO: 8), HLA-DRB1 HLA-DRB3 HLA-DRB4 LOC100507709 LOC100507714 (SEQ ID NO: 9), HLA-DPA1 (SEQ ID NOs: 10 and 12), HLA-DRB1 LOC100507709 LOC100507714 (SEQ ID NO: 11), CD28 (SEQ ID NO: 13), CD37 (SEQ ID NO: 14), NHP2 (SEQ ID NO: 15), MZB1 (SEQ ID NO: 16), ADCK3 (SEQ ID NO: 17), MYC (SEQ ID NO: 18), MEF2C (SEQ ID NOs: 19 and 24), RNASE6 (SEQ ID NO: 20), SRM (SEQ ID NO: 21), HAPLN1 (SEQ ID NO: 22), CYTIP (SEQ ID NO: 23), EIF3C EIF3CL (SEQ ID NO: 25), IQGAP2 (SEQ ID NO: 26), WBSCR22 (SEQ ID NO: 27), PIM2 (SEQ ID NO: 28), NCKAP1L (SEQ ID NO: 29), HCLS1 (SEQ ID NO: 30), MAGEA9 MAGEA9B (SEQ ID NO: 31), CTSS (SEQ ID NO: 32), GTF3A (SEQ ID NOs: 33 and 37), LCP1 (SEQ ID NO: 34), FAIM3 (SEQ ID NO: 35), HLA-DMB (SEQ ID NO: 36), RTP4 (SEQ ID NO: 38), MXI1 (SEQ ID NO: 39), SELPLG (SEQ ID NO: 40), IL10RA (SEQ ID NO: 41), POLR2H (SEQ ID NO: 42), BZW2 (SEQ ID NO: 43), CCDC88C (SEQ ID NO: 44), CORO1A (SEQ ID NO: 45), and AKAP1 (SEQ ID NO: 46)) and one or more biomarkers of resistance (e.g., one or more of PLK2 (SEQ ID NO: 47), PLXNB2 (SEQ ID NO: 48), RRAS2 (SEQ ID NO: 49 and 50), PTPLA (SEQ ID NO: 51), P2RX5-TAX1BP3 TAX1BP3 (SEQ ID NO: 52), STAT3 (SEQ ID NO: 53), PPIC (SEQ ID NO: 54), PTRF (SEQ ID NO: 55), RCN1 (SEQ ID NO: 56), ZFP36L1 (SEQ ID NO: 57), GFPT1 (SEQ ID NO: 58 AND 112), ACTN1 (SEQ ID NOs: 59 and 61), SEPT10 (SEQ ID NO: 60), COL4A1 (SEQ ID NO: 62), ERBB2IP (SEQ ID NO: 63), NNMT (SEQ ID NOs: 64 and 66), ADAM9 (SEQ ID NO: 65), TOR1AIP1 (SEQ ID NO: 67), ATP1B1 (SEQ ID NO: 68), CEBPD (SEQ ID NO: 69), FLII (SEQ ID NO: 70), FHL2 (SEQ ID NO: 71), SHC1 (SEQ ID NO: 72), SPTAN1 (SEQ ID NO: 73), CD81 (SEQ ID NO: 74), IQGAP1 (SEQ ID NO: 75), TGIF1 (SEQ ID NO: 76), PHACTR2 (SEQ ID NO: 77), COL4A2 (SEQ ID NO: 78), CEP55 (SEQ ID NO: 79), ABCC1 (SEQ ID NO: 80), PRSS23 (SEQ ID NO: 81), PTRF (SEQ ID NO: 82), TJP1 (SEQ ID NO: 83), CRK (SEQ ID NO: 84), LASP1 (SEQ ID NO: 85), PRC1 (SEQ ID NO: 86), TMEM189 TMEM189-UBE2V1 UBE2V1 (SEQ ID NO: 87), JAK1 (SEQ ID NO: 88), ACTN4 (SEQ ID NO: 89), FAM45A FAM45B (SEQ ID NO: 90), BIN1 (SEQ ID NOs: 91, 99, and 132), PPP2CB (SEQ ID NO: 92), EGFR (SEQ ID NO: 93), CNN3 (SEQ ID NO: 94), ARL6IP1 (SEQ ID NO: 95), FAIM (SEQ ID NO: 96), CDC20 (SEQ ID NO: 97), KIF23 (SEQ ID NO: 98), SDC4 (SEQ ID NO: 100), EZR (SEQ ID NO: 101), FGFR1 (SEQ ID NO: 102), LIMA1 (SEQ ID NO: 103), ITGA3 (SEQ ID NO: 104), TUBB6 (SEQ ID NO: 105), DYNLT1 (SEQ ID NO: 106), KRT7 (SEQ ID NO: 107), ANXA3 (SEQ ID NO: 108), MAPK1 (SEQ ID NO: 109), DOCK9 (SEQ ID NO: 110), ZMYM6 ZMYM6NB (SEQ ID NO: 111), MAP7D1 (SEQ ID NO: 113), BID (SEQ ID NO: 114), NMT1 (SEQ ID NO: 115), TRAM1 (SEQ ID NOs: 116 and 130), GPRC5A (SEQ ID NOS: 117 AND 166), MICALL1 (SEQ ID NO: 118), WDR1 (SEQ ID NO: 119), TRAF4 (SEQ ID NO: 120), AMOTL2 (SEQ ID NO: 121), AGRN (SEQ ID NO: 122), OBSL1 (SEQ ID NO: 123), LAPTM4B (SEQ ID NOS: 124 AND 159), MGAT4B (SEQ ID NO: 125), IQGAP1 (SEQ ID NO: 126), CTBP2 (SEQ ID NO: 127), AKR1B1 (SEQ ID NO: 128), CAPN2 (SEQ ID NO: 129), LACTB2 (SEQ ID NO: 131), PTPRK (SEQ ID NO: 133), CD24 (SEQ ID NOs: 134, 135, 144, and 157), PDLIM7 (SEQ ID NO: 136), ZNF238 (SEQ ID NO: 137), FAM129A (SEQ ID NO: 138), FAT1 (SEQ ID NO: 139), PPP4R1 (SEQ ID NO: 140), TPX2 (SEQ ID NO: 141), SOGA2 (SEQ ID NO: 142), FNTA (SEQ ID NO: 143), FNDC3B (SEQ ID NO: 145), SLC3A2 (SEQ ID NO: 146), S100A10 (SEQ ID NO: 147), RPA1 (SEQ ID NO: 148), HOXB2 (SEQ ID NO: 149), PI4KA PI4KAP1 PI4KAP2 (SEQ ID NO: 150), TRAF3 (SEQ ID NO: 151), BHLHE40 (SEQ ID NO: 152), CAV2 (SEQ ID NOs: 153 and 187), EPB41L2 (SEQ ID NO: 154), HDGFRP3 (SEQ ID NO: 155), AMIGO2 (SEQ ID NO: 156), CARD10 (SEQ ID NO: 158), LAPTM4B (SEQ ID NO: 159), MICA (SEQ ID NO: 160), TNFRSF10B (SEQ ID NO: 161), ZDHHC7 (SEQ ID NO: 162), SLC25A1 (SEQ ID NO: 163), ARPC5 (SEQ ID NO: 164), NF1 (SEQ ID NO: 165), GPRC5A (SEQ ID NO: 166), ACTB ACTB LOC100505829 (SEQ ID NO: 167), KRT8 (SEQ ID NO: 168), MKI67 (SEQ ID NO: 169), ANXA4 (SEQ ID NO: 170), COTL1 (SEQ ID NO: 171), LGALS3BP (SEQ ID NO: 172), PLOD2 (SEQ ID NO: 173), MPZL1 (SEQ ID NO: 174), RND3 (SEQ ID NO: 175), FZD6 (SEQ ID NO: 176), LIF (SEQ ID NO: 177), TNFRSF1A (SEQ ID NO: 178), AURKA (SEQ ID NO: 179), NR2F2 (SEQ ID NOs: 180 and 186), COPB1 (SEQ ID NO: 181), CD44 (SEQ ID NOs: 182 and 193), SMAD3 (SEQ ID NO: 183), CLPTM1 (SEQ ID NO: 184), LPHN2 (SEQ ID NO: 185), SGCE (SEQ ID NO: 188), FAM134B (SEQ ID NO: 189), IL6 (SEQ ID NO: 190), RAB32 (SEQ ID NO: 191), and FLNB (SEQ ID NO: 192)) may be used in combination to determine the responsiveness of a cancer patient (e.g., a patient with cancer recurrence) to treatment with ixabepilone or a pharmaceutically acceptable salt thereof. For example, the predicted responsiveness of a cancer patient may be determined from, e.g., the difference score, which may be defined as the difference between the mean score (i.e., mean of the expression level) of the one or more biomarkers of sensitivity of Table 1 (e.g., HLA-DRA (SEQ ID NO: 1)) and the mean score (i.e., mean of the expression level) of the one or more biomarkers of resistance of Table 2 (e.g., PLK2 (SEQ ID NO: 47)).

The difference score of the cancer patient can then be compared to the difference score in a cell (e.g., a cancer cell) or tissue (e.g., a tumor tissue) known to be sensitive or resistant to treatment with ixabepilone or a pharmaceutically acceptable salt thereof. In particular, the patient may be responsive to treatment with ixabepilone or a pharmaceutically acceptable salt thereof if the difference score corresponds to the difference score in a cell or tissue (e.g., a tumor tissue) known to be sensitive to treatment with ixabepilone or a pharmaceutically acceptable salt thereof. Additionally, the patient may be responsive to treatment with ixabepilone or a pharmaceutically acceptable salt thereof if the difference score is substantially dissimilar to the difference score in a cell or tissue (e.g., a tumor tissue) known to be resistant to treatment with ixabepilone or a pharmaceutically acceptable salt thereof. Alternatively, the patient may be non-responsive to treatment with ixabepilone or a pharmaceutically acceptable salt thereof if the difference score corresponds to the difference score in a cell or tissue (e.g., a tumor tissue) known to be resistant to treatment with ixabepilone or a pharmaceutically acceptable salt thereof. Also, the patient may be non-responsive to treatment with ixabepilone or a pharmaceutically acceptable salt thereof if the difference score is substantially dissimilar to the difference score in a cell or tissue (e.g., a tumor tissue) known to be sensitive to treatment with ixabepilone or a pharmaceutically acceptable salt thereof.

Additionally, the patient may be responsive to treatment with ixabepilone or a pharmaceutically acceptable salt thereof if the difference score corresponds to the difference score in a cell or tissue (e.g., a tumor tissue) known to be sensitive to treatment with ixabepilone or a pharmaceutically acceptable salt thereof, relative to the difference score in a cell or tissue (e.g., a tumor tissue) known to be resistant to treatment with ixabepilone or a pharmaceutically acceptable salt thereof. Furthermore, the patient may be responsive to treatment with ixabepilone or a pharmaceutically acceptable salt thereof if the difference score is substantially dissimilar to the difference score in a cell or tissue (e.g., a tumor tissue) known to be resistant to treatment with ixabepilone or a pharmaceutically acceptable salt thereof, relative to the difference score in a cell or tissue (e.g., a tumor tissue) known to be sensitive to treatment with ixabepilone or a pharmaceutically acceptable salt thereof. Alternatively, the patient may be non-responsive to treatment with ixabepilone or a pharmaceutically acceptable salt thereof if the difference score corresponds to the difference score in a cell or tissue (e.g., a tumor tissue) known to be resistant to treatment with ixabepilone or a pharmaceutically acceptable salt thereof, relative to the difference score in a cell or tissue (e.g., a tumor tissue) known to be sensitive to treatment with ixabepilone or a pharmaceutically acceptable salt thereof. Additionally, the patient may be non-responsive to treatment with ixabepilone or a pharmaceutically acceptable salt thereof if the difference score is substantially dissimilar to the difference score in a cell or tissue (e.g., a tumor tissue) known to be sensitive to treatment with ixabepilone or a pharmaceutically acceptable salt thereof, relative to the difference score in a cell or tissue (e.g., a tumor tissue) known to be resistant to treatment with ixabepilone or a pharmaceutically acceptable salt thereof.

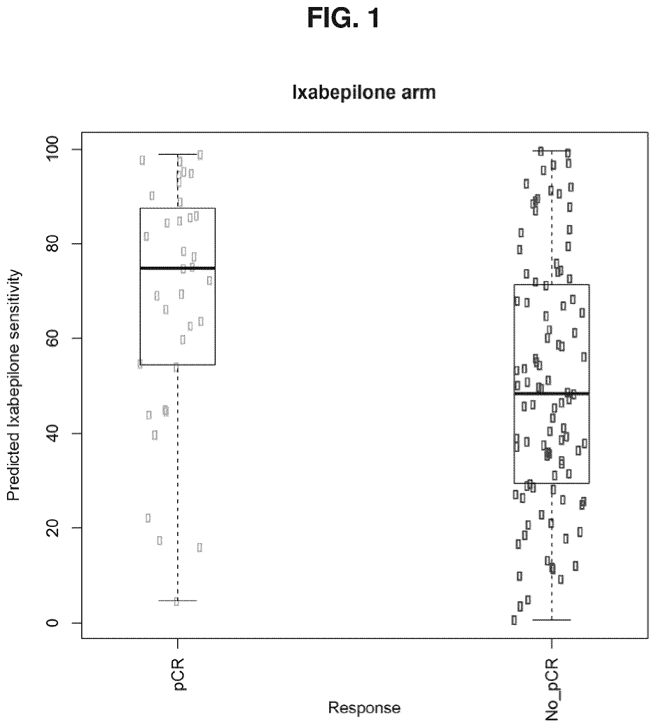

Alternatively, the patient may be determined to be responsive to treatment with ixabepilone or a pharmaceutically acceptable salt thereof if the difference score is above a cutoff value of the 50^{th} percentile in a reference population with the same diagnosis as the patient, or greater (e.g., the difference score is above a cutoff value of the 60^{th} percentile, 70^{th} percentile, 80^{th} percentile, or 90^{th} percentile, or greater; see, e.g., FIG. 1).

Preferably, the cell or tissue (e.g., a tumor tissue) known to be either sensitive or resistant to ixabepilone or a pharmaceutically acceptable salt thereof is of the same cancer type as the cancer patient with an unknown responsiveness to ixabepilone or a pharmaceutically acceptable salt thereof. For example, the cancer patient and the cell or tissue (e.g., a tumor tissue) known to be either sensitive or resistant to ixabepilone or a pharmaceutically acceptable salt thereof may both have a cancer type selected from a hematological cancer or a solid tumor, such as, e.g., breast cancer (e.g., medullary carcinoma), myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, prostate cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), ovarian cancer (e.g., ovarian adenocarcinoma or embryonal carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), ER-positive cancer, endometrial cancer, bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), metastatic cancer, oral cavity cancer, uterine cancer, testicular cancer (e.g., seminoma and embryonal carcinoma), skin cancer (e.g., squamous cell carcinoma and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, liver cancer (e.g., hepatocellular carcinoma or hepatoma), larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, and neoplasms of the central nervous system. In particular, the cancer of the patient and the cell or tissue (e.g., a tumor tissue) with known resistance or sensitivity to ixabepilone or a pharmaceutically acceptable salt thereof is, e.g., multiple myeloma, breast cancer, acute myelogenous leukemia (AML), acute lympho-blastic leukemia (ALL), chronic lymphocytic leukemia (CLL), myelodysplastic syndrome (MDS), chronic myelogenous leukemia-chronic phase (CMLCP), diffuse large B-cell lymphoma (DLBCL), cutaneous T-cell lymphoma (CTCL), peripheral T-cell lymphoma (PTCL), Hodgkin's lymphoma, hepatocellular carcinoma (HCC), cervical cancer, prostate cancer, kidney cancer, renal cell carcinoma (RCC), esophageal cancer, melanoma, glioma, pancreatic cancer, ovarian cancer, gastrointestinal stromal tumors (GIST), sarcoma, breast cancer, estrogen receptor-positive (ERpos) breast cancer, metastatic breast cancer, endometrial cancer, lung cancer, non-small cell lung carcinoma (NSCLC), mesothelioma, intestinal cancer, colon cancer, bladder cancer, adrenal cancer, gallbladder cancer, or squamous cell carcinoma of the head and neck (SCCHN). In particular, the cancer of the patient and the cell or tissue (e.g., a tumor tissue) with known resistance or sensitivity to ixabepilone or a pharmaceutically acceptable salt thereof may be estrogen receptor-positive (ER pos) breast cancer. In particular instances, the cancer of the patient and the cell or tissue (e.g., a tumor tissue) with known resistance or sensitivity to ixabepilone or a pharmaceutically acceptable salt thereof may be a metastatic form of breast cancer. The methods, devices, and kits described herein can be used to determine responsiveness of multiple types of cancer disclosed herein to ixabepilone or a pharmaceutically acceptable salt thereof.

Machine learning techniques such as Neural Networks, Support Vector Machines, K Nearest Neighbor, and Nearest Centroids may also be employed to develop models that discriminate patients sensitive to treatment with ixabepilone or a pharmaceutically acceptable salt thereof from those resistant to treatment with ixabepilone or a pharmaceutically acceptable salt thereof using biomarker expression as model variables, which assign each patient a classification as sensitive or resistant to treatment with ixabepilone or a pharmaceutically acceptable salt thereof. Machine learning techniques used to classify patients using various measurements are described in U.S. Pat. No. 5,822,715; U.S. Patent Application Publication Nos. 2003/0073083, 2005/0227266, 2005/0208512, 2005/0123945, 2003/0129629, and 2002/0006613; and in Vapnik V N. Statistical Learning Theory, John Wiley & Sons, New York, 1998; Hastie et al., 2001, The Elements of Statistical Learning: Data Mining, Inference, and Prediction, Springer, N.Y.; Agresti, 1996, An Introduction to Categorical Data Analysis, John Wiley & Sons, New York; V. Tresp et al., “Neural Network Modeling of Physiological Processes,” in Hanson S. J. et al. (Eds.), Computational Learning Theory and Natural Learning Systems 2, MIT Press, 1994, each of which are hereby incorporated by reference in their entirety.

### Biomarkers of Sensitivity and Resistance

The expression levels of one or more biomarkers of Table(s) 1 and/or 2 can be used to determine responsiveness of a cancer patient to treatment with ixabepilone or a pharmaceutically acceptable salt thereof. In certain embodiments, the biomarker(s) of sensitivity can be selected from (a) one or more of SEQ ID NOs: 1-46 from Table 1. Moreover, in certain embodiments, the biomarker(s) of resistance can be selected from (a) one or more of SEQ ID NOs: 46-193 of Table 2. In particular embodiments, at least one (e.g., at least 1, at least 5, at least 10, at least 15, at least 20, at least 25, or at least 27 (e.g., at least the top 1, at least the top 5, at least the top 10, at least the top 15, at least the top 20, at least the top 25, or at least the top 27)) of the biomarkers of sensitivity of Table 1 and/or at least one (e.g., at least 1, at least 5, at least 10, at least 15, at least 20, at least 25, or at least 27 (e.g., at least the top 1, at least the top 5, at least the top 10, at least the top 15, at least the top 20, at least the top 25, or at least the top 27)) of the biomarkers of resistance of Table 2 can be used to determine responsiveness of a cancer patient to treatment with ixabepilone or a pharmaceutically acceptable salt thereof. In more specific embodiments, one biomarker of sensitivity from Table 1 (e.g., HLA-DRA (SEQ ID NO: 1)), and/or one biomarker of resistance from Table 2 (e.g., PLK2 (SEQ ID NO: 47)) can be used to determine responsiveness of a cancer patient to treatment with ixabepilone or a pharmaceutically acceptable salt thereof. Once determined to be sensitive, the patient can be treated with ixabepilone or a pharmaceutically acceptable salt thereof.

In particular, the biomarker of SEQ ID NO: 1 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 1 may be assessed using nucleic acid amplification methods (e.g., PCR) or a device (e.g., a microarray). As is described above, the expression level of the biomarker of SEQ ID NO: 1 in the patient sample may then be compared, e.g., to the expression level of the biomarker of SEQ ID NO: 1 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 1 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 2-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 2 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 2 may be assessed using nucleic acid amplification methods (e.g., PCR) or a device (e.g., a microarray). As is described above, the expression level of the biomarker of SEQ ID NO: 2 in the patient sample may then be compared, e.g., to the expression level of the biomarker of SEQ ID NO: 2 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 2 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers SEQ ID NOs: 1, 3-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 3 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 3 may be assessed using nucleic acid amplification methods (e.g., PCR) or a device (e.g., a microarray). As is described above, the expression level of the biomarker of SEQ ID NO: 3 in the patient sample may then be compared, e.g., to the expression level of the biomarker of SEQ ID NO: 3 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 3 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1, 2, 4-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 4 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 4 may be assessed using nucleic acid amplification methods (e.g., PCR) or a device (e.g., a microarray). As is described above, the expression level of the biomarker of SEQ ID NO: 4 in the patient sample may then be compared, e.g., to the expression level of the biomarker of SEQ ID NO: 4 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 4 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-3, 5-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 5 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 5 may be assessed using nucleic acid amplification methods (e.g., PCR) or a device (e.g., a microarray). As is described above, the expression level of the biomarker of SEQ ID NO: 5 in the patient sample may then be compared, e.g., to the expression level of the biomarker of SEQ ID NO: 5 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 5 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-4, 6-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 6 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 6 may be assessed using nucleic acid amplification methods (e.g., PCR) or a device (e.g., a microarray). As is described above, the expression level of the biomarker of SEQ ID NO: 6 in the patient sample may then be compared, e.g., to the expression level of the biomarker of SEQ ID NO: 6 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 6 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-5, 7-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 7 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 7 may be assessed using nucleic acid amplification methods (e.g., PCR) or a device (e.g., a microarray). As is described above, the expression level of the biomarker of SEQ ID NO: 7 in the patient sample may then be compared, e.g., to the expression level of the biomarker of SEQ ID NO: 7 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 7 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-6, 8-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 8 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 8 may be assessed using nucleic acid amplification methods (e.g., PCR) or a device (e.g., a microarray). As is described above, the expression level of the biomarker of SEQ ID NO: 8 in the patient sample may then be compared, e.g., to the expression level of the biomarker of SEQ ID NO: 8 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 8 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-7, 9-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 9 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 9 may be assessed using nucleic acid amplification methods (e.g., PCR) or a device (e.g., a microarray). As is described above, the expression level of the biomarker of SEQ ID NO: 9 in the patient sample may then be compared, e.g., to the expression level of the biomarker of SEQ ID NO: 9 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 9 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-8, 10-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 10 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 10 may be assessed using nucleic acid amplification methods (e.g., PCR) or a device (e.g., a microarray). As is described above, the expression level of the biomarker of SEQ ID NO: 10 in the patient sample may then be compared, e.g., to the expression level of the biomarker of SEQ ID NO: 10 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 10 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-9, 11-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 11 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 11 may be assessed using nucleic acid amplification methods (e.g., PCR) or a device (e.g., a microarray). As is described above, the expression level of the biomarker of SEQ ID NO: 11 in the patient sample may then be compared, e.g., to the expression level of the biomarker of SEQ ID NO: 11 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 11 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-10, 12-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 12 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 12 may be assessed using nucleic acid amplification methods (e.g., PCR) or a device (e.g., a microarray). As is described above, the expression level of the biomarker of SEQ ID NO: 12 in the patient sample may then be compared, e.g., to the expression level of the biomarker of SEQ ID NO: 12 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 12 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-11, 13-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 13 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 13 may be assessed using nucleic acid amplification methods (e.g., PCR) or a device (e.g., a microarray). As is described above, the expression level of the biomarker of SEQ ID NO: 13 in the patient sample may then be compared, e.g., to the expression level of the biomarker of SEQ ID NO: 13 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 13 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-12, 14-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 14 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 14 may be assessed using nucleic acid amplification methods (e.g., PCR) or a device (e.g., a microarray). As is described above, the expression level of the biomarker of SEQ ID NO: 14 in the patient sample may then be compared, e.g., to the expression level of the biomarker of SEQ ID NO: 14 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 14 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-13, 15-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 15 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 15 may be assessed using nucleic acid amplification methods (e.g., PCR) or a device (e.g., a microarray). As is described above, the expression level of the biomarker of SEQ ID NO: 15 in the patient sample may then be compared, e.g., to the expression level of the biomarker of SEQ ID NO: 15 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 15 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-14, 16-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 16 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 16 may be assessed using nucleic acid amplification methods (e.g., PCR) or a device (e.g., a microarray). As is described above, the expression level of the biomarker of SEQ ID NO: 16 in the patient sample may then be compared, e.g., to the expression level of the biomarker of SEQ ID NO: 16 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 16 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-15, 17-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 17 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 17 may be assessed using nucleic acid amplification methods (e.g., PCR) or a device (e.g., a microarray). As is described above, the expression level of the biomarker of SEQ ID NO: 17 in the patient sample may then be compared, e.g., to the expression level of the biomarker of SEQ ID NO: 17 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 17 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-16, 18-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 18 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 18 may be assessed using nucleic acid amplification methods (e.g., PCR) or a device (e.g., a microarray). As is described above, the expression level of the biomarker of SEQ ID NO: 18 in the patient sample may then be compared, e.g., to the expression level of the biomarker of SEQ ID NO: 18 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 18 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-17, 19-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 19 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 19 may be assessed using nucleic acid amplification methods (e.g., PCR) or a device (e.g., a microarray). As is described above, the expression level of the biomarker of SEQ ID NO: 19 in the patient sample may then be compared, e.g., to the expression level of the biomarker of SEQ ID NO: 19 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 19 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-18, 20-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 20 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 20 may be assessed using nucleic acid amplification methods (e.g., PCR) or a device (e.g., a microarray). As is described above, the expression level of the biomarker of SEQ ID NO: 20 in the patient sample may then be compared, e.g., to the expression level of the biomarker of SEQ ID NO: 20 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 20 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-19, 21-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 21 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 21 may be assessed using nucleic acid amplification methods (e.g., PCR) or a device (e.g., a microarray). As is described above, the expression level of the biomarker of SEQ ID NO: 21 in the patient sample may then be compared, e.g., to the expression level of the biomarker of SEQ ID NO: 21 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 21 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-20, 22-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 22 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 22 may be assessed using nucleic acid amplification methods (e.g., PCR) or a device (e.g., a microarray). As is described above, the expression level of the biomarker of SEQ ID NO: 22 in the patient sample may then be compared, e.g., to the expression level of the biomarker of SEQ ID NO: 22 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 22 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-21, 23-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 23 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 23 may be assessed using nucleic acid amplification methods (e.g., PCR) or a device (e.g., a microarray). As is described above, the expression level of the biomarker of SEQ ID NO: 23 in the patient sample may then be compared, e.g., to the expression level of the biomarker of SEQ ID NO: 23 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 23 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-22, 24-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 24 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 24 may be assessed using nucleic acid amplification methods (e.g., PCR) or a device (e.g., a microarray). As is described above, the expression level of the biomarker of SEQ ID NO: 24 in the patient sample may then be compared, e.g., to the expression level of the biomarker of SEQ ID NO: 24 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 24 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-23, 25-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 25 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 25 may be assessed using nucleic acid amplification methods (e.g., PCR) or a device (e.g., a microarray). As is described above, the expression level of the biomarker of SEQ ID NO: 25 in the patient sample may then be compared, e.g., to the expression level of the biomarker of SEQ ID NO: 25 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 25 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-24, 26-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 26 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 26 may be assessed using nucleic acid amplification methods (e.g., PCR) or a device (e.g., a microarray). As is described above, the expression level of the biomarker of SEQ ID NO: 26 in the patient sample may then be compared, e.g., to the expression level of the biomarker of SEQ ID NO: 26 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 26 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-25, 27-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 27 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 27 may be assessed using nucleic acid amplification methods (e.g., PCR) or a device (e.g., a microarray). As is described above, the expression level of the biomarker of SEQ ID NO: 27 in the patient sample may then be compared, e.g., to the expression level of the biomarker of SEQ ID NO: 27 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 27 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-26, 28-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 28 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 28 may be assessed using nucleic acid amplification methods (e.g., PCR) or a device (e.g., a microarray). As is described above, the expression level of the biomarker of SEQ ID NO: 28 in the patient sample may then be compared, e.g., to the expression level of the biomarker of SEQ ID NO: 28 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 28 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-27, 29-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 29 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 29 may be assessed using nucleic acid amplification methods (e.g., PCR) or a device (e.g., a microarray). As is described above, the expression level of the biomarker of SEQ ID NO: 29 in the patient sample may then be compared, e.g., to the expression level of the biomarker of SEQ ID NO: 29 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 29 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-28, 30-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 30 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 30 may be assessed using nucleic acid amplification methods (e.g., PCR) or a device (e.g., a microarray). As is described above, the expression level of the biomarker of SEQ ID NO: 30 in the patient sample may then be compared, e.g., to the expression level of the biomarker of SEQ ID NO: 30 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 30 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-29, 31-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 31 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 31 may be assessed using nucleic acid amplification methods (e.g., PCR) or a device (e.g., a microarray). As is described above, the expression level of the biomarker of SEQ ID NO: 31 in the patient sample may then be compared, e.g., to the expression level of the biomarker of SEQ ID NO: 31 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 31 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-30, 32-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 32 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 32 may be assessed using nucleic acid amplification methods (e.g., PCR) or a device (e.g., a microarray). As is described above, the expression level of the biomarker of SEQ ID NO: 32 in the patient sample may then be compared, e.g., to the expression level of the biomarker of SEQ ID NO: 32 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 32 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-31, 33-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 33 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 33 may be assessed using nucleic acid amplification methods (e.g., PCR) or a device (e.g., a microarray). As is described above, the expression level of the biomarker of SEQ ID NO: 33 in the patient sample may then be compared, e.g., to the expression level of the biomarker of SEQ ID NO: 33 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 33 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-32, 34-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 34 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 34 may be assessed using nucleic acid amplification methods (e.g., PCR) or a device (e.g., a microarray). As is described above, the expression level of the biomarker of SEQ ID NO: 34 in the patient sample may then be compared, e.g., to the expression level of the biomarker of SEQ ID NO: 34 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 34 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-33, 35-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 35 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 35 may be assessed using nucleic acid amplification methods (e.g., PCR) or a device (e.g., a microarray). As is described above, the expression level of the biomarker of SEQ ID NO: 35 in the patient sample may then be compared, e.g., to the expression level of the biomarker of SEQ ID NO: 35 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 35 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-34, 36-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 36 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 36 may be assessed using nucleic acid amplification methods (e.g., PCR) or a device (e.g., a microarray). As is described above, the expression level of the biomarker of SEQ ID NO: 36 in the patient sample may then be compared, e.g., to the expression level of the biomarker of SEQ ID NO: 36 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 36 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-35, 37-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 37 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 37 may be assessed using nucleic acid amplification methods (e.g., PCR) or a device (e.g., a microarray). As is described above, the expression level of the biomarker of SEQ ID NO: 37 in the patient sample may then be compared, e.g., to the expression level of the biomarker of SEQ ID NO: 37 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 37 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-36, 38-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 38 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 38 may be assessed using nucleic acid amplification methods (e.g., PCR) or a device (e.g., a microarray). As is described above, the expression level of the biomarker of SEQ ID NO: 38 in the patient sample may then be compared, e.g., to the expression level of the biomarker of SEQ ID NO: 38 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 38 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-37, 39-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 39 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 39 may be assessed using nucleic acid amplification methods (e.g., PCR) or a device (e.g., a microarray). As is described above, the expression level of the biomarker of SEQ ID NO: 39 in the patient sample may then be compared, e.g., to the expression level of the biomarker of SEQ ID NO: 39 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 39 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-38, 40-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 40 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 40 may be assessed using nucleic acid amplification methods (e.g., PCR) or a device (e.g., a microarray). As is described above, the expression level of the biomarker of SEQ ID NO: 40 in the patient sample may then be compared, e.g., to the expression level of the biomarker of SEQ ID NO: 40 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 40 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-39, 41-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 41 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 41 may be assessed using nucleic acid amplification methods (e.g., PCR) or a device (e.g., a microarray). As is described above, the expression level of the biomarker of SEQ ID NO: 41 in the patient sample may then be compared, e.g., to the expression level of the biomarker of SEQ ID NO: 41 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 41 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-40, 42-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 42 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 42 may be assessed using nucleic acid amplification methods (e.g., PCR) or a device (e.g., a microarray). As is described above, the expression level of the biomarker of SEQ ID NO: 42 in the patient sample may then be compared, e.g., to the expression level of the biomarker of SEQ ID NO: 42 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 42 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-41, 43-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 43 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 43 may be assessed using nucleic acid amplification methods (e.g., PCR) or a device (e.g., a microarray). As is described above, the expression level of the biomarker of SEQ ID NO: 43 in the patient sample may then be compared, e.g., to the expression level of the biomarker of SEQ ID NO: 43 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 43 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-42, 44-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 44 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 44 may be assessed using nucleic acid amplification methods (e.g., PCR) or a device (e.g., a microarray). As is described above, the expression level of the biomarker of SEQ ID NO: 44 in the patient sample may then be compared, e.g., to the expression level of the biomarker of SEQ ID NO: 44 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 44 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-43, 45-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 45 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 45 may be assessed using nucleic acid amplification methods (e.g., PCR) or a device (e.g., a microarray). As is described above, the expression level of the biomarker of SEQ ID NO: 45 in the patient sample may then be compared, e.g., to the expression level of the biomarker of SEQ ID NO: 45 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 45 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-44, 46-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 46 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 46 may be assessed using nucleic acid amplification methods (e.g., PCR) or a device (e.g., a microarray). As is described above, the expression level of the biomarker of SEQ ID NO: 46 in the patient sample may then be compared, e.g., to the expression level of the biomarker of SEQ ID NO: 46 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 46 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-45, 47-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 47 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 47 may be assessed using nucleic acid amplification methods (e.g., PCR) or a device (e.g., a microarray). As is described above, the expression level of the biomarker of SEQ ID NO: 47 in the patient sample may then be compared, e.g., to the expression level of the biomarker of SEQ ID NO: 47 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 47 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-46, 48-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 48 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 48 may be assessed using nucleic acid amplification methods (e.g., PCR) or a device (e.g., a microarray). As is described above, the expression level of the biomarker of SEQ ID NO: 48 in the patient sample may then be compared, e.g., to the expression level of the biomarker of SEQ ID NO: 48 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 48 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-47, 49-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 49 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 49 may be assessed using nucleic acid amplification methods (e.g., PCR) or a device (e.g., a microarray). As is described above, the expression level of the biomarker of SEQ ID NO: 49 in the patient sample may then be compared, e.g., to the expression level of the biomarker of SEQ ID NO: 49 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 49 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-48, 50-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 50 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 50 may be assessed using nucleic acid amplification methods (e.g., PCR) or a device (e.g., a microarray). As is described above, the expression level of the biomarker of SEQ ID NO: 50 in the patient sample may then be compared, e.g., to the expression level of the biomarker of SEQ ID NO: 50 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 50 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-49, 51-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 51 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 51 may be assessed using nucleic acid amplification methods (e.g., PCR) or a device (e.g., a microarray). As is described above, the expression level of the biomarker of SEQ ID NO: 51 in the patient sample may then be compared, e.g., to the expression level of the biomarker of SEQ ID NO: 51 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 51 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-50, 52-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 52 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 52 may be assessed using nucleic acid amplification methods (e.g., PCR) or a device (e.g., a microarray). As is described above, the expression level of the biomarker of SEQ ID NO: 52 in the patient sample may then be compared, e.g., to the expression level of the biomarker of SEQ ID NO: 52 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 52 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-51, 53-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 53 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 53 may be assessed using nucleic acid amplification methods (e.g., PCR) or a device (e.g., a microarray). As is described above, the expression level of the biomarker of SEQ ID NO: 53 in the patient sample may then be compared, e.g., to the expression level of the biomarker of SEQ ID NO: 53 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 53 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-52, 54-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 54 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 54 may be assessed using nucleic acid amplification methods (e.g., PCR) or a device (e.g., a microarray). As is described above, the expression level of the biomarker of SEQ ID NO: 54 in the patient sample may then be compared, e.g., to the expression level of the biomarker of SEQ ID NO: 54 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 54 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-53, 55-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 55 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 55 may be assessed using nucleic acid amplification methods (e.g., PCR) or a device (e.g., a microarray). As is described above, the expression level of the biomarker of SEQ ID NO: 55 in the patient sample may then be compared, e.g., to the expression level of the biomarker of SEQ ID NO: 55 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 55 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-54, 56-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 56 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 56 may be assessed using nucleic acid amplification methods (e.g., PCR) or a device (e.g., a microarray). As is described above, the expression level of the biomarker of SEQ ID NO: 56 in the patient sample may then be compared, e.g., to the expression level of the biomarker of SEQ ID NO: 56 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 56 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-55, 57-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 57 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 57 may be assessed using nucleic acid amplification methods (e.g., PCR) or a device (e.g., a microarray). As is described above, the expression level of the biomarker of SEQ ID NO: 57 in the patient sample may then be compared, e.g., to the expression level of the biomarker of SEQ ID NO: 57 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 57 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-56, 58-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 58 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 58 may be assessed using nucleic acid amplification methods (e.g., PCR) or a device (e.g., a microarray). As is described above, the expression level of the biomarker of SEQ ID NO: 58 in the patient sample may then be compared, e.g., to the expression level of the biomarker of SEQ ID NO: 58 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 58 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-57 and 59-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 59 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 59 may be assessed using nucleic acid amplification methods (e.g., PCR) or a device (e.g., a microarray). As is described above, the expression level of the biomarker of SEQ ID NO: 59 in the patient sample may then be compared, e.g., to the expression level of the biomarker of SEQ ID NO: 59 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 59 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-58 and 60-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 60 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 60 may be assessed using nucleic acid amplification methods (e.g., PCR) or a device (e.g., a microarray). As is described above, the expression level of the biomarker of SEQ ID NO: 60 in the patient sample may then be compared, e.g., to the expression level of the biomarker of SEQ ID NO: 60 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 60 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-59 and 61-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 61 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 61 may be assessed using nucleic acid amplification methods (e.g., PCR) or a device (e.g., a microarray). As is described above, the expression level of the biomarker of SEQ ID NO: 61 in the patient sample may then be compared, e.g., to the expression level of the biomarker of SEQ ID NO: 61 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 61 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-60 and 62-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 62 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 62 may be assessed using nucleic acid amplification methods (e.g., PCR) or a device (e.g., a microarray). As is described above, the expression level of the biomarker of SEQ ID NO: 62 in the patient sample may then be compared, e.g., to the expression level of the biomarker of SEQ ID NO: 62 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 62 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-61 and 63-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 63 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 63 may be assessed using nucleic acid amplification methods (e.g., PCR) or a device (e.g., a microarray). As is described above, the expression level of the biomarker of SEQ ID NO: 63 in the patient sample may then be compared, e.g., to the expression level of the biomarker of SEQ ID NO: 63 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 63 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-62 and 64-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 64 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 64 may be assessed using nucleic acid amplification methods (e.g., PCR) or a device (e.g., a microarray). As is described above, the expression level of the biomarker of SEQ ID NO: 64 in the patient sample may then be compared, e.g., to the expression level of the biomarker of SEQ ID NO: 64 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 64 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-63 and 65-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 65 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 65 may be assessed using nucleic acid amplification methods (e.g., PCR) or a device (e.g., a microarray). As is described above, the expression level of the biomarker of SEQ ID NO: 65 in the patient sample may then be compared, e.g., to the expression level of the biomarker of SEQ ID NO: 65 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 65 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-64 and 66-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 65 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 65 may be assessed using nucleic acid amplification methods (e.g., PCR) or a device (e.g., a microarray). As is described above, the expression level of the biomarker of SEQ ID NO: 65 in the patient sample may then be compared, e.g., to the expression level of the biomarker of SEQ ID NO: 65 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 65 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-64 and 66-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 66 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 66 may be assessed using nucleic acid amplification methods (e.g., PCR) or a device (e.g., a microarray). As is described above, the expression level of the biomarker of SEQ ID NO: 66 in the patient sample may then be compared, e.g., to the expression level of the biomarker of SEQ ID NO: 66 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 66 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-65 and 67-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 67 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 67 may be assessed using nucleic acid amplification methods (e.g., PCR) or a device (e.g., a microarray). As is described above, the expression level of the biomarker of SEQ ID NO: 67 in the patient sample may then be compared, e.g., to the expression level of the biomarker of SEQ ID NO: 67 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 67 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-66 and 68-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 68 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 68 may be assessed using nucleic acid amplification methods (e.g., PCR) or a device (e.g., a microarray). As is described above, the expression level of the biomarker of SEQ ID NO: 68 in the patient sample may then be compared, e.g., to the expression level of the biomarker of SEQ ID NO: 68 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 68 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-67 and 69-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 69 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 69 may be assessed using nucleic acid amplification methods (e.g., PCR) or a device (e.g., a microarray). As is described above, the expression level of the biomarker of SEQ ID NO: 69 in the patient sample may then be compared, e.g., to the expression level of the biomarker of SEQ ID NO: 69 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 69 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-68 and 70-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 70 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 70 may be assessed using nucleic acid amplification methods (e.g., PCR) or a device (e.g., a microarray). As is described above, the expression level of the biomarker of SEQ ID NO: 70 in the patient sample may then be compared, e.g., to the expression level of the biomarker of SEQ ID NO: 70 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 70 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-69 and 71-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 71 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 71 may be assessed using nucleic acid amplification methods (e.g., PCR) or a device (e.g., a microarray). As is described above, the expression level of the biomarker of SEQ ID NO: 71 in the patient sample may then be compared, e.g., to the expression level of the biomarker of SEQ ID NO: 71 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 71 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-70 and 72-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 72 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 72 may be assessed using nucleic acid amplification methods (e.g., PCR) or a device (e.g., a microarray). As is described above, the expression level of the biomarker of SEQ ID NO: 72 in the patient sample may then be compared, e.g., to the expression level of the biomarker of SEQ ID NO: 72 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 72 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-71 and 72-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 73 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 73 may be assessed using nucleic acid amplification methods (e.g., PCR) or a device (e.g., a microarray). As is described above, the expression level of the biomarker of SEQ ID NO: 73 in the patient sample may then be compared, e.g., to the expression level of the biomarker of SEQ ID NO: 73 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 73 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-72 and 74-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 74 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 74 may be assessed using nucleic acid amplification methods (e.g., PCR) or a device (e.g., a microarray). As is described above, the expression level of the biomarker of SEQ ID NO: 74 in the patient sample may then be compared, e.g., to the expression level of the biomarker of SEQ ID NO: 74 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 74 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-73 and 75-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 75 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 75 may be assessed using nucleic acid amplification methods (e.g., PCR) or a device (e.g., a microarray). As is described above, the expression level of the biomarker of SEQ ID NO: 75 in the patient sample may then be compared, e.g., to the expression level of the biomarker of SEQ ID NO: 75 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 75 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-74 and 76-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 76 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 76 may be assessed using nucleic acid amplification methods (e.g., PCR) or a device (e.g., a microarray). As is described above, the expression level of the biomarker of SEQ ID NO: 76 in the patient sample may then be compared, e.g., to the expression level of the biomarker of SEQ ID NO: 76 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 76 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-75 and 77-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 77 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 77 may be assessed using nucleic acid amplification methods (e.g., PCR) or a device (e.g., a microarray). As is described above, the expression level of the biomarker of SEQ ID NO: 77 in the patient sample may then be compared, e.g., to the expression level of the biomarker of SEQ ID NO: 77 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 77 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-76 and 78-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 78 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 78 may be assessed using nucleic acid amplification methods (e.g., PCR) or a device (e.g., a microarray). As is described above, the expression level of the biomarker of SEQ ID NO: 78 in the patient sample may then be compared, e.g., to the expression level of the biomarker of SEQ ID NO: 78 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 78 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-77 and 79-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 79 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 79 may be assessed using nucleic acid amplification methods (e.g., PCR) or a device (e.g., a microarray). As is described above, the expression level of the biomarker of SEQ ID NO: 79 in the patient sample may then be compared, e.g., to the expression level of the biomarker of SEQ ID NO: 79 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 79 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-78 and 80-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 80 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 80 may be assessed using nucleic acid amplification methods (e.g., PCR) or a device (e.g., a microarray). As is described above, the expression level of the biomarker of SEQ ID NO: 80 in the patient sample may then be compared, e.g., to the expression level of the biomarker of SEQ ID NO: 80 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 80 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-79 and 81-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 81 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 81 may be assessed using nucleic acid amplification methods (e.g., PCR) or a device (e.g., a microarray). As is described above, the expression level of the biomarker of SEQ ID NO: 81 in the patient sample may then be compared, e.g., to the expression level of the biomarker of SEQ ID NO: 81 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 81 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-80 and 82-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 82 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 82 may be assessed using nucleic acid amplification methods (e.g., PCR) or a device (e.g., a microarray). As is described above, the expression level of the biomarker of SEQ ID NO: 82 in the patient sample may then be compared, e.g., to the expression level of the biomarker of SEQ ID NO: 82 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 82 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-81 and 83-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 83 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 83 may be assessed using nucleic acid amplification methods (e.g., PCR) or a device (e.g., a microarray). As is described above, the expression level of the biomarker of SEQ ID NO: 83 in the patient sample may then be compared, e.g., to the expression level of the biomarker of SEQ ID NO: 83 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 83 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-82 and 84-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 84 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 84 may be assessed using nucleic acid amplification methods (e.g., PCR) or a device (e.g., a microarray). As is described above, the expression level of the biomarker of SEQ ID NO: 84 in the patient sample may then be compared, e.g., to the expression level of the biomarker of SEQ ID NO: 84 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 84 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-83 and 85-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 85 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 85 may be assessed using nucleic acid amplification methods (e.g., PCR) or a device (e.g., a microarray). As is described above, the expression level of the biomarker of SEQ ID NO: 85 in the patient sample may then be compared, e.g., to the expression level of the biomarker of SEQ ID NO: 85 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 85 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-84 and 86-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 86 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 86 may be assessed using nucleic acid amplification methods (e.g., PCR) or a device (e.g., a microarray). As is described above, the expression level of the biomarker of SEQ ID NO: 86 in the patient sample may then be compared, e.g., to the expression level of the biomarker of SEQ ID NO: 86 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 86 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-85 and 87-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 87 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 87 may be assessed using nucleic acid amplification methods (e.g., PCR) or a device (e.g., a microarray). As is described above, the expression level of the biomarker of SEQ ID NO: 87 in the patient sample may then be compared, e.g., to the expression level of the biomarker of SEQ ID NO: 87 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 87 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-86 and 88-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 88 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 88 may be assessed using nucleic acid amplification methods (e.g., PCR) or a device (e.g., a microarray). As is described above, the expression level of the biomarker of SEQ ID NO: 88 in the patient sample may then be compared, e.g., to the expression level of the biomarker of SEQ ID NO: 88 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 88 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-87 and 89-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 89 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 89 may be assessed using nucleic acid amplification methods (e.g., PCR) or a device (e.g., a microarray). As is described above, the expression level of the biomarker of SEQ ID NO: 89 in the patient sample may then be compared, e.g., to the expression level of the biomarker of SEQ ID NO: 89 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 89 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-88 and 90-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 90 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 90 may be assessed using nucleic acid amplification methods (e.g., PCR) or a device (e.g., a microarray). As is described above, the expression level of the biomarker of SEQ ID NO: 90 in the patient sample may then be compared, e.g., to the expression level of the biomarker of SEQ ID NO: 90 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 90 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-89 and 91-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 91 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 91 may be assessed using nucleic acid amplification methods (e.g., PCR) or a device (e.g., a microarray). As is described above, the expression level of the biomarker of SEQ ID NO: 91 in the patient sample may then be compared, e.g., to the expression level of the biomarker of SEQ ID NO: 91 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 91 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-90 and 92-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 92 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 92 may be assessed using nucleic acid amplification methods (e.g., PCR) or a device (e.g., a microarray). As is described above, the expression level of the biomarker of SEQ ID NO: 92 in the patient sample may then be compared, e.g., to the expression level of the biomarker of SEQ ID NO: 92 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 92 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-91 and 93-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 93 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 93 may be assessed using nucleic acid amplification methods (e.g., PCR) or a device (e.g., a microarray). As is described above, the expression level of the biomarker of SEQ ID NO: 93 in the patient sample may then be compared, e.g., to the expression level of the biomarker of SEQ ID NO: 93 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 93 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-92 and 94-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 94 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 94 may be assessed using nucleic acid amplification methods (e.g., PCR) or a device (e.g., a microarray). As is described above, the expression level of the biomarker of SEQ ID NO: 94 in the patient sample may then be compared, e.g., to the expression level of the biomarker of SEQ ID NO: 94 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 94 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-93 and 95-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 95 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 95 may be assessed using nucleic acid amplification methods (e.g., PCR) or a device (e.g., a microarray). As is described above, the expression level of the biomarker of SEQ ID NO: 95 in the patient sample may then be compared, e.g., to the expression level of the biomarker of SEQ ID NO: 95 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 95 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-94 and 96-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 96 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 96 may be assessed using nucleic acid amplification methods (e.g., PCR) or a device (e.g., a microarray). As is described above, the expression level of the biomarker of SEQ ID NO: 96 in the patient sample may then be compared, e.g., to the expression level of the biomarker of SEQ ID NO: 96 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 96 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-95 and 97-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 97 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 97 may be assessed using nucleic acid amplification methods (e.g., PCR) or a device (e.g., a microarray). As is described above, the expression level of the biomarker of SEQ ID NO: 97 in the patient sample may then be compared, e.g., to the expression level of the biomarker of SEQ ID NO: 97 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 97 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-96 and 98-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 98 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 98 may be assessed using nucleic acid amplification methods (e.g., PCR) or a device (e.g., a microarray). As is described above, the expression level of the biomarker of SEQ ID NO: 98 in the patient sample may then be compared, e.g., to the expression level of the biomarker of SEQ ID NO: 98 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 98 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-97 and 99-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 99 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 99 may be assessed using nucleic acid amplification methods (e.g., PCR) or a device (e.g., a microarray). As is described above, the expression level of the biomarker of SEQ ID NO: 99 in the patient sample may then be compared, e.g., to the expression level of the biomarker of SEQ ID NO: 99 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 99 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-98 and 100-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 100 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 100 may be assessed using nucleic acid amplification methods (e.g., PCR) or a device (e.g., a microarray). As is described above, the expression level of the biomarker of SEQ ID NO: 100 in the patient sample may then be compared, e.g., to the expression level of the biomarker of SEQ ID NO: 100 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 100 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-99 and 101-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

In particular, the biomarker of SEQ ID NO: 101 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 101 may be assessed using nucleic acid amplification methods (e.g., PCR) or a device (e.g., a microarray). As is described above, the expression level of the biomarker of SEQ ID NO: 101 in the patient sample may then be compared, e.g., to the expression level of the biomarker of SEQ ID NO: 101 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 101 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-100 and 102-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 102 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 102 may be assessed using nucleic acid amplification methods (e.g., PCR) or a device (e.g., a microarray). As is described above, the expression level of the biomarker of SEQ ID NO: 102 in the patient sample may then be compared, e.g., to the expression level of the biomarker of SEQ ID NO: 102 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 102 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-101 and 103-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 103 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 103 may be assessed using nucleic acid amplification methods (e.g., PCR) or a device (e.g., a microarray). As is described above, the expression level of the biomarker of SEQ ID NO: 103 in the patient sample may then be compared, e.g., to the expression level of the biomarker of SEQ ID NO: 103 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 103 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-102 and 104-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 104 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 104 may be assessed using nucleic acid amplification methods (e.g., PCR) or a device (e.g., a microarray). As is described above, the expression level of the biomarker of SEQ ID NO: 104 in the patient sample may then be compared, e.g., to the expression level of the biomarker of SEQ ID NO: 104 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 104 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-103 and 105-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 105 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 105 may be assessed using nucleic acid amplification methods (e.g., PCR) or a device (e.g., a microarray). As is described above, the expression level of the biomarker of SEQ ID NO: 105 in the patient sample may then be compared, e.g., to the expression level of the biomarker of SEQ ID NO: 105 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 105 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-104 and 106-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 106 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 106 may be assessed using nucleic acid amplification methods (e.g., PCR) or a device (e.g., a microarray). As is described above, the expression level of the biomarker of SEQ ID NO: 106 in the patient sample may then be compared, e.g., to the expression level of the biomarker of SEQ ID NO: 106 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 106 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-105 and 107-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 107 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 107 may be assessed using nucleic acid amplification methods (e.g., PCR) or a device (e.g., a microarray). As is described above, the expression level of the biomarker of SEQ ID NO: 107 in the patient sample may then be compared, e.g., to the expression level of the biomarker of SEQ ID NO: 107 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 107 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-106 and 108-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 108 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 108 may be assessed using nucleic acid amplification methods (e.g., PCR) or a device (e.g., a microarray). As is described above, the expression level of the biomarker of SEQ ID NO: 108 in the patient sample may then be compared, e.g., to the expression level of the biomarker of SEQ ID NO: 108 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 108 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-107 and 109-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 109 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 109 may be assessed using nucleic acid amplification methods (e.g., PCR) or a device (e.g., a microarray). As is described above, the expression level of the biomarker of SEQ ID NO: 109 in the patient sample may then be compared, e.g., to the expression level of the biomarker of SEQ ID NO: 109 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 109 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-108 and 110-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 110 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 110 may be assessed using nucleic acid amplification methods (e.g., PCR) or a device (e.g., a microarray). As is described above, the expression level of the biomarker of SEQ ID NO: 110 in the patient sample may then be compared, e.g., to the expression level of the biomarker of SEQ ID NO: 110 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 110 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-109 and 111-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 111 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 111 may be assessed using nucleic acid amplification methods (e.g., PCR) or a device (e.g., a microarray). As is described above, the expression level of the biomarker of SEQ ID NO: 111 in the patient sample may then be compared, e.g., to the expression level of the biomarker of SEQ ID NO: 111 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 111 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-110 and 112-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 112 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 112 may be assessed using nucleic acid amplification methods (e.g., PCR) or a device (e.g., a microarray). As is described above, the expression level of the biomarker of SEQ ID NO: 112 in the patient sample may then be compared, e.g., to the expression level of the biomarker of SEQ ID NO: 112 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 112 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-111 and 113-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 113 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 113 may be assessed using nucleic acid amplification methods (e.g., PCR) or a device (e.g., a microarray). As is described above, the expression level of the biomarker of SEQ ID NO: 113 in the patient sample may then be compared, e.g., to the expression level of the biomarker of SEQ ID NO: 113 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 113 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-112 and 114-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 114 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 114 may be assessed using nucleic acid amplification methods (e.g., PCR) or a device (e.g., a microarray). As is described above, the expression level of the biomarker of SEQ ID NO: 114 in the patient sample may then be compared, e.g., to the expression level of the biomarker of SEQ ID NO: 114 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 114 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-113 and 115-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 115 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 115 may be assessed using nucleic acid amplification methods (e.g., PCR) or a device (e.g., a microarray). As is described above, the expression level of the biomarker of SEQ ID NO: 115 in the patient sample may then be compared, e.g., to the expression level of the biomarker of SEQ ID NO: 115 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 115 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-114 and 116-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 116 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 116 may be assessed using nucleic acid amplification methods (e.g., PCR) or a device (e.g., a microarray). As is described above, the expression level of the biomarker of SEQ ID NO: 116 in the patient sample may then be compared, e.g., to the expression level of the biomarker of SEQ ID NO: 116 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 116 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-115 and 117-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 117 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 117 may be assessed using nucleic acid amplification methods (e.g., PCR) or a device (e.g., a microarray). As is described above, the expression level of the biomarker of SEQ ID NO: 117 in the patient sample may then be compared, e.g., to the expression level of the biomarker of SEQ ID NO: 117 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 117 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-116 and 118-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 118 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 118 may be assessed using nucleic acid amplification methods (e.g., PCR) or a device (e.g., a microarray). As is described above, the expression level of the biomarker of SEQ ID NO: 118 in the patient sample may then be compared, e.g., to the expression level of the biomarker of SEQ ID NO: 118 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 118 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-117 and 119-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 119 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 119 may be assessed using nucleic acid amplification methods (e.g., PCR) or a device (e.g., a microarray). As is described above, the expression level of the biomarker of SEQ ID NO: 119 in the patient sample may then be compared, e.g., to the expression level of the biomarker of SEQ ID NO: 119 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 119 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-118 and 120-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 120 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 120 may be assessed using nucleic acid amplification methods (e.g., PCR) or a device (e.g., a microarray). As is described above, the expression level of the biomarker of SEQ ID NO: 120 in the patient sample may then be compared, e.g., to the expression level of the biomarker of SEQ ID NO: 120 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 120 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-119 and 121-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 121 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 121 may be assessed using nucleic acid amplification methods (e.g., PCR) or a device (e.g., a microarray). As is described above, the expression level of the biomarker of SEQ ID NO: 121 in the patient sample may then be compared, e.g., to the expression level of the biomarker of SEQ ID NO: 121 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 121 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-120 and 122-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 122 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 122 may be assessed using nucleic acid amplification methods (e.g., PCR) or a device (e.g., a microarray). As is described above, the expression level of the biomarker of SEQ ID NO: 122 in the patient sample may then be compared, e.g., to the expression level of the biomarker of SEQ ID NO: 122 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 122 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-121 and 123-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 123 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 123 may be assessed using nucleic acid amplification methods (e.g., PCR) or a device (e.g., a microarray). As is described above, the expression level of the biomarker of SEQ ID NO: 123 in the patient sample may then be compared, e.g., to the expression level of the biomarker of SEQ ID NO: 123 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 123 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-122 and 124-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 124 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 124 may be assessed using nucleic acid amplification methods (e.g., PCR) or a device (e.g., a microarray). As is described above, the expression level of the biomarker of SEQ ID NO: 124 in the patient sample may then be compared, e.g., to the expression level of the biomarker of SEQ ID NO: 124 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 124 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-123 and 125-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 125 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 125 may be assessed using nucleic acid amplification methods (e.g., PCR) or a device (e.g., a microarray). As is described above, the expression level of the biomarker of SEQ ID NO: 125 in the patient sample may then be compared, e.g., to the expression level of the biomarker of SEQ ID NO: 125 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 125 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-124 and 126-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 126 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 126 may be assessed using nucleic acid amplification methods (e.g., PCR) or a device (e.g., a microarray). As is described above, the expression level of the biomarker of SEQ ID NO: 126 in the patient sample may then be compared, e.g., to the expression level of the biomarker of SEQ ID NO: 126 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 126 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-125 and 127-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 127 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 127 may be assessed using nucleic acid amplification methods (e.g., PCR) or a device (e.g., a microarray). As is described above, the expression level of the biomarker of SEQ ID NO: 127 in the patient sample may then be compared, e.g., to the expression level of the biomarker of SEQ ID NO: 127 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 127 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-126 and 128-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 128 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 128 may be assessed using nucleic acid amplification methods (e.g., PCR) or a device (e.g., a microarray). As is described above, the expression level of the biomarker of SEQ ID NO: 128 in the patient sample may then be compared, e.g., to the expression level of the biomarker of SEQ ID NO: 128 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 128 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-127 and 129-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 129 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 129 may be assessed using nucleic acid amplification methods (e.g., PCR) or a device (e.g., a microarray). As is described above, the expression level of the biomarker of SEQ ID NO: 129 in the patient sample may then be compared, e.g., to the expression level of the biomarker of SEQ ID NO: 129 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 129 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-128 and 130-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 130 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 130 may be assessed using nucleic acid amplification methods (e.g., PCR) or a device (e.g., a microarray). As is described above, the expression level of the biomarker of SEQ ID NO: 130 in the patient sample may then be compared, e.g., to the expression level of the biomarker of SEQ ID NO: 130 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 130 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-129 and 131-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 131 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 131 may be assessed using nucleic acid amplification methods (e.g., PCR) or a device (e.g., a microarray). As is described above, the expression level of the biomarker of SEQ ID NO: 131 in the patient sample may then be compared, e.g., to the expression level of the biomarker of SEQ ID NO: 131 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 131 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-130 and 132-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 132 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 132 may be assessed using nucleic acid amplification methods (e.g., PCR) or a device (e.g., a microarray). As is described above, the expression level of the biomarker of SEQ ID NO: 132 in the patient sample may then be compared, e.g., to the expression level of the biomarker of SEQ ID NO: 132 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 132 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-131 and 133-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 133 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 133 may be assessed using nucleic acid amplification methods (e.g., PCR) or a device (e.g., a microarray). As is described above, the expression level of the biomarker of SEQ ID NO: 133 in the patient sample may then be compared, e.g., to the expression level of the biomarker of SEQ ID NO: 133 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 133 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-132 and 134-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 134 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 134 may be assessed using nucleic acid amplification methods (e.g., PCR) or a device (e.g., a microarray). As is described above, the expression level of the biomarker of SEQ ID NO: 134 in the patient sample may then be compared, e.g., to the expression level of the biomarker of SEQ ID NO: 134 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 134 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-133 and 135-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 135 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 135 may be assessed using nucleic acid amplification methods (e.g., PCR) or a device (e.g., a microarray). As is described above, the expression level of the biomarker of SEQ ID NO: 135 in the patient sample may then be compared, e.g., to the expression level of the biomarker of SEQ ID NO: 135 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 135 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-134 and 136-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 136 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 136 may be assessed using nucleic acid amplification methods (e.g., PCR) or a device (e.g., a microarray). As is described above, the expression level of the biomarker of SEQ ID NO: 136 in the patient sample may then be compared, e.g., to the expression level of the biomarker of SEQ ID NO: 136 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 136 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-135 and 137-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 137 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 137 may be assessed using nucleic acid amplification methods (e.g., PCR) or a device (e.g., a microarray). As is described above, the expression level of the biomarker of SEQ ID NO: 137 in the patient sample may then be compared, e.g., to the expression level of the biomarker of SEQ ID NO: 137 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 137 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-136 and 138-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 138 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 138 may be assessed using nucleic acid amplification methods (e.g., PCR) or a device (e.g., a microarray). As is described above, the expression level of the biomarker of SEQ ID NO: 138 in the patient sample may then be compared, e.g., to the expression level of the biomarker of SEQ ID NO: 138 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 138 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-137 and 139-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 139 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 139 may be assessed using nucleic acid amplification methods (e.g., PCR) or a device (e.g., a microarray). As is described above, the expression level of the biomarker of SEQ ID NO: 139 in the patient sample may then be compared, e.g., to the expression level of the biomarker of SEQ ID NO: 139 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 139 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-138 and 140-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 140 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 140 may be assessed using nucleic acid amplification methods (e.g., PCR) or a device (e.g., a microarray). As is described above, the expression level of the biomarker of SEQ ID NO: 140 in the patient sample may then be compared, e.g., to the expression level of the biomarker of SEQ ID NO: 140 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 140 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-139 and 141-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 141 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 141 may be assessed using nucleic acid amplification methods (e.g., PCR) or a device (e.g., a microarray). As is described above, the expression level of the biomarker of SEQ ID NO: 141 in the patient sample may then be compared, e.g., to the expression level of the biomarker of SEQ ID NO: 141 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 141 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-140 and 142-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 142 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 142 may be assessed using nucleic acid amplification methods (e.g., PCR) or a device (e.g., a microarray). As is described above, the expression level of the biomarker of SEQ ID NO: 142 in the patient sample may then be compared, e.g., to the expression level of the biomarker of SEQ ID NO: 142 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 142 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-141 and 143-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 143 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 143 may be assessed using nucleic acid amplification methods (e.g., PCR) or a device (e.g., a microarray). As is described above, the expression level of the biomarker of SEQ ID NO: 143 in the patient sample may then be compared, e.g., to the expression level of the biomarker of SEQ ID NO: 143 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 143 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-142 and 144-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 144 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 144 may be assessed using nucleic acid amplification methods (e.g., PCR) or a device (e.g., a microarray). As is described above, the expression level of the biomarker of SEQ ID NO: 144 in the patient sample may then be compared, e.g., to the expression level of the biomarker of SEQ ID NO: 144 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 144 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-143 and 145-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 145 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 145 may be assessed using nucleic acid amplification methods (e.g., PCR) or a device (e.g., a microarray). As is described above, the expression level of the biomarker of SEQ ID NO: 145 in the patient sample may then be compared, e.g., to the expression level of the biomarker of SEQ ID NO: 145 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 145 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-144 and 146-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 146 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 146 may be assessed using nucleic acid amplification methods (e.g., PCR) or a device (e.g., a microarray). As is described above, the expression level of the biomarker of SEQ ID NO: 146 in the patient sample may then be compared, e.g., to the expression level of the biomarker of SEQ ID NO: 146 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 146 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-145 and 147-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 147 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 147 may be assessed using nucleic acid amplification methods (e.g., PCR) or a device (e.g., a microarray). As is described above, the expression level of the biomarker of SEQ ID NO: 147 in the patient sample may then be compared, e.g., to the expression level of the biomarker of SEQ ID NO: 147 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 147 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-146 and 148-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 148 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 148 may be assessed using nucleic acid amplification methods (e.g., PCR) or a device (e.g., a microarray). As is described above, the expression level of the biomarker of SEQ ID NO: 148 in the patient sample may then be compared, e.g., to the expression level of the biomarker of SEQ ID NO: 148 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 148 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-147 and 149-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 149 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 149 may be assessed using nucleic acid amplification methods (e.g., PCR) or a device (e.g., a microarray). As is described above, the expression level of the biomarker of SEQ ID NO: 149 in the patient sample may then be compared, e.g., to the expression level of the biomarker of SEQ ID NO: 149 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 149 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-148 and 150-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 150 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 150 may be assessed using nucleic acid amplification methods (e.g., PCR) or a device (e.g., a microarray). As is described above, the expression level of the biomarker of SEQ ID NO: 150 in the patient sample may then be compared, e.g., to the expression level of the biomarker of SEQ ID NO: 150 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 150 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-149 and 151-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 151 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 151 may be assessed using nucleic acid amplification methods (e.g., PCR) or a device (e.g., a microarray). As is described above, the expression level of the biomarker of SEQ ID NO: 151 in the patient sample may then be compared, e.g., to the expression level of the biomarker of SEQ ID NO: 151 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 151 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-150 and 152-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 152 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 152 may be assessed using nucleic acid amplification methods (e.g., PCR) or a device (e.g., a microarray). As is described above, the expression level of the biomarker of SEQ ID NO: 152 in the patient sample may then be compared, e.g., to the expression level of the biomarker of SEQ ID NO: 152 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 152 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-151 and 153-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 153 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 153 may be assessed using nucleic acid amplification methods (e.g., PCR) or a device (e.g., a microarray). As is described above, the expression level of the biomarker of SEQ ID NO: 153 in the patient sample may then be compared, e.g., to the expression level of the biomarker of SEQ ID NO: 153 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 153 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-152 and 153-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 154 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 154 may be assessed using nucleic acid amplification methods (e.g., PCR) or a device (e.g., a microarray). As is described above, the expression level of the biomarker of SEQ ID NO: 154 in the patient sample may then be compared, e.g., to the expression level of the biomarker of SEQ ID NO: 154 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 154 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-153 and 155-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 155 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 155 may be assessed using nucleic acid amplification methods (e.g., PCR) or a device (e.g., a microarray). As is described above, the expression level of the biomarker of SEQ ID NO: 155 in the patient sample may then be compared, e.g., to the expression level of the biomarker of SEQ ID NO: 155 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 155 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-154 and 156-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 156 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 156 may be assessed using nucleic acid amplification methods (e.g., PCR) or a device (e.g., a microarray). As is described above, the expression level of the biomarker of SEQ ID NO: 156 in the patient sample may then be compared, e.g., to the expression level of the biomarker of SEQ ID NO: 156 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 156 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-155 and 157-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 157 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 157 may be assessed using nucleic acid amplification methods (e.g., PCR) or a device (e.g., a microarray). As is described above, the expression level of the biomarker of SEQ ID NO: 157 in the patient sample may then be compared, e.g., to the expression level of the biomarker of SEQ ID NO: 157 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 157 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-156 and 158-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 158 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 158 may be assessed using nucleic acid amplification methods (e.g., PCR) or a device (e.g., a microarray). As is described above, the expression level of the biomarker of SEQ ID NO: 158 in the patient sample may then be compared, e.g., to the expression level of the biomarker of SEQ ID NO: 158 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 158 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-157 and 159-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 159 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 159 may be assessed using nucleic acid amplification methods (e.g., PCR) or a device (e.g., a microarray). As is described above, the expression level of the biomarker of SEQ ID NO: 159 in the patient sample may then be compared, e.g., to the expression level of the biomarker of SEQ ID NO: 159 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 159 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-158 and 160-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 160 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 160 may be assessed using nucleic acid amplification methods (e.g., PCR) or a device (e.g., a microarray). As is described above, the expression level of the biomarker of SEQ ID NO: 160 in the patient sample may then be compared, e.g., to the expression level of the biomarker of SEQ ID NO: 160 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 160 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-159 and 161-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 161 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 161 may be assessed using nucleic acid amplification methods (e.g., PCR) or a device (e.g., a microarray). As is described above, the expression level of the biomarker of SEQ ID NO: 161 in the patient sample may then be compared, e.g., to the expression level of the biomarker of SEQ ID NO: 161 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 161 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-160 and 162-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 162 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 162 may be assessed using nucleic acid amplification methods (e.g., PCR) or a device (e.g., a microarray). As is described above, the expression level of the biomarker of SEQ ID NO: 162 in the patient sample may then be compared, e.g., to the expression level of the biomarker of SEQ ID NO: 162 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 162 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-161 and 163-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 163 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 163 may be assessed using nucleic acid amplification methods (e.g., PCR) or a device (e.g., a microarray). As is described above, the expression level of the biomarker of SEQ ID NO: 163 in the patient sample may then be compared, e.g., to the expression level of the biomarker of SEQ ID NO: 163 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 163 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-162 and 164-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 164 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 164 may be assessed using nucleic acid amplification methods (e.g., PCR) or a device (e.g., a microarray). As is described above, the expression level of the biomarker of SEQ ID NO: 164 in the patient sample may then be compared, e.g., to the expression level of the biomarker of SEQ ID NO: 164 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 164 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-163 and 165-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 165 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 165 may be assessed using nucleic acid amplification methods (e.g., PCR) or a device (e.g., a microarray). As is described above, the expression level of the biomarker of SEQ ID NO: 165 in the patient sample may then be compared, e.g., to the expression level of the biomarker of SEQ ID NO: 165 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 165 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-164 and 166-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 166 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 166 may be assessed using nucleic acid amplification methods (e.g., PCR) or a device (e.g., a microarray). As is described above, the expression level of the biomarker of SEQ ID NO: 166 in the patient sample may then be compared, e.g., to the expression level of the biomarker of SEQ ID NO: 166 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 166 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-165 and 167-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 167 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 167 may be assessed using nucleic acid amplification methods (e.g., PCR) or a device (e.g., a microarray). As is described above, the expression level of the biomarker of SEQ ID NO: 167 in the patient sample may then be compared, e.g., to the expression level of the biomarker of SEQ ID NO: 167 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 167 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-166 and 168-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 168 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 168 may be assessed using nucleic acid amplification methods (e.g., PCR) or a device (e.g., a microarray). As is described above, the expression level of the biomarker of SEQ ID NO: 168 in the patient sample may then be compared, e.g., to the expression level of the biomarker of SEQ ID NO: 168 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 168 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-167 and 169-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 169 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 169 may be assessed using nucleic acid amplification methods (e.g., PCR) or a device (e.g., a microarray). As is described above, the expression level of the biomarker of SEQ ID NO: 169 in the patient sample may then be compared, e.g., to the expression level of the biomarker of SEQ ID NO: 169 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 169 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-168 and 170-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 170 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 170 may be assessed using nucleic acid amplification methods (e.g., PCR) or a device (e.g., a microarray). As is described above, the expression level of the biomarker of SEQ ID NO: 170 in the patient sample may then be compared, e.g., to the expression level of the biomarker of SEQ ID NO: 170 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 170 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-169 and 171-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 171 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 171 may be assessed using nucleic acid amplification methods (e.g., PCR) or a device (e.g., a microarray). As is described above, the expression level of the biomarker of SEQ ID NO: 171 in the patient sample may then be compared, e.g., to the expression level of the biomarker of SEQ ID NO: 171 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 171 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-170 and 172-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 172 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 172 may be assessed using nucleic acid amplification methods (e.g., PCR) or a device (e.g., a microarray). As is described above, the expression level of the biomarker of SEQ ID NO: 172 in the patient sample may then be compared, e.g., to the expression level of the biomarker of SEQ ID NO: 172 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 172 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-171 and 173-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 173 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 173 may be assessed using nucleic acid amplification methods (e.g., PCR) or a device (e.g., a microarray). As is described above, the expression level of the biomarker of SEQ ID NO: 173 in the patient sample may then be compared, e.g., to the expression level of the biomarker of SEQ ID NO: 173 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 173 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-172 and 174-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 174 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 174 may be assessed using nucleic acid amplification methods (e.g., PCR) or a device (e.g., a microarray). As is described above, the expression level of the biomarker of SEQ ID NO: 174 in the patient sample may then be compared, e.g., to the expression level of the biomarker of SEQ ID NO: 174 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 174 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-173 and 175-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 175 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 175 may be assessed using nucleic acid amplification methods (e.g., PCR) or a device (e.g., a microarray). As is described above, the expression level of the biomarker of SEQ ID NO: 175 in the patient sample may then be compared, e.g., to the expression level of the biomarker of SEQ ID NO: 175 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 175 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-174 and 176-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 176 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 176 may be assessed using nucleic acid amplification methods (e.g., PCR) or a device (e.g., a microarray). As is described above, the expression level of the biomarker of SEQ ID NO: 176 in the patient sample may then be compared, e.g., to the expression level of the biomarker of SEQ ID NO: 176 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 176 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-175 and 177-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 177 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 177 may be assessed using nucleic acid amplification methods (e.g., PCR) or a device (e.g., a microarray). As is described above, the expression level of the biomarker of SEQ ID NO: 177 in the patient sample may then be compared, e.g., to the expression level of the biomarker of SEQ ID NO: 177 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 177 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-176 and 178-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 178 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 178 may be assessed using nucleic acid amplification methods (e.g., PCR) or a device (e.g., a microarray). As is described above, the expression level of the biomarker of SEQ ID NO: 178 in the patient sample may then be compared, e.g., to the expression level of the biomarker of SEQ ID NO: 178 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 178 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-177 and 179-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 179 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 179 may be assessed using nucleic acid amplification methods (e.g., PCR) or a device (e.g., a microarray). As is described above, the expression level of the biomarker of SEQ ID NO: 179 in the patient sample may then be compared, e.g., to the expression level of the biomarker of SEQ ID NO: 179 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 179 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-178 and 180-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 180 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 180 may be assessed using nucleic acid amplification methods (e.g., PCR) or a device (e.g., a microarray). As is described above, the expression level of the biomarker of SEQ ID NO: 180 in the patient sample may then be compared, e.g., to the expression level of the biomarker of SEQ ID NO: 180 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 180 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-179 and 181-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 181 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 181 may be assessed using nucleic acid amplification methods (e.g., PCR) or a device (e.g., a microarray). As is described above, the expression level of the biomarker of SEQ ID NO: 181 in the patient sample may then be compared, e.g., to the expression level of the biomarker of SEQ ID NO: 181 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 181 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-180 and 182-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 182 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 182 may be assessed using nucleic acid amplification methods (e.g., PCR) or a device (e.g., a microarray). As is described above, the expression level of the biomarker of SEQ ID NO: 182 in the patient sample may then be compared, e.g., to the expression level of the biomarker of SEQ ID NO: 182 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 182 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-181 and 183-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 183 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 183 may be assessed using nucleic acid amplification methods (e.g., PCR) or a device (e.g., a microarray). As is described above, the expression level of the biomarker of SEQ ID NO: 183 in the patient sample may then be compared, e.g., to the expression level of the biomarker of SEQ ID NO: 183 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 183 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-182 and 184-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 184 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 184 may be assessed using nucleic acid amplification methods (e.g., PCR) or a device (e.g., a microarray). As is described above, the expression level of the biomarker of SEQ ID NO: 184 in the patient sample may then be compared, e.g., to the expression level of the biomarker of SEQ ID NO: 184 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 184 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-183 and 185-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 185 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 185 may be assessed using nucleic acid amplification methods (e.g., PCR) or a device (e.g., a microarray). As is described above, the expression level of the biomarker of SEQ ID NO: 185 in the patient sample may then be compared, e.g., to the expression level of the biomarker of SEQ ID NO: 185 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 185 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-184 and 186-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 186 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 186 may be assessed using nucleic acid amplification methods (e.g., PCR) or a device (e.g., a microarray). As is described above, the expression level of the biomarker of SEQ ID NO: 186 in the patient sample may then be compared, e.g., to the expression level of the biomarker of SEQ ID NO: 186 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 186 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-185 and 187-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 187 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 187 may be assessed using nucleic acid amplification methods (e.g., PCR) or a device (e.g., a microarray). As is described above, the expression level of the biomarker of SEQ ID NO: 187 in the patient sample may then be compared, e.g., to the expression level of the biomarker of SEQ ID NO: 187 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 187 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-186 and 188-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 188 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 188 may be assessed using nucleic acid amplification methods (e.g., PCR) or a device (e.g., a microarray). As is described above, the expression level of the biomarker of SEQ ID NO: 188 in the patient sample may then be compared, e.g., to the expression level of the biomarker of SEQ ID NO: 188 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 188 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-187 and 189-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 189 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 189 may be assessed using nucleic acid amplification methods (e.g., PCR) or a device (e.g., a microarray). As is described above, the expression level of the biomarker of SEQ ID NO: 189 in the patient sample may then be compared, e.g., to the expression level of the biomarker of SEQ ID NO: 189 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 189 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-188 and 190-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 190 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 190 may be assessed using nucleic acid amplification methods (e.g., PCR) or a device (e.g., a microarray). As is described above, the expression level of the biomarker of SEQ ID NO: 190 in the patient sample may then be compared, e.g., to the expression level of the biomarker of SEQ ID NO: 190 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 190 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-189 and 191-193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 191 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 191 may be assessed using nucleic acid amplification methods (e.g., PCR) or a device (e.g., a microarray). As is described above, the expression level of the biomarker of SEQ ID NO: 191 in the patient sample may then be compared, e.g., to the expression level of the biomarker of SEQ ID NO: 191 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 191 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-190, 192, and 193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 192 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 192 may be assessed using nucleic acid amplification methods (e.g., PCR) or a device (e.g., a microarray). As is described above, the expression level of the biomarker of SEQ ID NO: 192 in the patient sample may then be compared, e.g., to the expression level of the biomarker of SEQ ID NO: 192 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 192 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-191, and 193 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

The biomarker of SEQ ID NO: 193 may be used to assess the responsiveness of a cancer patient (e.g., a patient with a cancer or a recurrence thereof) to ixabepilone or a pharmaceutically acceptable salt thereof. The expression level of the biomarker of SEQ ID NO: 193 may be assessed using nucleic acid amplification methods (e.g., PCR) or a device (e.g., a microarray). As is described above, the expression level of the biomarker of SEQ ID NO: 193 in the patient sample may then be compared, e.g., to the expression level of the biomarker of SEQ ID NO: 193 in a cell (e.g., a cancer cell) or tissue (e.g., a tumor tissue) known to be sensitive or resistant to treatment with ixabepilone or a pharmaceutically acceptable salt thereof and used to determine the cancer patient's responsiveness to ixabepilone or a pharmaceutically acceptable salt thereof. The biomarker of SEQ ID NO: 193 may be used alone or in combination with one or more additional biomarker(s) (e.g., one, two, three, four, five, ten, fifteen, twenty, twenty five, or all of the biomarkers shown in Tables 1 and/or 2 (e.g., the top one biomarker shown in Tables 1 and/or 2, the top two biomarker shown in Tables 1 and/or 2, the top three biomarkers shown in Tables 1 and/or 2, the top four biomarkers shown in Tables 1 and/or 2, the top five biomarkers shown in Tables 1 and/or 2, the top ten biomarkers shown in Tables 1 and/or 2, the top fifteen biomarkers shown in Tables 1 and/or 2, the top twenty biomarkers shown in Tables 1 and/or 2, the top twenty five biomarkers shown in Tables 1 and/or 2, or all of the biomarkers shown in Tables 1 and/or 2)), such as biomarker(s) of SEQ ID NOs: 1-192 to predict responsiveness of the cancer patient to treatment with ixabepilone or a pharmaceutically acceptable salt thereof. The expression level of the biomarker(s) may be determined using, e.g., a microarray, PCR, or other techniques described herein, for example, using a nucleic acid probe sequence based on the target sequences shown in Tables 1 and 2.

### Methods of Treatment

The diagnostic methods of the invention permit the assessment of whether a patient is likely to be responsive to treatment with ixabepilone or a pharmaceutically acceptable salt thereof and can thus be used to direct the patient's treatment (e.g., as a first line therapy and/or as a second or third line therapy). A patient to be treated or tested for responsiveness to ixabepilone or a pharmaceutically acceptable salt thereof according to the methods may include, e.g., a patient that has been diagnosed with cancer, a patient that has not received a cancer treatment (e.g., an anti-cancer agent other than ixabepilone or a pharmaceutically acceptable salt thereof, or radiation), a patient that has received a cancer treatment (e.g., an anti-cancer agent other than ixabepilone or radiation), or a patient during treatment with ixabepilone or a pharmaceutically acceptable salt thereof. For example, the patient may have a solid tumor or a hematological cancer, such as a cancer type selected from breast cancer (e.g., medullary carcinoma), myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, prostate cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), ovarian cancer (e.g., ovarian adenocarcinoma or embryonal carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), ER-positive cancer, bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), metastatic cancer, oral cavity cancer, uterine cancer, testicular cancer (e.g., seminoma and embryonal carcinoma), skin cancer (e.g., squamous cell carcinoma and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, liver cancer (e.g., hepatocellular carcinoma or hepatoma), larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, and neoplasms of the central nervous system. In particular, the cancer of the patient is, e.g., multiple myeloma, breast cancer, acute myelogenous leukemia (AML), acute lympho-blastic leukemia (ALL), chronic lymphocytic leukemia (CLL), myelodysplastic syndrome (MDS), chronic myelogenous leukemia-chronic phase (CMLCP), diffuse large B-cell lymphoma (DLBCL), cutaneous T-cell lymphoma (CTCL), peripheral T-cell lymphoma (PTCL), Hodgkin's lymphoma, hepatocellular carcinoma (HCC), cervical cancer, prostate cancer, kidney cancer, renal cell carcinoma (RCC), esophageal cancer, melanoma, glioma, pancreatic cancer, ovarian cancer, gastrointestinal stromal tumors (GIST), sarcoma, estrogen receptor-positive (ERpos) breast cancer, endometrial cancer, lung cancer, non-small cell lung carcinoma (NSCLC), mesothelioma, intestinal cancer, colon cancer, bladder cancer, adrenal cancer, gallbladder cancer, and squamous cell carcinoma of the head and neck (SCCHN). In particular, the patient may have estrogen receptor-positive (ER pos) breast cancer.

A patient found to be responsive to ixabepilone or a pharmaceutically acceptable salt thereof according to the methods of the invention may be preferentially selected for treatment with ixabepilone or a pharmaceutically acceptable salt thereof. For example, a patient can be identified as responsive to ixabepilone or a pharmaceutically acceptable salt thereof by determining the expression level of one or more biomarkers (e.g., one or more of the biomarkers shown in Table(s) 1 and/or 2, such as HLA-DRA (SEQ ID NO: 1) and/or PLK2 (SEQ ID NO: 47)) in a biological sample (e.g., a tumor sample) obtained from the patient, and subsequently administered ixabepilone or a pharmaceutically acceptable salt thereof. One or more additional therapies (e.g., surgery, radiation, or a therapeutic agent) may also be administered to the patient prior to, concurrently with, or after administration of ixabepilone or a pharmaceutically acceptable salt thereof. A therapeutic agent that can be administered to the patient prior to, concurrently with, or after administration of ixabepilone or a pharmaceutically acceptable salt thereof may be one or more of capecitabine, a histone deacetylase (HDAC) inhibitor, an immune checkpoint inhibitor, an antiestrogen, an aromatase inhibitor, an antigonadotropin, a proteasome inhibitor, an immunomodulator, a glucocorticoid, a folic acid, a monoclonal antibody, or an antineoplastic agent. In particular, a therapeutic agent that can be administered to the patient prior to, concurrently with, or after administration of ixabepilone or a pharmaceutically acceptable salt thereof may be one or more of capecitabine, an HDAC inhibitor, an immune checkpoint inhibitor (e.g., a PD1 inhibitor (e.g., Pembrolizumab (KEYTRUDA®), Nivolumab (OPDIVO®), and Cemiplimab (LIBTAYO®)), a PD-L1 inhibitor (e.g., Atezolizumab (TECENTRIQ®), Avelumab (BAVENCIO®), and Durvalumab (IMFINZI®)), and a CTLA-4 inhibitor (e.g., Ipilimumab (YERVOY®), and Tremelimumab)), an aromatase inhibitor (e.g., a non-selective aromatase inhibitor, such as Aminoglutethimide and Testolactone (TESLAC®), a selective aromatase inhibitor, such as anastrozole (ARIMIDEX®), letrozole (FEMARA®), exemestane (AROMASIN®), vorozole (RIVIZOR®), formestane (LENTARON®), and fadrozole (AFEMA®), and other aromatase inhibitors, such as 1,4,6-Androstatrien-3,17-dione (ATD) and 4-Androstene-3,6,17-trione (6-OXO)), an antiestrogen (e.g., a selective estrogen receptor modulator (SERM) (e.g., tamoxifen, clomifene, and raloxifene), an estrogen receptor silent antagonist, and selective estrogen receptor degrader (SERD) (e.g., fulvestrant (FASLODEX®))), an antigonadotropin (e.g., gonadotropin-releasing hormone (GnRH) analogue, compounds acting on sex steroid hormone receptors (e.g., progestogens, androgens, and estrogens), and steroid synthesis inhibitors (e.g., danazol and gestrinone)), a cyclin-dependent kinase inhibitor (e.g., a CDK inhibitor selective for CDK4 and CDK6, such as palbociclib (IBRANCE®) and abemaciclib (VERZENIO®, VERZENIOS®)), venetoclax (VENCLEXTA®, VENCLYXTO®), ibrutinib (IMBRUVICA®), bortezomib, carfilzomib, thalidomide, lenalidomide, pomalidomide, prednisone, dexamethasone, cyclophosphamide, vincristine, doxorubicin, melphalan, tegafur, irinotecan, oxaliplatin, cetuximab, leucovorin, SN-38, everolimus, temsirolimus, bleomycin, lomustine, depsipeptide, carboplatin, erlotinib, gemcitabine, mitoxantrone, cisplatin, busulfan, epirubicin, arsenic trioxide, bendamustine, teniposide, adriamycin, decitabine, estramustine, etoposide, azaguanine, aclarubicin, mitoxantrone, mitomycin, paclitaxel, taxotere, Irofulven, 5-FU, ara-c, methylprednisolone, methotrexate, methyl-gag, belinostat, carboplatin, idarubicin, IL4-PR38, valproic acid, all-trans retinoic acid (ATRA), cytoxan, topotecan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, floxuridine, thioguanine, PSC 833, herceptin, bevacizumab, celecoxib, iressa, anastrozole, letrozole, and rituximab. In a particular embodiment, the therapeutic agent that can be administered to the patient prior to, concurrently with, or after administration of ixabepilone or a pharmaceutically acceptable salt thereof is capecitabine. For example, capecitabine administration may be combined with administration of ixabepilone or a pharmaceutically acceptable salt thereof in a treatment regimen that includes administering ixabepilone or a pharmaceutically acceptable salt thereof to the subject as a three-hour infusion containing a dose of 40 mg/m² ixabepilone on day one of a three week treatment cycle and co-administering capecitabine (e.g., prior to, concurrently with, or after administration of ixabepilone or a pharmaceutically acceptable salt thereof) twice daily at a dose of 1000 mg/m² on days 1-14 of a three week treatment cycle. The number of treatment cycles (e.g., one, two, three, four, or more) can be determined based on measures of therapeutic efficacy of ixabepilone treatment.

Additionally or alternatively, an aromatase inhibitor (e.g., a non-selective aromatase inhibitor, such as Aminoglutethimide and Testolactone (TESLAC®), a selective aromatase inhibitor, such as anastrozole (ARIMIDEX®), letrozole (FEMARA®), exemestane (AROMASIN®), vorozole (RIVIZOR®), formestane (LENTARON®), and fadrozole (AFEMA®), and other aromatase inhibitors, such as 1,4,6-Androstatrien-3,17-dione (ATD) and 4-Androstene-3,6,17-trione (6-OXO)) may be administered to the patient prior to, concurrently with, or after administration of ixabepilone or a pharmaceutically acceptable salt thereof.

Additionally, or alternatively, an antiestrogen (e.g., a selective estrogen receptor modulator (SERM) (e.g., tamoxifen, clomifene, and raloxifene), an estrogen receptor silent antagonist, and selective estrogen receptor degrader (SERD) (e.g., fulvestrant (FASLODEX®))) may be administered to the patient prior to, concurrently with, or after administration of ixabepilone or a pharmaceutically acceptable salt thereof.

Additionally, or alternatively, an antigonadotropin (e.g., gonadotropin-releasing hormone (GnRH) analogue, compounds acting on sex steroid hormone receptors (e.g., progestogens, androgens, and estrogens), and steroid synthesis inhibitors (e.g., danazol (DANOCRINE®) and gestrinone (DIMETROSE®))) may be administered to the patient prior to, concurrently with, or after administration of ixabepilone or a pharmaceutically acceptable salt thereof.

The therapeutic agent (e.g., capecitabine) can be administered parenterally (e.g. intravenously, intramuscularly, transdermally, intradermally, intra-arterially, intracranially, subcutaneously, intraorbitally, intraventricularly, intraspinally, intraperitoneally, or intranasally), enterally, or topically.

Alternatively, a patient can be, e.g., identified as less likely to be responsive to ixabepilone or a pharmaceutically acceptable salt thereof by determining the expression level of one or more biomarkers (e.g., one or more of the biomarkers shown in Table(s) 1 and/or 2, such as HLA-DRA (SEQ ID NO: 1) and/or PLK2 (SEQ ID NO: 47)) in a biological sample obtained from the patient. If the patient exhibits expression levels of one or more biomarkers indicative of non-responsiveness to ixabepilone, the patient can be administered a treatment other than ixabepilone or a pharmaceutically acceptable salt thereof (e.g., surgery, radiation, or a therapeutic agent). In particular, the therapeutic agent may be one or more of capecitabine, a histone deacetylase (HDAC) inhibitor, an immune checkpoint inhibitor, ipilimumab, a cyclin-dependent kinase inhibitor (e.g., a CDK inhibitor selective for CDK4 and CDK6, such as palbociclib (IBRANCE®) and abemaciclib (VERZENIO®, VERZENIOS®)), venetoclax (VENCLEXTA®, VENCLYXTO®), ibrutinib (IMBRUVICA®), bortezomib, carfilzomib, thalidomide, lenalidomide, pomalidomide, prednisone, dexamethasone, cyclophosphamide, vincristine, doxorubicin, melphalan, capecitabine, tegafur, irinotecan, oxaliplatin, cetuximab, leucovorin, SN-38, everolimus, temsirolimus, bleomycin, lomustine, depsipeptide, carboplatin, erlotinib, gemcitabine, mitoxantrone, cisplatin, busulfan, epirubicin, arsenic trioxide, bendamustine, fulvestrant, teniposide, adriamycin, decitabine, estramustine, etoposide, azaguanine, aclarubicin, mitoxantrone, mitomycin, paclitaxel, taxotere, Irofulven, 5-FU, ara-c, methylprednisolone, methotrexate, methyl-gag, belinostat, carboplatin, idarubicin, IL4-PR38, valproic acid, all-trans retinoic acid (ATRA), cytoxan, topotecan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, tamoxifen, floxuridine, thioguanine, PSC 833, herceptin, bevacizumab, celecoxib, iressa, anastrozole, letrozole, and rituximab. The therapeutic agent can be administered parenterally (e.g. intravenously, intramuscularly, transdermally, intradermally, intra-arterially, intracranially, subcutaneously, intraorbitally, intraventricularly, intraspinally, intraperitoneally, or intranasally), enterally, or topically. In particular, the patient may be treated with, e.g., surgery, radiation, and/or administration of a therapeutic agent, such as, e.g., capecitabine.

Administration of ixabepilone Once a patient has been determined to be responsive to ixabepilone or a pharmaceutically acceptable salt thereof, according to the methods described herein, ixabepilone or a pharmaceutically acceptable salt thereof may be administered to the patient, for example, parenterally (e.g., infusion or injection), enterally, or topically. Parenteral routes of ixabepilone (e.g., ixabepilone or a pharmaceutically acceptable salt thereof) administration include intravenous (e.g., infusion or injection), transdermal, intradermal, intramuscular, intra-arterial, intracranial, subcutaneous, intraorbital, intraventricular, intraspinal, intraperitoneal, or intranasal. Enteral routes of ixabepilone (e.g., ixabepilone or a pharmaceutically acceptable salt thereof) administration may include oral, buccal, sublabial, sublingual, or by inhalation. The preferred route for administration of ixabepilone may be via intravenous infusion.

The effective amount of ixabepilone or a pharmaceutically acceptable salt thereof to be administered to a subject may be determined by one of ordinary skill in the art, and includes exemplary dosage amounts for a human from about 0.05 to 200 mg/kg/day (e.g., 0.05-200 mg/kg/day, 0.1-175 mg/kg/day, 0.2-150 mg/kg/day, 0.3-150 mg/kg/day, 0.4-125 mg/kg/day, 0.5-100 mg/kg/day, 1-75 mg/kg/day, 5-50 mg/kg/day, 10-40 mg/kg/day, 15-30 mg/kg/day, and 20-25 mg/kg/day), which may be administered in a single dose or in the form of individual divided doses, such as from one to four times per day. In particular embodiments, ixabepilone or a pharmaceutically acceptable salt thereof is administered at a dosage of less than 100 mg/kg/day in a single dose or in two to four divided doses. In other embodiments, ixabepilone or a pharmaceutically acceptable salt thereof is administered at, e.g., a dose of about 3-12000 mg, 5-10000 mg, 10-7500 mg, 20-5000 mg, 25-2500 mg, 30-2000 mg, 35-1500 mg, 40-1250 mg, 45-1000 mg, 50-750 mg, 55-500 mg, 60-250 mg, 65-100 mg, 70-90 mg, 75-85 mg, and 80-85 mg. In particular, ixabepilone or a pharmaceutically acceptable salt thereof may be administered at doses of about 10 mg, 20 mg, 30 mg, 40 mg, 50 mg, 80 mg, 100 mg, 200 mg, 250 mg, 500 mg, 600 mg, or 750 mg. In other embodiments, ixabepilone or a pharmaceutically acceptable salt thereof is administered at, e.g., a dose of about 1-200 mg/m² (e.g., 1-200 mg/m², 5-150 mg/m², 10-100 mg/m², 15-75 mg/m², 20-50 mg/m², 25-50 mg/m², 30-45 mg/m², and 40-45 mg/m²,). In preferred embodiments, ixabepilone or a pharmaceutically acceptable salt thereof is be administered at a dose of about 40 mg/m². For example, about 40 mg/m² of ixabepilone or a pharmaceutically acceptable salt thereof can be administered by way of intravenous infusion over a period of 3 hours. In other preferred embodiments, ixabepilone or a pharmaceutically acceptable salt thereof is be administered at a dose of about 80 mg. For example, about 80 mg of ixabepilone or a pharmaceutically acceptable salt thereof can be administered by way of intravenous infusion over a period of 3 hours. Ixabepilone or a pharmaceutically acceptable salt thereof may be administered to the patient one or more times, such as one or more times hourly, daily, weekly, every two weeks, every three weeks (e.g., administered on day one of a three week treatment cycle), every four weeks, monthly, every two months, every three months, every six months, or every year. Preferably, ixabepilone may be administered once every three weeks (e.g., on day one of a three-week treatment cycle). For example, ixabepilone or a pharmaceutically acceptable salt thereof can be administered via intravenous infusion at a dose of about 40 mg/m² or at a dose of about 80 mg once daily (e.g., ixabepilone or a pharmaceutically acceptable salt thereof can be administered via intravenous infusion prepared by making a constituted solution having ixabepilone or a pharmaceutically acceptable salt thereof at a concentration of 2 mg/mL and subsequently diluting the constituted solution with one of three specified infusion fluids to a final concentration of 0.2 mg/mL to 0.6 mg/mL). The infusion fluids that may be used for diluting the constituted solution containing ixabepilone or a pharmaceutically acceptable salt thereof include Lactated Ringer's Injection, USP (United States Pharmacopeia). Alternatively, the infusion fluid may be a 0.9% Sodium Chloride Injection, USP (pH adjusted with a Sodium Bicarbonate Injection, USP). For example, a 250 mL or a 500 mL bag of 0.9% Sodium Chloride Injection may be used to prepare the infusion. The pH may be adjusted to values between 6.0 and 9.0 by adding 2 mEg (e.g., 2 mL of an 8.4% w/v solution or 4 mL of a 4.2% w/v solution) of Sodium Bicarbonate Injection, prior to addition of the constituted solution containing ixabepilone or a pharmaceutically acceptable salt thereof. As another alternative, the infusion fluid may be PLASMA-LYTE A Injection pH 7.4.

The method may further include administering a second dose of ixabepilone or a pharmaceutically acceptable salt thereof to the patient two days, four days, six days, one week, two weeks, three weeks (e.g., on day one of a three week treatment cycle), four weeks, or five weeks after administration of a first dose of ixabepilone or a pharmaceutically acceptable salt thereof. The administration of ixabepilone or a pharmaceutically acceptable salt thereof can be repeated at such a frequency for a certain period of time, followed by a period without treatment. Such repeated administrations can occur over a course of therapy lasting a specified length of time (e.g., at least 1 week, 2 weeks, 3 weeks, 1 month, 2 months, 3 months, 6 months, 8 months, 10 months, 12 months, 18 months, 24 months, 36 months, 48 months, or 60 months).

The patient may be administered a pharmaceutically acceptable salt of ixabepilone. Pharmaceutically acceptable salts of ixabepilone described herein may include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of ixabepilone described herein or separately by reacting a free base group with a suitable organic acid.

Ixabepilone may have ionizable groups so as to be capable of preparation as pharmaceutically acceptable salts. These salts may be acid addition salts involving inorganic or organic acids or the salts may, in the case of acidic forms of ixabepilone be prepared from inorganic or organic bases. Frequently, ixabepilone may be prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

Ixabepilone or a pharmaceutically acceptable salt thereof can be administered in a pharmaceutical composition that includes one or more pharmaceutically acceptable carriers, excipients, or diluents. Examples of suitable carriers, excipients, or diluents of ixabepilone or a pharmaceutically acceptable salt thereof include, e.g., saline, sterile water, polyalkylene glycols, oils of vegetable origin, hydrogenated napthalenes, suitable buffer, 1,3-butanediol, Ringer's solution and/or sodium chloride solution. Exemplary formulations for parenteral administration can include solutions prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Other exemplary carriers, excipients, or diluents are described in the Handbook of Pharmaceutical Excipients, 6th Edition, Rowe et al., Eds., Pharmaceutical Press (2009), hereby incorporated by reference in its entirety.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). In all cases, the composition must be sterile and should be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating ixabepilone or a pharmaceutically acceptable salt thereof in the required amount in an appropriate solvent with one or a combination of ingredients enumerated above, as required, followed by filtered sterilization. Dispersions can be prepared by incorporating ixabepilone or a pharmaceutically acceptable salt thereof into a sterile vehicle which contains a basic dispersion medium and the required other ingredients from those enumerated above. In the case of sterile powders for the preparation of sterile injectable solutions, the preferred methods of preparation can be vacuum drying and freeze-drying which yields a powder of ixabepilone or a pharmaceutically acceptable salt thereof plus any additional desired ingredient from a previously sterile-filtered solution thereof.

Oral compositions can include an inert diluent or an edible carrier. The composition can be enclosed in a gelatin capsule or compressed into a tablet. For the purpose of oral therapeutic administration, ixabepilone or a pharmaceutically acceptable salt thereof can be incorporated with excipients and used in the form of tablets, troches, or gelatin capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated can be used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, ixabepilone or a pharmaceutically acceptable salt thereof may be formulated into ointments, salves, gels, or creams as generally known in the art.

Ixabepilone or a pharmaceutically acceptable salt thereof can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art.

The pharmaceutical compositions can be included in a container, pack, or dispenser together with instructions for administration.

Methods of formulating pharmaceutical agents are known in the art, e.g., Niazi, Handbook of Pharmaceutical Manufacturing Formulations (Second Edition), CRC Press 2009, describes formulation development for liquid, sterile, compressed, semi-compressed and OTC forms. Transdermal and mucosal delivery, lymphatic system delivery, nanoparticles, controlled drug release systems, theranostics, protein and peptide drugs, and biologics delivery are described in Wang et al., Drug Delivery: Principles and Applications (Second Edition), Wiley 2016; formulation and delivery of peptide and protein agent is described, e.g., in Banga, Therapeutic Peptides and Proteins: Formulation, Processing, and Delivery Systems (Third Edition), CRC Press 2015.

### Kits

Kits of the invention can be used for determining the responsiveness of a cancer patient (e.g., a solid tumor, such as breast cancer) to ixabepilone or a pharmaceutically acceptable salt thereof. Kits of the invention can include reagents and/or materials for, e.g., collecting and/or purifying nucleic acids from biological samples (such as those obtained from a patient to be treated with ixabepilone or a pharmaceutically acceptable salt thereof), reagents for amplifying such nucleic acids to produce an amplified sample, and/or at least one device of the invention. Reagents for amplifying nucleic acids may include, e.g., PCR reagents, including but not limited to DNA polymerase, RNA polymerase, PCR buffer, magnesium chloride solutions, nucleic acid primers (e.g., primers designed to target particular biomarkers of responsiveness to ixabepilone or a pharmaceutically acceptable salt thereof), and/or any other PCR reagents as are well known in the art. In particular, kits useful in the method may include includes one or more of the following: a kit for RNA extraction from tumors (e.g., Trizol for mRNA, mirVana miRNA isolation kit from Ambion Inc), a kit for RNA labeling (e.g., MessageAmp from Ambion Inc., FlashTag from Genisphere Inc), a microarray for measuring biomarker expression (e.g., HG-U133A, HG-U133_Plus2 or miRNA-1.0 from Affymetrix Inc), a microarray hybridization station and scanner (e.g., GeneChip System 3000Dx from Affymetrix Inc), and/or software for analyzing the expression of biomarker genes or RNAs (e.g., miRNAs) as described in herein (e.g., implemented in R from R-Project or S-Plus from Insightful Corp.).

For example, a kit of the invention can include one or more probes capable of detecting one or more biomarkers of Table(s) 1 and/or 2 (e.g., the kit may include probes for the biomarkers of Tables 1 and 2). Such probes can, for example, include nucleic acids capable of hybridizing to the biomarker based on nucleic acid sequence complementarity. In particular, a probe has at least 85% sequence identity (e.g., 85%, 90%, 95%, 97%, 98%, 99%, or 100% sequence identity) to a nucleic acid sequence that is complementary or identical to at least 5 (e.g., at least 15) consecutive nucleotides of one or more biomarkers. The probes can be attached a solid surface, such as a microarray. The kit may include NanoString capture probes, NanoString reporter probes, and/or one or more nCounter cartridges. The kit may include reagents for next generation sequencing, including but not limited to poly(T) oligonucleotides, dye terminators, sequencing adapters, adapter ligation reagents, reverse transcriptase, primers (e.g., random primers), DNA-cleaving enzymes, polymerases, and/or any combination thereof. The kit may also be one that includes a protein array and/or reagents for detection of the polypeptide product(s) of one or more biomarkers of Table(s) 1 and/or 2.

### EXAMPLES

### Example 1. Identification of Biomarkers of Sensitivity and Resistance to Ixabepilone

Methodology of the In Vitro Cancer Growth Inhibition Screen

DNA chip measurements of the 60 cancer cell lines of the NC160 data set were performed using Affymetrix HG-U133Plus2 arrays and logit normalized. Specifically, the human tumor cell lines of the cancer screening panel were grown in RPMI 1640 medium containing 5% fetal bovine serum and 2 mM L-glutamine. Cells were inoculated into 96 well microtiter plates in 100 μL at plating densities ranging from 5,000 to 40,000 cells/well depending on the doubling time of individual cell lines. After cell inoculation, the microtiter plates were incubated at 37° C., 5% C02, 95% air and 100% relative humidity for 24 hours prior to addition of ixabepilone.

After 24 hours, two plates of each cell line were fixed in situ with TCA, to represent a measurement of the cell population for each cell line at the time of compound addition (Tz). Ixabepilone was solubilized in dimethyl sulfoxide at 400-fold the desired final maximum test concentration and stored frozen prior to use. At the time ixabepilone addition, an aliquot of frozen concentrate was thawed and diluted to twice the desired final maximum test concentration with complete medium containing 50 μg/ml Gentamicin. Additional four, 10-fold or ½ log serial dilutions were made to provide a total of five concentrations plus control. Aliquots of 100 μl of these different compound dilutions were added to the appropriate microtiter wells already containing 100 μl of medium, resulting in the required final compound concentrations.

Following ixabepilone addition, the plates were incubated for an additional 48 h at 37° C., 5% C02, 95% air, and 100% relative humidity. For adherent cells, the assay were terminated by the addition of cold TCA. Cells were fixed in situ by the gentle addition of 50 μl of cold 50% (w/v) TCA (final concentration, 10% TCA) and incubated for 60 minutes at 4° C. The supernatant was discarded, and the plates were washed five times with tap water and air-dried. Sulforhodamine B (SRB) solution (100 μl) at 0.4% (w/v) in 1% acetic acid was added to each well, and plates were incubated for 10 minutes at room temperature. After staining, unbound dye was removed by washing five times with 1% acetic acid and the plates were air-dried. Bound stain was subsequently solubilized with 10 mM trizma base, and the absorbance was read on an automated plate reader at a wavelength of 515 nm. For suspension cells, the methodology was the same, except that the assay was terminated by fixing settled cells at the bottom of the wells by gently adding 50 μl of 80% TCA (final concentration, 16% TCA). Using the seven absorbance measurements (time zero, (Tz), control growth, (C), and test growth in the presence of compound (e.g., ixabepilone or a pharmaceutically acceptable salt thereof) at the five concentration levels (Ti)), the percentage growth was calculated at each of the ixabepilone concentrations levels. Percentage growth inhibition was calculated as:

[(Ti−Tz)/(C−Tz)]×100 for concentrations for which Ti>/=Tz

[(Ti−Tz)/Tz]×100 for concentrations for which Ti<Tz

Three dose response parameters were calculated for each ixabepilone. Growth inhibition of 50% (GI50) was calculated from [(Ti−Tz)/(C−Tz)]×100=50, which is the ixabepilone concentration resulting in a 50% reduction in the net protein increase (as measured by SRB staining) in control cells during the compound incubation. The compound concentration resulting in total growth inhibition (TGI) was calculated from Ti=Tz. The LC50 (concentration of compound resulting in a 50% reduction in the measured protein at the end of the compound treatment as compared to that at the beginning) indicating a net loss of cells following treatment was calculated from [(Ti−Tz)/Tz]×100=−50. Values were calculated for each of these three parameters if the level of activity was reached; however, if the effect was not reached or was exceeded, the value for that parameter was expressed as greater or less than the maximum or minimum concentration tested.

Gene Expression and Growth Inhibition Analysis

The gene expression measurements of NC160 cancer cell lines were obtained from a publicly available database (e.g., the National Cancer Institute and the Massachusetts Institute of Technology). Each dataset was normalized so that sample expression measured by different chips could be compared. The preferred method of normalization is the logit transformation, which was performed for each gene y on each chip. For each array, the logit transformation was performed as follows:

logit(y)=log [(y−background)/(saturation−y)],

where background was calculated as the minimum intensity measured on the chip minus 0.1% of the signal intensity range: min−0.001*(max−min), and saturation was calculated as the maximum intensity measured on the chip plus 0.1% of the signal intensity range: max+0.001*(max−min). The resulting logit transformed data was then z-transformed to mean zero and standard deviation 1.

Gene expression was then correlated to cancer cell growth inhibition (−log(GI50)) in the presence of ixabepilone. Growth inhibition data of ixabepilone against the same cell lines were downloaded from the National Cancer Institute. The expression level of each of the genes of Tables 1 and 2 in each cell line was correlated to the growth of those cell lines (log(GI50)) in the presence of ixabepilone. The Pearson correlation coefficient was then determined to identify genes positively and negatively correlated to sensitivity to ixabepilone. Tables 1 and 2 show the top positively correlated genes (the biomarkers of sensitivity) and negatively correlated genes (the biomarkers of resistance). In particular, genes with a Pearson correlation greater than 0.25 or below −0.25 were classified as biomarkers of sensitivity or resistance, respectively.

Spearman Rank-Order correlation coefficient may also be used in computing the correlation coefficient between gene expression and cancer cell growth inhibition. Instead of using GI50s, any other measure of patient sensitivity to a given treatment (e.g., ixabepilone or a pharmaceutically acceptable salt thereof) may also be correlated to a gene expression levels of the patient. Since a plurality of measurements may be available for a single gene, the most accurate determination of correlation coefficient can be, e.g., the median of the correlation coefficients calculated for all probes measuring expression of the same gene. Although the present example employed a correlation coefficient cutoff of 0.25, the present methods may also be used in conjunction with other cutoff values. For example, the median correlation coefficient of gene expression measured on a probe to growth inhibition or patient sensitivity to ixabepilone or a pharmaceutically acceptable salt thereof can be calculated for all genes of interest. Genes that have a median correlation above, e.g., 0.25, 0.30, 0.31, 0.32, 0.33, 0.34, 0.35, 0.36, 0.37, 0.38, 0.39, 0.40, or higher, can be used as biomarkers of sensitivity for assessing responsiveness of a cancer patient (e.g., a patient have recurrence of cancer) to ixabepilone or a pharmaceutically acceptable salt thereof. Likewise, genes that have a median correlation below, e.g., −0.25, −0.30, −0.31, −0.32, −0.33, −0.34, −0.35, −0.36, −0.37, −0.38, −0.39, −0.40, or lower, can be used as biomarkers of resistance for assessing responsiveness of a cancer patient (e.g., a patient with a recurrence of cancer) to ixabepilone or a pharmaceutically acceptable salt thereof.

### Example 2. Predicting Responsiveness of a Breast Cancer Patient to Ixabepilone

An mRNA-based predictor of responsiveness to ixabepilone was developed according to the methods of the invention and applied to gene expression data prepared using clinical samples from breast cancer patients, which was downloaded from Gene Expression Omnibus under accession number GSE41998 (FIG. 1). The samples were diagnostic biopsies analyzed with Affymetrix GeneChips. All the genes listed in Table 2 were measured from clinical samples on the Affymetrix GeneChip and their mean expression subtracted from the mean of all the genes in Table 1. The difference (e.g., a difference score) was then compared to a difference score produced using measured levels of of the sensitivity and resistance biomarkers of Tables 1 and 2 from a reference population consisting of 279 breast cancer patients participating in a clinical trial having two treatment arms ((1) ixabepilone and (2) paclitaxel treatment), producing a percentile score on a scale from 0 to 100. As part of the clinical trial, biopsy samples from breast cancer patients later treated neoadjuvantly with ixabepilone were collected prior to treatment. Patients' responses to ixabepilone were subsequently compared to predicted ixabepilone sensitivity using the methods disclosed herein. Patients with pathological complete remission (pCR) were predicted using the disclosed methods to be more sensitive to ixabepilone than patients with no pCR (No_pCR; FIG. 1). Table 3 shows the number of responders correctly predicted at a cutoff of 50, demonstrating an increased response rate for breast cancer patients having DRP scores above the cutoff.

TABLE 3
Number of predicted responders at a cutoff of 50

| | | Non |
|---|---|---|
| | Responders | responders |
| | (pCR) | (no pCR) |
| DRP positive (>50) | 27 | 45 |
| DRP negative (<=50) | 8 | 52 |

Overall percent agreement between DRP and clinic: (27 + 52)//27 + 52 + 8 + 45) = 60%
Sensitivity: 27/(27 + 8) = 77% of responders correctly predicted
Specificity: 52/(45 + 52) = 54% of non-responders correctly predicted

One possible way to compare the clinical performance of different biomarkers using a single parameter for comparison is by measuring the Pearson correlation between the clinical outcome (e.g., pCR=1, no pCR=0) and prediction score for the biomarker. As is shown in Table 4 below, the Pearson correlation coefficient values were obtained for the entire panel of biomarkers of sensitivity of Table 1 and biomarkers of resistance of Table 2 as well as for subsets of biomarkers from each of Tables 1 and 2. While the highest correlation between clinical outcome and biomarker prediction score was obtained using the full panel of biomarkers of Tables 1 and 2, comparable correlation values correlation were observed using only the top gene of sensitivity of Table 1 (HLA-DRA) or the top 15 genes of Table 1 (i.e., HLA-DRA, HLA-DRA, ICAM3, ITGB7, CD8B, CD74, HLA-DRB1 HLA-DRB4 HLA-DRB5 LOC100507709 LOC100507714, IGJ, HLA-DRB1 HLA-DRB3 HLA-DRB4 LOC100507709 LOC100507714, HLA-DPA1, HLA-DRB1 LOC100507709 LOC100507714, HLA-DPA1, CD28, CD37, and NHP2) and the top gene of resistance of Table 2 (i.e., PLK2) or the top 15 genes of Table 2 (i.e., PLK2, PLXNB2, RRAS2, RRAS2, PTPLA, P2RX5-TAX1BP3 TAX1 BP3, STAT3, PPIC, PTRF, RCN1, ZFP36L1, GFPT1, ACTN1, SEPT10, and ACTN1). Therefore, responsiveness of breast cancer patients to ixabepilone can be successfully assessed using the full panel of the biomarkers of sensitivity of Table 1 and biomarkers of resistance of Table 2 or using a small subset of biomarkers of Tables 1 and 2 (e.g., 1 biomarker of sensitivity and 1 biomarker of resistance or, e.g., two or more, or all, of the top 15 biomarkers of sensitivity and two or more, or all, of the top 15 biomarkers of resistance) that show the highest absolute correlation values between the clinical outcome and the biomarker prediction score.

TABLE 4
Clinical performance of different biomarkers.

| | Pearson |
|---|---|
| Biomarker | correlation |
| 46 genes in Table 1-144 genes in Table 2 | 0.294 |
| 15 top genes in Table 1-15 top genes in Table 2 | 0.288 |
| HLA-DRA-PLK2 | 0.287 |

### Example 3. Predicting Treatment Response Rate as a Function of DRP Cutoff

As shown in FIG. 2, the response rate depends on the cutoff applied to the DRP scores demonstrated in FIG. 1; the higher the cutoff, the higher the predicted likelihood of and the rate of response to the treatment. Using FIG. 2 as a guide, it is evident that the ixabepilone DRP biomarker can substantially increase the response rate in a selected subset of patients in any cancer type. For example, Table 3 illustrates that using a cutoff of 50 substantially improves the response rate among breast cancer patients treated with ixabepilone. Other cutoff values may also be used in conjunction with the methods disclosed herein.

### Example 4. Treating a Cancer Patient with Ixabepilone

Cancer patients (e.g., those diagnosed with breast cancer) can be treated with ixabepilone or a pharmaceutically acceptable salt thereof after being predicted to be responsive to treatment with ixabepilone or a pharmaceutically acceptable salt thereof by the methods described herein. In particular, the cancer patient may be one that has not previously received any cancer treatment or one that has received a cancer treatment other than ixabepilone or a pharmaceutically acceptable salt thereof. Moreover, the patient may be one diagnosed with a cancer (e.g., breast cancer) or with recurrence thereof.

The cancer patient can be determined to be responsive to ixabepilone or a pharmaceutically acceptable salt thereof following the methods disclosed herein. Once the patient has been determined to be responsive to ixabepilone or a pharmaceutically acceptable salt thereof, according to the methods described herein, ixabepilone or a pharmaceutically acceptable salt thereof may be administered to the patient, for example, by way of intravenous infusion over a period of three hours as a daily dose of 40 mg/m² (e.g., the patient can be administered ixabepilone on day one of a three week treatment cycle). Ixabepilone or a pharmaceutically acceptable salt thereof may be administered to the patient alone or in combination with an additional therapy, such as a capecitabine, which can be administered prior to, concurrently with, or after administration of ixabepilone or the pharmaceutically acceptable salt thereof. For example, the cancer patient can be administered ixabepilone as a 40 mg/m² 3-hour infusion on day one and capecitabine 1000 mg/m² twice daily on days 1-14 of a three week treatment cycle.

Ixabepilone or a pharmaceutically acceptable salt thereof may be administered to the patient one time during the first treatment regimen (e.g., ixabepilone administered on day one of a first three-week treatment cycle). The patient can be administered a second treatment regimen of ixabepilone or a pharmaceutically acceptable salt thereof three weeks after administration of the first treatment regimen of ixabepilone or a pharmaceutically acceptable salt thereof. The administration of ixabepilone or a pharmaceutically acceptable salt thereof can be repeated at such a frequency for a certain period of time, followed by a period without treatment. Such repeated administrations can occur over a course of therapy lasting a specified length of time (e.g., at least 1 week, 2 weeks, 3 weeks, 1 month, 2 months, 3 months, 6 months, or 1 year or more).

### Example 5. Predicting Responsiveness of a Breast Cancer Patient to Ixabepilone Using a Panel of Biomarkers of Sensitivity and Biomarkers of Resistance to Ixabepilone

The diagnostic methods described herein can be used to predict the responsiveness of a breast cancer patient to treatment with ixabepilone or a pharmaceutically acceptable salt thereof. In particular, the breast cancer patient may be one that has not previously received any cancer treatment or one that has received a cancer treatment other than ixabepilone or a pharmaceutically acceptable salt thereof. Moreover, the patient may be one diagnosed with breast cancer or with recurrence of breast cancer.

A biological sample (e.g., a breast tissue sample, such as a breast tissue sample obtained by biopsy) may be obtained from the patient through methods well known in the art. The sample may be frozen and/or prepared, e.g., by formalin fixation and paraffin embedding. In particular, mRNA can be isolated from the sample and a gene expression profile can be determined, e.g., using a microarray platform, such as the Affymetrix HG-U133A, for one or more of the biomarkers shown in Table(s) 1 and/or 2. One or more of the biomarkers shown in Table(s) 1 and/or 2 can also be measured, e.g., by sequencing or PCR-based techniques, such as those described herein.

For example, the expression level of one or more biomarkers of sensitivity to ixabepilone or a pharmaceutically acceptable salt thereof can be determined in the sample from the patient, such as one or more biomarkers of SEQ ID NO: 1-46. The expression level of one or more biomarkers of resistance to ixabepilone can also be determined in the sample from the patient, such as one or more of SEQ ID NO: 47-193. The patient's responsiveness to ixabepilone or a pharmaceutically acceptable salt thereof can also be assessed by calculating a difference score for the patient (mean of expression of the biomarkers of sensitivity noted above minus the mean of expression of the biomarkers of resistance noted above).

The breast cancer patient would be determined to be responsive to ixabepilone or a pharmaceutically acceptable salt thereof when the expression level of the one or more biomarkers of sensitivity is similar to (e.g., substantially similar to) the expression level of the biomarkers of sensitivity in a cell or tissue (e.g., tumor tissue) known to be sensitive to ixabepilone or a pharmaceutically acceptable salt thereof. The breast cancer patient would also be determined to be responsive to ixabepilone or a pharmaceutically acceptable salt thereof when the expression level of one or more of the biomarkers of resistance is similar to (e.g., substantially similar to) the expression level of the biomarkers of resistance in a cell or tissue (e.g., a tumor tissue) known to be sensitive to ixabepilone or a pharmaceutically acceptable salt thereof. The breast cancer patient would also be determined to be responsive to ixabepilone or a pharmaceutically acceptable salt thereof when difference score for the patient is similar to (e.g., substantially similar to) the difference score in a cell or tissue (e.g., a tumor tissue) known to be sensitive to ixabepilone or a pharmaceutically acceptable salt thereof (e.g., the difference score of a reference subject having the same diagnosis as the patient that is already determined to be responsive to ixabepilone or a pharmaceutically acceptable salt thereof).

Alternatively, the breast cancer patient may be determined to be responsive to ixabepilone or a pharmaceutically acceptable salt thereof if the expression level of one or more of the biomarkers of sensitivity is dissimilar to (e.g., substantially dissimilar to) the expression level of the biomarkers of sensitivity in a cell or tissue (e.g., a tumor tissue) known to be resistant to ixabepilone or a pharmaceutically acceptable salt thereof. The breast cancer patient may also be responsive to ixabepilone or a pharmaceutically acceptable salt thereof if the expression level of one or more of the biomarkers of resistance is dissimilar to (e.g., substantially dissimilar to) the expression level of the biomarkers of resistance in a cell or tissue (e.g., a tumor tissue) known to be resistant to ixabepilone or a pharmaceutically acceptable salt thereof. The breast cancer patient would also be determined to be responsive to ixabepilone or a pharmaceutically acceptable salt thereof when difference score for the patient is dissimilar to (e.g., substantially dissimilar to) the difference score in a cell or tissue (e.g., a tumor tissue) known to be resistant to ixabepilone or a pharmaceutically acceptable salt thereof (e.g., the difference score of a reference subject having the same diagnosis as the patient that is already determined to be resistant to ixabepilone or a pharmaceutically acceptable salt thereof).

If the patient is predicted to be responsive, then the patient can be administered ixabepilone or a pharmaceutically acceptable salt thereof, such as ixabepilone or a pharmaceutically acceptable salt thereof administered as an intravenous infusion at a dose of about 40 mg/m². Conversely, if the patient is predicted to be non-responsive to ixabepilone (e.g., ixabepilone or a pharmaceutically acceptable salt thereof) treatment, then the patient can be administered one or more therapies other than ixabepilone or a pharmaceutically acceptable salt thereof, such as radiation or a therapeutic agent (e.g., capecitabine, and/or another therapeutic agent described herein). For example, the patient can be administered about 40 mg/m² of ixabepilone or a pharmaceutically acceptable salt thereof by way of intravenous infusion over a period of 3 hours on day one of a three week treatment cycle and, optionally, the patient may additionally receive about 1000 mg mg/m² of capecitabine twice daily on days 1-14 of a three week treatment cycle.

### Example 5. Predicting Responsiveness of Breast Cancer Patients to Ixabepilone Using a Single Biomarker of Sensitivity and a Single Biomarker of Resistance to Ixabepilone

The diagnostic methods described herein can be used to predict the responsiveness of a breast cancer patient to treatment with ixabepilone or a pharmaceutically acceptable salt thereof. In particular, the breast cancer patient may be one that has not previously received any cancer treatment or one that has received a cancer treatment other than ixabepilone or a pharmaceutically acceptable salt thereof. Moreover, the patient may be one diagnosed with breast cancer or with recurrence of breast cancer.

A biological sample (e.g., a breast tissue sample, such as a breast tissue sample obtained by biopsy) may be obtained from the patient through methods well known in the art. The sample may be frozen and/or prepared, e.g., by formalin fixation and paraffin embedding. In particular, mRNA can be isolated from the sample and a gene expression profile can be determined, e.g., using a microarray platform, such as the Affymetrix HG-U133A, for one or more of the biomarkers shown in Table(s) 1 and/or 2. One or more of the biomarkers shown in Table(s) 1 and/or 2 can also be measured, e.g., by sequencing or PCR-based techniques, such as those described herein.

In particular, the expression level of a single biomarker of sensitivity to ixabepilone or a pharmaceutically acceptable salt thereof can be determined in the sample from the patient, such as, e.g., the biomarker HLA-DRA (SEQ ID NO: 1). The expression level of a single biomarker of resistance to ixabepilone can also be determined in the sample from the patient, such as, e.g., the biomarker PLK2 (SEQ ID NO: 47). The patient's responsiveness to ixabepilone or a pharmaceutically acceptable salt thereof can also be assessed by calculating a difference score for the patient (mean of expression of HLA-DRA (SEQ ID NO: 1) noted above minus the mean of expression of the PLK2 (SEQ ID NO: 47) noted above).

The breast cancer patient would be determined to be responsive to ixabepilone or a pharmaceutically acceptable salt thereof when the expression level of HLA-DRA (SEQ ID NO: 1) is similar to (e.g., substantially similar to) the expression level of HLA-DRA (SEQ ID NO: 1) in a cell or tissue (e.g., tumor tissue) known to be sensitive to ixabepilone or a pharmaceutically acceptable salt thereof. The breast cancer patient would also be determined to be responsive to ixabepilone or a pharmaceutically acceptable salt thereof when the expression level of PLK2 (SEQ ID NO: 47) is similar to (e.g., substantially similar to) the expression level of PLK2 (SEQ ID NO: 47) in a cell or tissue (e.g., a tumor tissue) known to be sensitive to ixabepilone or a pharmaceutically acceptable salt thereof. The breast cancer patient would also be determined to be responsive to ixabepilone or a pharmaceutically acceptable salt thereof when difference score for the patient is similar to (e.g., substantially similar to) the difference score in a cell or tissue (e.g., a tumor tissue) known to be sensitive to ixabepilone or a pharmaceutically acceptable salt thereof (e.g., the difference score of a reference subject having the same diagnosis as the patient that is already determined to be responsive to ixabepilone or a pharmaceutically acceptable salt thereof).

Alternatively, the breast cancer patient may be determined to be responsive to ixabepilone or a pharmaceutically acceptable salt thereof if the expression level of HLA-DRA (SEQ ID NO: 1) is dissimilar to (e.g., substantially dissimilar to) the expression level of HLA-DRA (SEQ ID NO: 1) in a cell or tissue (e.g., a tumor tissue) known to be resistant to ixabepilone or a pharmaceutically acceptable salt thereof. The breast cancer patient may also be responsive to ixabepilone or a pharmaceutically acceptable salt thereof if the expression level of PLK2 (SEQ ID NO: 47) is dissimilar to (e.g., substantially dissimilar to) the expression level of PLK2 (SEQ ID NO: 47) in a cell or tissue (e.g., a tumor tissue) known to be resistant to ixabepilone or a pharmaceutically acceptable salt thereof. The breast cancer patient would also be determined to be responsive to ixabepilone or a pharmaceutically acceptable salt thereof when difference score for the patient is dissimilar to (e.g., substantially dissimilar to) the difference score in a cell or tissue (e.g., a tumor tissue) known to be resistant to ixabepilone or a pharmaceutically acceptable salt thereof (e.g., the difference score of a reference subject having the same diagnosis as the patient that is already determined to be resistant to ixabepilone or a pharmaceutically acceptable salt thereof).

If the patient is predicted to be responsive, then the patient can be administered ixabepilone or a pharmaceutically acceptable salt thereof, such as ixabepilone or a pharmaceutically acceptable salt thereof administered as an intravenous infusion at a dose of about 40 mg/m². Conversely, if the patient is predicted to be non-responsive to ixabepilone (e.g., ixabepilone or a pharmaceutically acceptable salt thereof) treatment, then the patient can be administered one or more therapies other than ixabepilone or a pharmaceutically acceptable salt thereof, such as radiation or a therapeutic agent (e.g., capecitabine, and/or another therapeutic agent described herein). For example, the patient can be administered about 40 mg/m² of ixabepilone or a pharmaceutically acceptable salt thereof by way of intravenous infusion over a period of 3 hours on day one of a three week treatment cycle and, optionally, the patient may additionally receive about 1000 mg mg/m² of capecitabine twice daily on days 1-14 of a three week treatment cycle.

### Other Embodiments

All publications, patents, and patent applications mentioned in the above specification are hereby incorporated by reference. Various modifications and variations of the described device and methods of use of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. For example, it is anticipated that measuring the level of proteins, metabolites, identifying genetic mutations and DNA copy number variations, all will be useful in determining patient responsiveness.

Some embodiments of the invention described herein can be defined according to any one of the following enumerated paragraphs:
E1. A method of determining responsiveness of a subject with a cancer to ixabepilone or a pharmaceutically acceptable salt thereof including:
   (a) contacting a sample from the subject including one or more nucleic acid molecules with a device including:
      (i) one or more single-stranded nucleic acid molecules capable of specifically hybridizing with the nucleotides of one or more biomarkers of sensitivity selected from the biomarkers of Table 1; and/or
      (ii) one or more single-stranded nucleic acid molecules capable of specifically hybridizing with the nucleotides of one or more biomarkers of resistance selected from the biomarkers of Table 2; and
   (b) measuring hybridization between the one or more nucleic acid molecules from the sample and the single-stranded nucleic acid molecules of the device to detect a level of expression of the one or more biomarkers of sensitivity and/or the one or more biomarkers of resistance.
E2. The method of E1, wherein the subject is determined to be responsive to ixabepilone or a pharmaceutically acceptable salt thereof if:
   (i) the level of expression of the one or more biomarkers of sensitivity is substantially similar to the level of expression of the one or more biomarkers of sensitivity in a cell or tissue known to be sensitive to ixabepilone or a pharmaceutically acceptable salt thereof;
   (ii) the level of expression of the one or more biomarkers of resistance is substantially similar to the level of expression of the one or more biomarkers of resistance in a cell or tissue known to be sensitive to ixabepilone or a pharmaceutically acceptable salt thereof;
   (iii) the level of expression of the one or more biomarkers of sensitivity is substantially dissimilar to the level of expression of the one or more biomarkers of sensitivity in a cell or tissue known to be resistant to ixabepilone or a pharmaceutically acceptable salt thereof; and/or
   (iv) the level of expression of the one or more biomarkers of resistance is substantially dissimilar to the level of expression of the one or more biomarkers of resistance in a cell or tissue known to be resistant to ixabepilone or a pharmaceutically acceptable salt thereof.
E3. The method of E1, further including administering ixabepilone or a pharmaceutically acceptable salt thereof to the subject.
E4. The method of E3, wherein ixabepilone or the pharmaceutically acceptable salt thereof is administered to the subject:
   a) at a dose of 40 mg/m²;
   b) at a dose of about 40-120 mg; or
   c) at a dose of 80 mg.
E5. The method of E3 or E4, wherein the ixabepilone or the pharmaceutically acceptable salt thereof is administered to the subject by parenteral administration.
E6. The method of E5, wherein the parenteral administration includes intravenous infusion or injection.
E7. The method of any one of E3-E6, including:
   a) administering ixabepilone or the pharmaceutically acceptable salt thereof to the subject two or more times;
   b) administering ixabepilone or the pharmaceutically acceptable salt thereof to the subject one or more times daily, weekly, every two weeks, every three weeks, or monthly.
E8. The method of E7, including administering ixabepilone or the pharmaceutically acceptable salt thereof to the subject one or more times every three weeks.
E9. The method of E8, wherein the intravenous infusion is performed over a period of three hours on day one of a three-week treatment cycle.
E10. The method of any one of E3-E9, wherein the ixabepilone or the pharmaceutically acceptable salt thereof is formulated for administration as a solution including ixabepilone at a concentration of 0.2 mg/mL to 0.6 mg/mL.
E11. The method of any one of E1-E10, further including administering one or more cancer therapies other than ixabepilone or a pharmaceutically acceptable salt thereof to the subject, wherein, optionally, the one or more cancer therapies includes surgery, radiation, or a therapeutic agent.
E12. The method of E11, wherein the therapeutic agent is selected from the group consisting of capecitabine, a histone deacetylase (HDAC) inhibitor, an immune checkpoint inhibitor, a cyclin-dependent kinase (CDK) inhibitor, bortezomib, carfilzomib, thalidomide, lenalidomide, pomalidomide, prednisone, dexamethasone, cyclophosphamide, vincristine, doxorubicin, melphalan, tegafur, irinotecan, oxaliplatin, cetuximab, leucovorin, SN-38, everolimus, temsirolimus, bleomycin, lomustine, depsipeptide, carboplatin, erlotinib, gemcitabine, mitoxantrone, cisplatin, busulfan, epirubicin, arsenic trioxide, bendamustine, fulvestrant, teniposide, adriamycin, decitabine, estramustine, etoposide, azaguanine, aclarubicin, mitoxantrone, mitomycin, paclitaxel, taxotere, Irofulven, 5-FU, ara-c, methylprednisolone, methotrexate, methyl-gag, belinostat, carboplatin, idarubicin, IL4-PR38, valproic acid, all-trans retinoic acid (ATRA), cytoxan, topotecan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, tamoxifen, floxuridine, thioguanine, PSC 833, herceptin, bevacizumab, celecoxib, iressa, anastrozole, letrozole, and rituximab.
E13. The method of E12, wherein the therapeutic agent is capecitabine.
E14. The method of E12 or E13, including:
   a) administering capecitabine to the subject two or more times;
   b) administering capecitabine to the subject one or more times daily, weekly, every two weeks, every three weeks, or monthly.
E15. The method of E14, including administering capecitabine to the subject one or more times every three weeks.
E16. The method of E14 or E15, wherein the capecitabine is administered to the subject two times per day on days 1-14 of a three-week treatment cycle.
E17. The method of any one of E12-E16, wherein the capecitabine is administered to the subject:
   a) at a dose of 1000 mg/m²;
   b) at a dose of about 2000-2500 mg; or
   c) at a dose of 2000 mg.
E18. A method of treating a cancer in a subject in need thereof including administering ixabepilone or a pharmaceutically acceptable salt thereof to the subject, wherein the subject has been determined to be responsive to ixabepilone or the pharmaceutically acceptable salt thereof according to the method of E1.
E19. A method of treating a subject with a cancer, including:
   (a) contacting a sample from the subject including one or more nucleic acid molecules with a device including:
      (i) one or more single-stranded nucleic acid molecules capable of specifically hybridizing with the nucleotides of one or more biomarkers of sensitivity selected from the biomarkers of Table 1; and/or
      (ii) one or more single-stranded nucleic acid molecules capable of specifically hybridizing with the nucleotides of one or more biomarkers of resistance selected from the biomarkers of Table 2;
   (b) measuring hybridization between the one or more nucleic acid molecules from the sample and the single-stranded nucleic acid molecules of the device to detect a level of expression of the one or more biomarkers of sensitivity and/or the one or more biomarkers of resistance; and
   (c) administering ixabepilone or a pharmaceutically acceptable salt thereof to the subject.
E20. The method of E19, wherein the subject is administered ixabepilone or the pharmaceutically acceptable salt thereof if:
   (i) the level of expression of the one or more biomarkers of sensitivity is substantially similar to the level of expression of the one or more biomarkers of sensitivity in a cell or tissue known to be sensitive to ixabepilone or a pharmaceutically acceptable salt thereof;
   (ii) the level of expression of the one or more biomarkers of resistance is substantially similar to the level of expression of the one or more biomarkers of resistance in a cell or tissue known to be sensitive to ixabepilone or a pharmaceutically acceptable salt thereof;
   (iii) the level of expression of the one or more biomarkers of sensitivity is substantially dissimilar to the level of expression of the one or more biomarkers of sensitivity in a cell or tissue known to be resistant to ixabepilone or a pharmaceutically acceptable salt thereof; and/or
   (iv) the level of expression of the one or more biomarkers of resistance is substantially dissimilar to the level of expression of the one or more biomarkers of resistance in a cell or tissue known to be resistant to ixabepilone or a pharmaceutically acceptable salt thereof.
E21. The method of any one of E18-E20, wherein ixabepilone or the pharmaceutically acceptable salt thereof is administered to the subject:
   a) at a dose of 40 mg/m²;
   b) at a dose of about 40-120 mg; or
   c) at a dose of 80 mg.
E22. The method of any one of E18-E21, wherein the ixabepilone or the pharmaceutically acceptable salt thereof is administered to the subject by way of parenteral administration.
E23. The method of E22, wherein the parenteral administration includes intravenous infusion or injection.
E24. The method of any one of E18-E23, including:
   a) administering ixabepilone or the pharmaceutically acceptable salt thereof to the subject two or more times;
   b) administering ixabepilone or the pharmaceutically acceptable salt thereof to the subject one or more times daily, weekly, every two weeks, every three weeks, or monthly.
E25. The method of E24, including administering ixabepilone or the pharmaceutically acceptable salt thereof to the subject one or more times every three weeks.
E26. The method of any one of E23-E25, wherein the intravenous infusion is performed over a period of three hours on day one of a three-week treatment cycle.
E27. The method of any one of E18-E26, wherein the ixabepilone or the pharmaceutically acceptable salt thereof is formulated as a solution including ixabepilone at a concentration of 0.2 mg/mL to 0.6 mg/mL.
E28. The method of any one of E18-E27, further including administering one or more additional therapies to the subject prior to, concurrently with, or after administration of ixabepilone or the pharmaceutically acceptable salt thereof, wherein, optionally, the one or more additional therapies includes surgery, radiation, or a therapeutic agent.
E29. The method of E28, wherein the therapeutic agent is selected from the group consisting of capecitabine, a histone deacetylase (HDAC) inhibitor, an immune checkpoint inhibitor, an antiestrogen, an aromatase inhibitor, an antigonadotropin, a proteasome inhibitor, an immunomodulator, a glucocorticoid, a folic acid, a monoclonal antibody, and an antineoplastic agent, wherein optionally the therapeutic agent is fulvestrant, ipilimumab, a cyclin-dependent kinase (CDK) inhibitor, bortezomib, carfilzomib, thalidomide, lenalidomide, pomalidomide, prednisone, dexamethasone, cyclophosphamide, vincristine, doxorubicin, melphalan, tegafur, irinotecan, oxaliplatin, cetuximab, leucovorin, SN-38, everolimus, temsirolimus, bleomycin, lomustine, depsipeptide, carboplatin, erlotinib, gemcitabine, mitoxantrone, cisplatin, busulfan, epirubicin, arsenic trioxide, bendamustine, teniposide, adriamycin, decitabine, estramustine, etoposide, azaguanine, aclarubicin, mitoxantrone, mitomycin, paclitaxel, taxotere, Irofulven, 5-FU, ara-c, methylprednisolone, methotrexate, methyl-gag, belinostat, carboplatin, idarubicin, IL4-PR38, valproic acid, all-trans retinoic acid (ATRA), cytoxan, topotecan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, tamoxifen, clomifene, raloxifene, floxuridine, thioguanine, PSC 833, herceptin, bevacizumab, celecoxib, iressa, anastrozole, letrozole, or rituximab.
E30. The method of E29, wherein the therapeutic agent is capecitabine.
E31. The method of E29 or E30, including:
   a) administering capecitabine to the subject two or more times;
   b) administering capecitabine to the subject one or more times daily, weekly, every two weeks, every three weeks, or monthly.
E32. The method of E31, including administering capecitabine to the subject one or more times every three weeks.
E33. The method of E32, wherein the capecitabine is administered to the subject two times per day on days 1-14 of a three-week treatment cycle.
E34. The method of any one of E29-E33, wherein the capecitabine is administered to the subject:
   a) at a dose of 1000 mg/m²;
   b) at a dose of about 2000-2500 mg; or
   c) at a dose of 2000 mg.
E35. The method of any one of E1-E34, wherein the device is a microarray, wherein, optionally, the microarray is a deoxyribonucleic acid (DNA)-based platform.
E36. The method of any one of E1-E35, wherein:
   a) the device includes at least two, at least three, at least four, at least five, at least six, at least seven, at least eight, at least nine, at least ten, or more single-stranded nucleic acid molecules of (i) and/or (ii);
   b) the one or more single-stranded nucleic acid molecules of the device have a length in the range of 10-100 nucleotides;
   c) the method includes converting the level of expression of the one or more biomarkers of sensitivity and/or the one or more biomarkers of resistance into a mean score, wherein the mean score indicates the responsiveness of the subject to ixabepilone or a pharmaceutically acceptable salt thereof;
   d) the level of expression of the one or more biomarkers of sensitivity and/or the one or more biomarkers of resistance is determined by microarray analysis or nucleic acid amplification methods.
E37. The method of E36, further including subtracting the mean score for the one or more biomarkers of resistance from the mean score for the one or more biomarkers of sensitivity to obtain a difference score, wherein the difference score indicates the responsiveness of the subject to ixabepilone or a pharmaceutically acceptable salt thereof.
E38. The method of E36, wherein the mean score and/or the difference score above a cutoff value indicates that the subject is responsive to ixabepilone or a pharmaceutically acceptable salt thereof.
E39. The method of E38, wherein the cutoff value is established as the 50^{th} percentile, or 60th percentile, or 70^{th} percentile, or 80^{th} percentile, or 90^{th} percentile or greater in a reference population, such as a sample(s) from a tumor of the same type as that of the subject.
E40. The method of E38 or E39, wherein the cutoff value is established as the 50^{th} percentile in a reference population, such as a sample(s) from a tumor of the same type as that of the subject.
E41. The method of any one of E1-E40, wherein:
   (i) the level of expression of the biomarkers of sensitivity is determined by detecting the level of mRNA transcribed from a gene coding one or more of the biomarkers of Table 1; and/or
   (ii) the level of expression of the biomarkers of resistance is determined by detecting the level of mRNA transcribed from a gene coding one or more of the biomarkers of Table 2.
E42. The method of any one of E1-E40, wherein:
   a)
      (i) the biomarkers of sensitivity are selected from at least 1, at least 5, at least 10, at least 15, at least 20, or at least 25 of the biomarkers of Table 1; and/or
      (ii) the biomarkers of resistance are selected from at least 1, at least 5, at least 10, at least 15, at least 20, or at least 25 of the biomarkers of Table 2;
   b)
      (i) the biomarker of sensitivity is HLA-DRA (SEQ ID NO: 1); and/or
      (ii) the biomarker of resistance is PLK2 (SEQ ID NO: 47).
E43. The method of any one of E1-E42, wherein the cancer is selected from a solid tumor cancer.
E44. The method of E43, wherein the solid tumor cancer is breast cancer.
E45. The method of any one of E1-E44, wherein the subject has recurrence of cancer.
E46. The method of any one of E1-E45, wherein the sample from the subject is a tumor sample.

What is claimed is:

1. A method of determining responsiveness of a subject with a cancer to ixabepilone or a pharmaceutically acceptable salt thereof comprising:
(a) contacting a sample from the subject comprising one or more nucleic acid molecules with a device comprising:
(i) one or more single-stranded nucleic acid molecules capable of specifically hybridizing with the nucleotides of one or more biomarkers of sensitivity selected from the biomarkers of Table 1; and/or
(ii) one or more single-stranded nucleic acid molecules capable of specifically hybridizing with the nucleotides of one or more biomarkers of resistance selected from the biomarkers of Table 2; and
(b) measuring hybridization between the one or more nucleic acid molecules from the sample and the single-stranded nucleic acid molecules of the device to detect a level of expression of the one or more biomarkers of sensitivity and/or the one or more biomarkers of resistance.

2. The method of claim 1, wherein the subject is determined to be responsive to ixabepilone or a pharmaceutically acceptable salt thereof if:
(i) the level of expression of the one or more biomarkers of sensitivity is substantially similar to the level of expression of the one or more biomarkers of sensitivity in a cell or tissue known to be sensitive to ixabepilone or a pharmaceutically acceptable salt thereof;
(ii) the level of expression of the one or more biomarkers of resistance is substantially similar to the level of expression of the one or more biomarkers of resistance in a cell or tissue known to be sensitive to ixabepilone or a pharmaceutically acceptable salt thereof;
(iii) the level of expression of the one or more biomarkers of sensitivity is substantially dissimilar to the level of expression of the one or more biomarkers of sensitivity in a cell or tissue known to be resistant to ixabepilone or a pharmaceutically acceptable salt thereof; and/or
(iv) the level of expression of the one or more biomarkers of resistance is substantially dissimilar to the level of expression of the one or more biomarkers of resistance in a cell or tissue known to be resistant to ixabepilone or a pharmaceutically acceptable salt thereof.

3. The method of claim 1, further comprising administering ixabepilone or a pharmaceutically acceptable salt thereof to the subject.

4. The method of claim 3, wherein ixabepilone or the pharmaceutically acceptable salt thereof is administered to the subject:
a) at a dose of 40 mg/m²;
b) at a dose of about 40-120 mg; or
c) at a dose of 80 mg.

5. The method of claim 3, wherein the ixabepilone or the pharmaceutically acceptable salt thereof is administered to the subject by parenteral administration.

6. The method of claim 5, wherein the parenteral administration comprises intravenous infusion or injection.

7. The method of claim 3, comprising:
a) administering ixabepilone or the pharmaceutically acceptable salt thereof to the subject two or more times;
b) administering ixabepilone or the pharmaceutically acceptable salt thereof to the subject one or more times daily, weekly, every two weeks, every three weeks, or monthly.

8. The method of claim 7, comprising administering ixabepilone or the pharmaceutically acceptable salt thereof to the subject one or more times every three weeks.

9. The method of claim 6, wherein the intravenous infusion is performed over a period of three hours on day one of a three-week treatment cycle.

10. The method of claim 3, wherein the ixabepilone or the pharmaceutically acceptable salt thereof is formulated for administration as a solution comprising ixabepilone at a concentration of 0.2 mg/mL to 0.6 mg/mL.

11. The method of claim 3, further comprising administering one or more cancer therapies other than ixabepilone or a pharmaceutically acceptable salt thereof to the subject, wherein, optionally, the one or more cancer therapies comprises surgery, radiation, or a therapeutic agent.

12. The method of claim 11, wherein the therapeutic agent is selected from the group consisting of capecitabine, a histone deacetylase (HDAC) inhibitor, an immune checkpoint inhibitor, a cyclin-dependent kinase (CDK) inhibitor, bortezomib, carfilzomib, thalidomide, lenalidomide, pomalidomide, prednisone, dexamethasone, cyclophosphamide, vincristine, doxorubicin, melphalan, tegafur, irinotecan, oxaliplatin, cetuximab, leucovorin, SN-38, everolimus, temsirolimus, bleomycin, lomustine, depsipeptide, carboplatin, erlotinib, gemcitabine, mitoxantrone, cisplatin, busulfan, epirubicin, arsenic trioxide, bendamustine, fulvestrant, teniposide, adriamycin, decitabine, estramustine, etoposide, azaguanine, aclarubicin, mitoxantrone, mitomycin, paclitaxel, taxotere, Irofulven, 5-FU, ara-c, methylprednisolone, methotrexate, methyl-gag, belinostat, carboplatin, idarubicin, IL4-PR38, valproic acid, all-trans retinoic acid (ATRA), cytoxan, topotecan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, tamoxifen, floxuridine, thioguanine, PSC 833, herceptin, bevacizumab, celecoxib, iressa, anastrozole, letrozole, and rituximab.

13. The method of claim 12, wherein the therapeutic agent is capecitabine.

14. The method of claim 12, comprising:
a) administering capecitabine to the subject two or more times;
b) administering capecitabine to the subject one or more times daily, weekly, every two weeks, every three weeks, or monthly.

15. The method of claim 14, comprising administering capecitabine to the subject one or more times every three weeks.

16. The method of claim 14, wherein the capecitabine is administered to the subject two times per day on days 1-14 of a three-week treatment cycle.

17. The method of claim 12, wherein the capecitabine is administered to the subject:
a) at a dose of 1000 mg/m²;
b) at a dose of about 2000-2500 mg; or
c) at a dose of 2000 mg;

18. A method of treating a cancer in a subject in need thereof comprising administering ixabepilone or a pharmaceutically acceptable salt thereof to the subject, wherein the subject has been determined to be responsive to ixabepilone or the pharmaceutically acceptable salt thereof according to the method of claim 1.

19. A method of treating a subject with a cancer, comprising:
(a) contacting a sample from the subject comprising one or more nucleic acid molecules with a device comprising:
(i) one or more single-stranded nucleic acid molecules capable of specifically hybridizing with the nucleotides of one or more biomarkers of sensitivity selected from the biomarkers of Table 1; and/or
(ii) one or more single-stranded nucleic acid molecules capable of specifically hybridizing with the nucleotides of one or more biomarkers of resistance selected from the biomarkers of Table 2;
(b) measuring hybridization between the one or more nucleic acid molecules from the sample and the single-stranded nucleic acid molecules of the device to detect a level of expression of the one or more biomarkers of sensitivity and/or the one or more biomarkers of resistance; and
(c) administering ixabepilone or a pharmaceutically acceptable salt thereof to the subject.

20. The method of claim 19, wherein the subject is administered ixabepilone or the pharmaceutically acceptable salt thereof if:
(i) the level of expression of the one or more biomarkers of sensitivity is substantially similar to the level of expression of the one or more biomarkers of sensitivity in a cell or tissue known to be sensitive to ixabepilone or a pharmaceutically acceptable salt thereof;
(ii) the level of expression of the one or more biomarkers of resistance is substantially similar to the level of expression of the one or more biomarkers of resistance in a cell or tissue known to be sensitive to ixabepilone or a pharmaceutically acceptable salt thereof;
(iii) the level of expression of the one or more biomarkers of sensitivity is substantially dissimilar to the level of expression of the one or more biomarkers of sensitivity in a cell or tissue known to be resistant to ixabepilone or a pharmaceutically acceptable salt thereof; and/or
(iv) the level of expression of the one or more biomarkers of resistance is substantially dissimilar to the level of expression of the one or more biomarkers of resistance in a cell or tissue known to be resistant to ixabepilone or a pharmaceutically acceptable salt thereof.

21. The method of 19, wherein ixabepilone or the pharmaceutically acceptable salt thereof is administered to the subject:
a) at a dose of 40 mg/m²;
b) at a dose of about 40-120 mg; or
c) at a dose of 80 mg.

22. The method of claim 19, wherein the ixabepilone or the pharmaceutically acceptable salt thereof is administered to the subject by way of parenteral administration.

23. The method of claim 22, wherein the parenteral administration comprises intravenous infusion or injection.

24. The method of claim 19, comprising:
a) administering ixabepilone or the pharmaceutically acceptable salt thereof to the subject two or more times;
b) administering ixabepilone or the pharmaceutically acceptable salt thereof to the subject one or more times daily, weekly, every two weeks, every three weeks, or monthly.

25. The method of claim 24, comprising administering ixabepilone or the pharmaceutically acceptable salt thereof to the subject one or more times every three weeks.

26. The method of claim 23, wherein the intravenous infusion is performed over a period of three hours on day one of a three-week treatment cycle.

27. The method of claim 19, wherein the ixabepilone or the pharmaceutically acceptable salt thereof is formulated as a solution comprising ixabepilone at a concentration of 0.2 mg/mL to 0.6 mg/mL.

28. The method of claim 19, further comprising administering one or more additional therapies to the subject prior to, concurrently with, or after administration of ixabepilone or the pharmaceutically acceptable salt thereof, wherein, optionally, the one or more additional therapies comprises surgery, radiation, or a therapeutic agent.

29. The method of claim 28, wherein the therapeutic agent is selected from the group consisting of capecitabine, HDAC inhibitor, an immune checkpoint inhibitor, an antiestrogen, an aromatase inhibitor, an antigonadotropin, a proteasome inhibitor, an immunomodulator, a glucocorticoid, a folic acid, a monoclonal antibody, and an antineoplastic agent, wherein optionally the therapeutic agent is fulvestrant, ipilimumab, CDK inhibitor, bortezomib, carfilzomib, thalidomide, lenalidomide, pomalidomide, prednisone, dexamethasone, cyclophosphamide, vincristine, doxorubicin, melphalan, tegafur, irinotecan, oxaliplatin, cetuximab, leucovorin, SN-38, everolimus, temsirolimus, bleomycin, lomustine, depsipeptide, carboplatin, erlotinib, gemcitabine, mitoxantrone, cisplatin, busulfan, epirubicin, arsenic trioxide, bendamustine, teniposide, adriamycin, decitabine, estramustine, etoposide, azaguanine, aclarubicin, mitoxantrone, mitomycin, paclitaxel, taxotere, Irofulven, 5-FU, ara-c, methylprednisolone, methotrexate, methyl-gag, belinostat, carboplatin, idarubicin, IL4-PR38, valproic acid, ATRA, cytoxan, topotecan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, tamoxifen, clomifene, raloxifene, floxuridine, thioguanine, PSC 833, herceptin, bevacizumab, celecoxib, iressa, anastrozole, letrozole, or rituximab.

30. The method of claim 29, wherein the therapeutic agent is capecitabine.

31. The method of claim 29, comprising:
a) administering capecitabine to the subject two or more times;
b) administering capecitabine to the subject one or more times daily, weekly, every two weeks, every three weeks, or monthly.

32. The method of claim 31, comprising administering capecitabine to the subject one or more times every three weeks.

33. The method of claim 32, wherein the capecitabine is administered to the subject two times per day on days 1-14 of a three-week treatment cycle.

34. The method of claim 29, wherein the capecitabine is administered to the subject:
a) at a dose of 1000 mg/m²;
b) at a dose of about 2000-2500 mg; or
c) at a dose of 2000 mg;

35. The method of claim 1, wherein the device is a microarray, wherein, optionally, the microarray is a deoxyribonucleic acid (DNA)-based platform.

36. The method of claim 1, wherein:
a) the device comprises at least two, at least three, at least four, at least five, at least six, at least seven, at least eight, at least nine, at least ten, or more single-stranded nucleic acid molecules of (i) and/or (ii);
b) the one or more single-stranded nucleic acid molecules of the device have a length in the range of 10-100 nucleotides;
c) the method comprises converting the level of expression of the one or more biomarkers of sensitivity and/or the one or more biomarkers of resistance into a mean score, wherein the mean score indicates the responsiveness of the subject to ixabepilone or a pharmaceutically acceptable salt thereof;
d) the level of expression of the one or more biomarkers of sensitivity and/or the one or more biomarkers of resistance is determined by microarray analysis or nucleic acid amplification methods.

37. The method of claim 36, further comprising subtracting the mean score for the one or more biomarkers of resistance from the mean score for the one or more biomarkers of sensitivity to obtain a difference score, wherein the difference score indicates the responsiveness of the subject to ixabepilone or a pharmaceutically acceptable salt thereof.

38. The method of claim 36, wherein the mean score and/or the difference score above a cutoff value indicates that the subject is responsive to ixabepilone or a pharmaceutically acceptable salt thereof.

39. The method of claim 38, wherein the cutoff value is established as the 50^{th} percentile, or 60th percentile, or 70^{th} percentile, or 80^{th} percentile, or 90^{th} percentile or greater in a reference population, such as a sample(s) from a tumor of the same type as that of the subject.

40. The method of claim 38, wherein the cutoff value is established as the 50^{th} percentile in a reference population, such as a sample(s) from a tumor of the same type as that of the subject.

41. The method of claim 1, wherein:
(i) the level of expression of the biomarkers of sensitivity is determined by detecting the level of mRNA transcribed from a gene coding one or more of the biomarkers of Table 1; and/or
(ii) the level of expression of the biomarkers of resistance is determined by detecting the level of mRNA transcribed from a gene coding one or more of the biomarkers of Table 2.

42. The method of claim 1, wherein:
a)
(i) the biomarkers of sensitivity are selected from at least 1, at least 5, at least 10, at least 15, at least 20, or at least 25 of the biomarkers of Table 1; and/or
(ii) the biomarkers of resistance are selected from at least 1, at least 5, at least 10, at least 15, at least 20, or at least 25 of the biomarkers of Table 2;
b)
(i) the biomarker of sensitivity is HLA-DRA (SEQ ID NO: 1); and/or
(ii) the biomarker of resistance is PLK2 (SEQ ID NO: 47).

43. The method of claim 1, wherein the cancer is selected from a solid tumor cancer.

44. The method of claim 43, wherein the solid tumor cancer is breast cancer.

45. The method of claim 1, wherein the subject has recurrence of cancer.

46. The method of claim 1, wherein the sample from the subject is a tumor sample.


SEQUENCE LISTING (193 sequences)

SEQ ID NO: 1 - DNA, 25 bp, Artificial Sequence
  Synthetic Construct
cgatcaccaatgtacctccagaggt

SEQ ID NO: 2 - DNA, 25 bp, Artificial Sequence
  Synthetic Construct
tgggagtttgatgctccaagccctc

SEQ ID NO: 3 - DNA, 25 bp, Artificial Sequence
  Synthetic Construct
accaacggagcggcagttaccatgt

SEQ ID NO: 4 - DNA, 25 bp, Artificial Sequence
  Synthetic Construct
tatgaccgccgggaatacagtcgct

SEQ ID NO: 5 - DNA, 25 bp, Artificial Sequence
  Synthetic Construct
ggtctttcatggggtgatgctgggc

SEQ ID NO: 6 - DNA, 25 bp, Artificial Sequence
  Synthetic Construct
acacaaggctccaagacctaggctc

SEQ ID NO: 7 - DNA, 25 bp, Artificial Sequence
  Synthetic Construct
gagtggtttctatccaggcagcatt

SEQ ID NO: 8 - DNA, 25 bp, Artificial Sequence
  Synthetic Construct
gtatcaaaatcttccaattatcatg

SEQ ID NO: 9 - DNA, 25 bp, Artificial Sequence
  Synthetic Construct
tctgtgagtggtttctatccaggca

SEQ ID NO: 10 - DNA, 25 bp, Artificial Sequence
  Synthetic Construct
ccagatgcctgagacaacggagact

SEQ ID NO: 11 - DNA, 25 bp, Artificial Sequence
  Synthetic Construct
accttccagaccctggtgatgctgg

SEQ ID NO: 12 - DNA, 25 bp, Artificial Sequence
  Synthetic Construct
tcattgagccttttatcctctgttc

SEQ ID NO: 13 - DNA, 25 bp, Artificial Sequence
  Synthetic Construct
gatgaatcacacttgagatgtttct

SEQ ID NO: 14 - DNA, 25 bp, Artificial Sequence
  Synthetic Construct
agctcagcaggcttggacacctggc

SEQ ID NO: 15 - DNA, 25 bp, Artificial Sequence
  Synthetic Construct
gaaatttgccctatgtctatatccc

SEQ ID NO: 16 - DNA, 25 bp, Artificial Sequence
  Synthetic Construct
gggggcctgctcagagaaggtgtca

SEQ ID NO: 17 - DNA, 25 bp, Artificial Sequence
  Synthetic Construct
aggctcctcacaggcgctgtgaatt

SEQ ID NO: 18 - DNA, 25 bp, Artificial Sequence
  Synthetic Construct
gctaaaacggagcttttttgccctg

SEQ ID NO: 19 - DNA, 25 bp, Artificial Sequence
  Synthetic Construct
ggagcaatccaagccacatatcttc

SEQ ID NO: 20 - DNA, 25 bp, Artificial Sequence
  Synthetic Construct
atttctaaccctgcaacttttgcca

SEQ ID NO: 21 - DNA, 25 bp, Artificial Sequence
  Synthetic Construct
tcgcccaccaaccaagtgttacaag

SEQ ID NO: 22 - DNA, 25 bp, Artificial Sequence
  Synthetic Construct
gctactcagagcaacaggttccaca

SEQ ID NO: 23 - DNA, 25 bp, Artificial Sequence
  Synthetic Construct
gttgtgttatagtttatgcttctta

SEQ ID NO: 24 - DNA, 25 bp, Artificial Sequence
  Synthetic Construct
tgaacaatcccggtgtgtcaggacc

SEQ ID NO: 25 - DNA, 25 bp, Artificial Sequence
  Synthetic Construct
actgtgcactccatcatcagcaaaa

SEQ ID NO: 26 - DNA, 25 bp, Artificial Sequence
  Synthetic Construct
actgtgatataggtactctgattta

SEQ ID NO: 27 - DNA, 25 bp, Artificial Sequence
  Synthetic Construct
gtcaccacgcggttctggaaaggca

SEQ ID NO: 28 - DNA, 25 bp, Artificial Sequence
  Synthetic Construct
tattgggtcaagctgcttacctgcc

SEQ ID NO: 29 - DNA, 25 bp, Artificial Sequence
  Synthetic Construct
cctgcttcgaaatgcctatcgggag

SEQ ID NO: 30 - DNA, 25 bp, Artificial Sequence
  Synthetic Construct
gagtgactagagctcactgtctact

SEQ ID NO: 31 - DNA, 25 bp, Artificial Sequence
  Synthetic Construct
catgtatacctggatttgcttggct

SEQ ID NO: 32 - DNA, 25 bp, Artificial Sequence
  Synthetic Construct
tatgaagcactttctcttaacttaa

SEQ ID NO: 33 - DNA, 25 bp, Artificial Sequence
  Synthetic Construct
gatgtatgtcgctgtccaagagaag

SEQ ID NO: 34 - DNA, 25 bp, Artificial Sequence
  Synthetic Construct
tgtgccaatcaatagcacccctact

SEQ ID NO: 35 - DNA, 25 bp, Artificial Sequence
  Synthetic Construct
gaaaggaggaaagccctctccaggc

SEQ ID NO: 36 - DNA, 25 bp, Artificial Sequence
  Synthetic Construct
gatcctatggtttctccatccaatt

SEQ ID NO: 37 - DNA, 25 bp, Artificial Sequence
  Synthetic Construct
gtctcactaggcatgctgttgtaca

SEQ ID NO: 38 - DNA, 25 bp, Artificial Sequence
  Synthetic Construct
aaataggcttgccactttctcttat

SEQ ID NO: 39 - DNA, 25 bp, Artificial Sequence
  Synthetic Construct
agttcagcacttgtctcattttaat

SEQ ID NO: 40 - DNA, 25 bp, Artificial Sequence
  Synthetic Construct
gtgacggacttctgaggctgtttcc

SEQ ID NO: 41 - DNA, 25 bp, Artificial Sequence
  Synthetic Construct
agtactcttaggtgccagtctggta

SEQ ID NO: 42 - DNA, 25 bp, Artificial Sequence
  Synthetic Construct
ccttccagggctgaccagtttgagt

SEQ ID NO: 43 - DNA, 25 bp, Artificial Sequence
  Synthetic Construct
ttcaggtactgtgtggtgaccgccc

SEQ ID NO: 44 - DNA, 25 bp, Artificial Sequence
  Synthetic Construct
gcaactacaatgactgtactctcta

SEQ ID NO: 45 - DNA, 25 bp, Artificial Sequence
  Synthetic Construct
gtcggacctgttccaggaggacctg

SEQ ID NO: 46 - DNA, 25 bp, Artificial Sequence
  Synthetic Construct
tgaactgactaattggtatccacta

SEQ ID NO: 47 - DNA, 25 bp, Artificial Sequence
  Synthetic Construct
tcagccagaggactttggaactgtg

SEQ ID NO: 48 - DNA, 25 bp, Artificial Sequence
  Synthetic Construct
agaaaaagcggtacgatgccttcct

SEQ ID NO: 49 - DNA, 25 bp, Artificial Sequence
  Synthetic Construct
ggtattgcacagttgtcactttatc

SEQ ID NO: 50 - DNA, 25 bp, Artificial Sequence
  Synthetic Construct
aatgatcaccatgttagccttagac

SEQ ID NO: 51 - DNA, 25 bp, Artificial Sequence
  Synthetic Construct
attttcttcttataaccatggcatc

SEQ ID NO: 52 - DNA, 25 bp, Artificial Sequence
  Synthetic Construct
ctgctgtgaccacaggctcaggtcc

SEQ ID NO: 53 - DNA, 25 bp, Artificial Sequence
  Synthetic Construct
agctacatactcctggcattgcact

SEQ ID NO: 54 - DNA, 25 bp, Artificial Sequence
  Synthetic Construct
gaacttaaatatatccccttcctca

SEQ ID NO: 55 - DNA, 25 bp, Artificial Sequence
  Synthetic Construct
gaagggagtgttgctcccagtccag

SEQ ID NO: 56 - DNA, 25 bp, Artificial Sequence
  Synthetic Construct
ccccgcaacagcttataccatggaa

SEQ ID NO: 57 - DNA, 25 bp, Artificial Sequence
  Synthetic Construct
tctttaggcctttcacaactaggac

SEQ ID NO: 58 - DNA, 25 bp, Artificial Sequence
  Synthetic Construct
gttgggtatcctactactttgtgtt

SEQ ID NO: 59 - DNA, 25 bp, Artificial Sequence
  Synthetic Construct
aggtgctctggactacatgtccttc

SEQ ID NO: 60 - DNA, 25 bp, Artificial Sequence
  Synthetic Construct
ataaagtacctttgagcatgagtgt

SEQ ID NO: 61 - DNA, 25 bp, Artificial Sequence
  Synthetic Construct
gactagtctctttatcagcacacac

SEQ ID NO: 62 - DNA, 25 bp, Artificial Sequence
  Synthetic Construct
cattagtattcctcattctgcatcc

SEQ ID NO: 63 - DNA, 25 bp, Artificial Sequence
  Synthetic Construct
acaccaaccaacattatttttgcaa

SEQ ID NO: 64 - DNA, 25 bp, Artificial Sequence
  Synthetic Construct
gactactcagaccagaacctgcagg

SEQ ID NO: 65 - DNA, 25 bp, Artificial Sequence
  Synthetic Construct
aagcatgacattcgttcacaatagc

SEQ ID NO: 66 - DNA, 25 bp, Artificial Sequence
  Synthetic Construct
cttttctccctggtggcgaggaagc

SEQ ID NO: 67 - DNA, 25 bp, Artificial Sequence
  Synthetic Construct
ctagatctatccaccttgttttttt

SEQ ID NO: 68 - DNA, 25 bp, Artificial Sequence
  Synthetic Construct
aacctactagtcttgaacaaactgt

SEQ ID NO: 69 - DNA, 25 bp, Artificial Sequence
  Synthetic Construct
ggacagcagactgccggtaacgcgc

SEQ ID NO: 70 - DNA, 25 bp, Artificial Sequence
  Synthetic Construct
gacacctcctacagcaagcaggtta

SEQ ID NO: 71 - DNA, 25 bp, Artificial Sequence
  Synthetic Construct
cagagagggacgacatcctgtgccc

SEQ ID NO: 72 - DNA, 25 bp, Artificial Sequence
  Synthetic Construct
tgcagttcctgagtaccttctacag

SEQ ID NO: 73 - DNA, 25 bp, Artificial Sequence
  Synthetic Construct
gatctgtgtcttgaagcagctgccc

SEQ ID NO: 74 - DNA, 25 bp, Artificial Sequence
  Synthetic Construct
ttgttctgaactttcctgttacctt

SEQ ID NO: 75 - DNA, 25 bp, Artificial Sequence
  Synthetic Construct
tgtgcctttattttatgagccccag

SEQ ID NO: 76 - DNA, 25 bp, Artificial Sequence
  Synthetic Construct
ggacctcaaccaggacttcagtgga

SEQ ID NO: 77 - DNA, 25 bp, Artificial Sequence
  Synthetic Construct
ttttgatctttccctatcttgatta

SEQ ID NO: 78 - DNA, 25 bp, Artificial Sequence
  Synthetic Construct
ccatcgcggtccacagtcaggatgt

SEQ ID NO: 79 - DNA, 25 bp, Artificial Sequence
  Synthetic Construct
tgaaacttaacccagctgtgttccc

SEQ ID NO: 80 - DNA, 25 bp, Artificial Sequence
  Synthetic Construct
aatagtgagaagctcgccctgtgtt

SEQ ID NO: 81 - DNA, 25 bp, Artificial Sequence
  Synthetic Construct
gttccccacaggatttcaacgtggc

SEQ ID NO: 82 - DNA, 25 bp, Artificial Sequence
  Synthetic Construct
ttgcagtgattccacggttaggccc

SEQ ID NO: 83 - DNA, 25 bp, Artificial Sequence
  Synthetic Construct
aagcagcgtttctatgacacatgca

SEQ ID NO: 84 - DNA, 25 bp, Artificial Sequence
  Synthetic Construct
tcagtgatccagggccgttcatgaa

SEQ ID NO: 85 - DNA, 25 bp, Artificial Sequence
  Synthetic Construct
ctggcctgttatgattctgaacatt

SEQ ID NO: 86 - DNA, 25 bp, Artificial Sequence
  Synthetic Construct
gtgttcagttctgttacacagtgca

SEQ ID NO: 87 - DNA, 25 bp, Artificial Sequence
  Synthetic Construct
gaagtatacacttccgctgtaccac

SEQ ID NO: 88 - DNA, 25 bp, Artificial Sequence
  Synthetic Construct
atccctgtttttaccatcaatcatc

SEQ ID NO: 89 - DNA, 25 bp, Artificial Sequence
  Synthetic Construct
gaggagctgagttggcagaccgggc

SEQ ID NO: 90 - DNA, 25 bp, Artificial Sequence
  Synthetic Construct
gctgcctgaaacgttgctttgtatt

SEQ ID NO: 91 - DNA, 25 bp, Artificial Sequence
  Synthetic Construct
cgcagacggtctgtgtgctgtttga

SEQ ID NO: 92 - DNA, 25 bp, Artificial Sequence
  Synthetic Construct
ggtcagtctaacagtttgcctgctg

SEQ ID NO: 93 - DNA, 25 bp, Artificial Sequence
  Synthetic Construct
cattagctcttagacccacagactg

SEQ ID NO: 94 - DNA, 25 bp, Artificial Sequence
  Synthetic Construct
gaaaaattgccttacgtacattcct

SEQ ID NO: 95 - DNA, 25 bp, Artificial Sequence
  Synthetic Construct
ggcctctggtctacaaagatttcat

SEQ ID NO: 96 - DNA, 25 bp, Artificial Sequence
  Synthetic Construct
tgaaactaacattccaagggtcagg

SEQ ID NO: 97 - DNA, 25 bp, Artificial Sequence
  Synthetic Construct
agtacccaaccatggccaaggtggc

SEQ ID NO: 98 - DNA, 25 bp, Artificial Sequence
  Synthetic Construct
tgcaagaatgcactttagaactatt

SEQ ID NO: 99 - DNA, 25 bp, Artificial Sequence
  Synthetic Construct
cgcagacggtctgtgtgctgtttga

SEQ ID NO: 100 - DNA, 25 bp, Artificial Sequence
  Synthetic Construct
cagcgagagcatgtccatctgttgg

SEQ ID NO: 101 - DNA, 25 bp, Artificial Sequence
  Synthetic Construct
tggactatgcacacttttaattttg

SEQ ID NO: 102 - DNA, 25 bp, Artificial Sequence
  Synthetic Construct
cttgtcctcagggctacagcagtag

SEQ ID NO: 103 - DNA, 25 bp, Artificial Sequence
  Synthetic Construct
acttgagctcagacctctaaaccct

SEQ ID NO: 104 - DNA, 25 bp, Artificial Sequence
  Synthetic Construct
atggcgggatcctccacagagagga

SEQ ID NO: 105 - DNA, 25 bp, Artificial Sequence
  Synthetic Construct
caacacgcaagttccttcttgaacc

SEQ ID NO: 106 - DNA, 25 bp, Artificial Sequence
  Synthetic Construct
ggcaccgaagtcagatgagtatccc

SEQ ID NO: 107 - DNA, 25 bp, Artificial Sequence
  Synthetic Construct
gagctggcgctcaaggatgctcgtg

SEQ ID NO: 108 - DNA, 25 bp, Artificial Sequence
  Synthetic Construct
aacagcaacttgtgttcctaacagg

SEQ ID NO: 109 - DNA, 25 bp, Artificial Sequence
  Synthetic Construct
ggagctctatccatattttactgat

SEQ ID NO: 110 - DNA, 25 bp, Artificial Sequence
  Synthetic Construct
agctgtcaccatgttatattttctt

SEQ ID NO: 111 - DNA, 25 bp, Artificial Sequence
  Synthetic Construct
ggaagtagagcatctctgtctcttt

SEQ ID NO: 112 - DNA, 25 bp, Artificial Sequence
  Synthetic Construct
gaggctatgatgttgatttcccacg

SEQ ID NO: 113 - DNA, 25 bp, Artificial Sequence
  Synthetic Construct
tctgcaccttatagactgatgtctc

SEQ ID NO: 114 - DNA, 25 bp, Artificial Sequence
  Synthetic Construct
gcatgtcaacagcgttcctagagaa

SEQ ID NO: 115 - DNA, 25 bp, Artificial Sequence
  Synthetic Construct
gcagaactgaaccggctttaccaaa

SEQ ID NO: 116 - DNA, 25 bp, Artificial Sequence
  Synthetic Construct
cagtggaaccaaatttttgccatta

SEQ ID NO: 117 - DNA, 25 bp, Artificial Sequence
  Synthetic Construct
tttagccctcatgactgtattttct

SEQ ID NO: 118 - DNA, 25 bp, Artificial Sequence
  Synthetic Construct
ccaccgaaggcctgatggctactca

SEQ ID NO: 119 - DNA, 25 bp, Artificial Sequence
  Synthetic Construct
atgatgcctctgtcaaggagtggac

SEQ ID NO: 120 - DNA, 25 bp, Artificial Sequence
  Synthetic Construct
tgacctcagtcaggcactggctgaa

SEQ ID NO: 121 - DNA, 25 bp, Artificial Sequence
  Synthetic Construct
ctggtcttaaagagtccctcacttc

SEQ ID NO: 122 - DNA, 25 bp, Artificial Sequence
  Synthetic Construct
gtatgtttctgtgtcaatcgctgtg

SEQ ID NO: 123 - DNA, 25 bp, Artificial Sequence
  Synthetic Construct
tccggagttatgcgtcctcttgaaa

SEQ ID NO: 124 - DNA, 25 bp, Artificial Sequence
  Synthetic Construct
acatggggtgacatgcctcgtatgt

SEQ ID NO: 125 - DNA, 25 bp, Artificial Sequence
  Synthetic Construct
cgcagtgggaacatcgagcacccgg

SEQ ID NO: 126 - DNA, 25 bp, Artificial Sequence
  Synthetic Construct
gaggatgccccaacaaactcatggc

SEQ ID NO: 127 - DNA, 25 bp, Artificial Sequence
  Synthetic Construct
aagtctacaggggctggtgacctct

SEQ ID NO: 128 - DNA, 25 bp, Artificial Sequence
  Synthetic Construct
gcagccaggatatgaccaccttact

SEQ ID NO: 129 - DNA, 25 bp, Artificial Sequence
  Synthetic Construct
atcctgcgcttgatcaactgaacca

SEQ ID NO: 130 - DNA, 25 bp, Artificial Sequence
  Synthetic Construct
gttagaatcgctgttctggcatcca

SEQ ID NO: 131 - DNA, 25 bp, Artificial Sequence
  Synthetic Construct
tattttcttattctggtataccaca

SEQ ID NO: 132 - DNA, 25 bp, Artificial Sequence
  Synthetic Construct
gcagcctccgggcgtgtgaagaaca

SEQ ID NO: 133 - DNA, 25 bp, Artificial Sequence
  Synthetic Construct
ctctttctagattgccagctcatga

SEQ ID NO: 134 - DNA, 25 bp, Artificial Sequence
  Synthetic Construct
tactaattccacaccttttattgac

SEQ ID NO: 135 - DNA, 25 bp, Artificial Sequence
  Synthetic Construct
gcctcgacacacataaaccttttta

SEQ ID NO: 136 - DNA, 25 bp, Artificial Sequence
  Synthetic Construct
gtgccctattgcgagcgagactatg

SEQ ID NO: 137 - DNA, 25 bp, Artificial Sequence
  Synthetic Construct
aaacatagcctatttttgtgcttaa

SEQ ID NO: 138 - DNA, 25 bp, Artificial Sequence
  Synthetic Construct
gattccaacttgtccaaaagctttc

SEQ ID NO: 139 - DNA, 25 bp, Artificial Sequence
  Synthetic Construct
ggaccttctgcatacctgtttagaa

SEQ ID NO: 140 - DNA, 25 bp, Artificial Sequence
  Synthetic Construct
aaggacctgatgccaacacaagtag

SEQ ID NO: 141 - DNA, 25 bp, Artificial Sequence
  Synthetic Construct
gccttttatatgggtcttcactctg

SEQ ID NO: 142 - DNA, 25 bp, Artificial Sequence
  Synthetic Construct
gcttgcacgtcttcgggtgcatgta

SEQ ID NO: 143 - DNA, 25 bp, Artificial Sequence
  Synthetic Construct
tccctttgcctgtggtgtaaaagtg

SEQ ID NO: 144 - DNA, 25 bp, Artificial Sequence
  Synthetic Construct
gcctcgacacacataaaccttttta

SEQ ID NO: 145 - DNA, 25 bp, Artificial Sequence
  Synthetic Construct
tacaggttccgcgtatgtgcgtgtc

SEQ ID NO: 146 - DNA, 25 bp, Artificial Sequence
  Synthetic Construct
ggctccagtcatgctgtgggatgag

SEQ ID NO: 147 - DNA, 25 bp, Artificial Sequence
  Synthetic Construct
cgctcctccctgataagagttgtcc

SEQ ID NO: 148 - DNA, 25 bp, Artificial Sequence
  Synthetic Construct
ggccgaaggctggtcatgagcatca

SEQ ID NO: 149 - DNA, 25 bp, Artificial Sequence
  Synthetic Construct
ttccgtttggtagactccttccaat

SEQ ID NO: 150 - DNA, 25 bp, Artificial Sequence
  Synthetic Construct
gcgaggctgcaaatttcatcatgaa

SEQ ID NO: 151 - DNA, 25 bp, Artificial Sequence
  Synthetic Construct
aagaaggccaatctctcatttgacc

SEQ ID NO: 152 - DNA, 25 bp, Artificial Sequence
  Synthetic Construct
gatcctttctgtaggctaattcctc

SEQ ID NO: 153 - DNA, 25 bp, Artificial Sequence
  Synthetic Construct
gatgggacgcataatcattacctta

SEQ ID NO: 154 - DNA, 25 bp, Artificial Sequence
  Synthetic Construct
aaagctggcaccagagacccgatag

SEQ ID NO: 155 - DNA, 25 bp, Artificial Sequence
  Synthetic Construct
cccaccatcatcaaacactcagtta

SEQ ID NO: 156 - DNA, 25 bp, Artificial Sequence
  Synthetic Construct
aatagaccctactgtactgtgcttt

SEQ ID NO: 157 - DNA, 25 bp, Artificial Sequence
  Synthetic Construct
tattccagaatgctgtacatctatt

SEQ ID NO: 158 - DNA, 25 bp, Artificial Sequence
  Synthetic Construct
ggcggctgctctgtcatgagaatgt

SEQ ID NO: 159 - DNA, 25 bp, Artificial Sequence
  Synthetic Construct
ccaagtatgtctagtcaccttttaa

SEQ ID NO: 160 - DNA, 25 bp, Artificial Sequence
  Synthetic Construct
aggcagctgggattcaattccctgc

SEQ ID NO: 161 - DNA, 25 bp, Artificial Sequence
  Synthetic Construct
tttgctatatccccaggccaaatag

SEQ ID NO: 162 - DNA, 25 bp, Artificial Sequence
  Synthetic Construct
gtctgagggtcttcctttatgcttg

SEQ ID NO: 163 - DNA, 25 bp, Artificial Sequence
  Synthetic Construct
ctagagaggccgcaaggggaccgcc

SEQ ID NO: 164 - DNA, 25 bp, Artificial Sequence
  Synthetic Construct
catggcccctcaatttatttgtggt

SEQ ID NO: 165 - DNA, 25 bp, Artificial Sequence
  Synthetic Construct
gtaggggggctgttagaattgctgc

SEQ ID NO: 166 - DNA, 25 bp, Artificial Sequence
  Synthetic Construct
tccccaaacttgctgtcaattccga

SEQ ID NO: 167 - DNA, 25 bp, Artificial Sequence
  Synthetic Construct
taccactggcatcgtgatggactcc

SEQ ID NO: 168 - DNA, 25 bp, Artificial Sequence
  Synthetic Construct
gcggctatgcaggtggtctgagctc

SEQ ID NO: 169 - DNA, 25 bp, Artificial Sequence
  Synthetic Construct
gacacttgccacactgtgtcgtcgt

SEQ ID NO: 170 - DNA, 25 bp, Artificial Sequence
  Synthetic Construct
gttaatgtcttaatccagcttgcaa

SEQ ID NO: 171 - DNA, 25 bp, Artificial Sequence
  Synthetic Construct
cttgggccgaagggttgctctgccc

SEQ ID NO: 172 - DNA, 25 bp, Artificial Sequence
  Synthetic Construct
tcaccaagtctggcggctcagatcg

SEQ ID NO: 173 - DNA, 25 bp, Artificial Sequence
  Synthetic Construct
ttatcaagtgtcaagatcagcaagt

SEQ ID NO: 174 - DNA, 25 bp, Artificial Sequence
  Synthetic Construct
gattacactggctgcagtacatcag

SEQ ID NO: 175 - DNA, 25 bp, Artificial Sequence
  Synthetic Construct
gcagccaaggtctgtgttcagcact

SEQ ID NO: 176 - DNA, 25 bp, Artificial Sequence
  Synthetic Construct
gtgtgatttttatagtctcgtttta

SEQ ID NO: 177 - DNA, 25 bp, Artificial Sequence
  Synthetic Construct
taccctgtggggatgtttcatactg

SEQ ID NO: 178 - DNA, 25 bp, Artificial Sequence
  Synthetic Construct
gaactgtcctaaggcaggggcgagc

SEQ ID NO: 179 - DNA, 25 bp, Artificial Sequence
  Synthetic Construct
aaataggaacacgtgctctacctcc

SEQ ID NO: 180 - DNA, 25 bp, Artificial Sequence
  Synthetic Construct
gtcgcctttatggaccacatacgga

SEQ ID NO: 181 - DNA, 25 bp, Artificial Sequence
  Synthetic Construct
agttctgaatgctgtcctcaaagta

SEQ ID NO: 182 - DNA, 25 bp, Artificial Sequence
  Synthetic Construct
gaatcagatggacactcacatggga

SEQ ID NO: 183 - DNA, 25 bp, Artificial Sequence
  Synthetic Construct
tcgatgagcgccacctctttaaaaa

SEQ ID NO: 184 - DNA, 25 bp, Artificial Sequence
  Synthetic Construct
cctgttcgcctttgtcatcaagatg

SEQ ID NO: 185 - DNA, 25 bp, Artificial Sequence
  Synthetic Construct
caagccacaggccttatttcatatg

SEQ ID NO: 186 - DNA, 25 bp, Artificial Sequence
  Synthetic Construct
taagtgcaacatttctgtatactgt

SEQ ID NO: 187 - DNA, 25 bp, Artificial Sequence
  Synthetic Construct
aaagcacacaacgattatagtaact

SEQ ID NO: 188 - DNA, 25 bp, Artificial Sequence
  Synthetic Construct
aacatgccattgatgcaaacgcagc

SEQ ID NO: 189 - DNA, 25 bp, Artificial Sequence
  Synthetic Construct
actgtgtttgatgtcctttattgat

SEQ ID NO: 190 - DNA, 25 bp, Artificial Sequence
  Synthetic Construct
gggcacctcagattgttgttgttaa

SEQ ID NO: 191 - DNA, 25 bp, Artificial Sequence
  Synthetic Construct
atcttccaaatggcagccctatccc

SEQ ID NO: 192 - DNA, 25 bp, Artificial Sequence
  Synthetic Construct
gtgggccagaagagttccttcctgg

SEQ ID NO: 193 - DNA, 25 bp, Artificial Sequence
  Synthetic Construct
gctgacctctgcaaggctttcaata